(19) United States
(12) Patent Application Publication
(10) Pub. No.: US 2025/0099232 A1
(43) Pub. Date: Mar. 27, 2025

(54) PROSTHETIC VALVES FOR DEPLOYMENT

(71) Applicant: EDWARDS LIFESCIENCES CORPORATION, Irvine, CA (US)

(72) Inventors: Matthew Michael Becerra, Lake Forest, CA (US); Yevgeniy Davidovich Kaufman, Denver, CO (US); Matthew A. Peterson, Costa Mesa, CA (US); David Robert Landon, Huntington Beach, CA (US); Wei Gan, Irvine, CA (US); Eric Robert Dixon, Villa Park, CA (US)

(21) Appl. No.: 18/907,484

(22) Filed: Oct. 5, 2024

Related U.S. Application Data
(63) Continuation of application No. PCT/US2023/016780, filed on Mar. 29, 2023.
(60) Provisional application No. 63/328,638, filed on Apr. 7, 2022.

(51) Int. Cl. A61F 2/24; A61B 17/00

(52) U.S. Cl. CPC A61F 2/2409; A61F 2/2418; A61F 2/243; A61B 2017/00243

(57) ABSTRACT

Apparatuses, systems, and methods for prosthetic valves. The prosthetic valves disclosed herein may be utilized for improved deployment, sealing, and anchoring to an implantation site. An implantation site may comprise a native heart valve or another implantation site in examples. Examples of prosthetic valves may include a proximal tensioning body positioned at a proximal portion of the prosthetic valve and a distal tensioning body positioned at a distal portion of the prosthetic valve. A sealing skirt may have a proximal end portion coupled to the proximal tensioning body and a distal end portion coupled to the distal tensioning body. The sealing skirt may be tensioned by the proximal tensioning body and the distal tensioning body and deflectable to conform to a shape of the native valve.

### CROSS REFERENCE TO RELATED APPLICATIONS

This application is a continuation of International Application No. PCT/US2023/016780, filed Mar. 29, 2023, which designates the United States and was published in English by the International Bureau on Oct. 12, 2023, as WO2023/196150, which claims the benefit of U.S. Provisional Application No. 63/328,638, filed Apr. 7, 2022, the entire contents of each of which are hereby incorporated by reference.

### BACKGROUND

### Field

Certain features of the disclosure relate generally to implants, including prosthetic valves for deployment.

### Background

Human heart valves, which include the aortic, pulmonary, mitral, and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves' ability to control blood flow. Such impairments reduce the heart's blood-pumping efficiency and can be a debilitating and life threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures.

These replacement valves are typically intended to replace the function of a native valve by blocking blood flow in a first direction while allowing blood to flow freely in a second direction. However, a problem occurs when blood leaks around the outside of the replacement valve. For example, in the context of replacement heart valves, paravalvular leakage (PVL) has proven particularly challenging. An additional challenge relates to the ability of such prostheses to be secured relative to intralumenal tissue, e.g., tissue within any body lumen or cavity, in an atraumatic manner.

### SUMMARY

Examples of prosthetic valves disclosed herein may be directed to improvements in prosthetic valves. Such prosthetic valves may comprise replacement heart valves in examples. Examples may be utilized for improved anchoring and sealing of flow (e.g., paravalvular leakage) outside of a flow channel of the prosthetic valve. Various other improvements are disclosed.

Examples may be directed to a prosthetic valve configured to be deployed to a native valve. The prosthetic valve may comprise one or more prosthetic valve leaflets; a sealing body positioned radially outward of the one or more prosthetic valve leaflets and configured to seal against a portion of the native valve; one or more anchors configured to anchor the one or more prosthetic valve leaflets to the native valve; and one or more magnets configured to draw the sealing body and at least one of the one or more anchors together.

Examples may be directed to a method. The method may comprise deploying a prosthetic valve to a native valve. The prosthetic valve may include one or more prosthetic valve leaflets, a sealing body positioned radially outward of the one or more prosthetic valve leaflets and configured to seal against a portion of the native valve, one or more anchors configured to anchor the one or more prosthetic valve leaflets to the native valve, and one or more magnets configured to draw the sealing body and at least one of the one or more anchors together.

Examples may be directed to a prosthetic valve configured to be deployed to a native valve. The prosthetic valve may comprise one or more prosthetic valve leaflets; and one or more anchors configured to anchor the one or more prosthetic valve leaflets to the native valve, each of the one or more anchors including one or more grooves configured to increase a flexibility of the respective anchor.

Examples may be directed to a method. The method may comprise deploying a prosthetic valve to a native valve. The prosthetic valve may include one or more prosthetic valve leaflets, and one or more anchors configured to anchor the one or more prosthetic valve leaflets to the native valve, each of the one or more anchors including one or more grooves configured to increase a flexibility of the respective anchor.

Examples may be directed to a prosthetic valve configured to be deployed to a native valve. The prosthetic valve may comprise one or more prosthetic valve leaflets; and one or more anchors configured to anchor the one or more prosthetic valve leaflets to the native valve, each of the one or more anchors including a coil.

Examples may be directed to a method. The method may comprise deploying a prosthetic valve to a native valve. The prosthetic valve may include one or more prosthetic valve leaflets, and one or more anchors configured to anchor the one or more prosthetic valve leaflets to the native valve, each of the one or more anchors including a coil.

Examples may be directed to a prosthetic valve configured to be deployed to a native valve. The prosthetic valve may comprise a valve body surrounding a flow channel extending along a longitudinal dimension; one or more prosthetic valve leaflets extending radially inward from the valve body and positioned within the flow channel; and one or more anchors pivotally coupled to the valve body and configured to pivot about the longitudinal dimension to vary a radial distance of the one or more anchors from the valve body.

Examples may be directed to a method. The method may comprise deploying a prosthetic valve to a native valve. The prosthetic valve may include a valve body surrounding a flow channel extending along a longitudinal dimension, one or more prosthetic valve leaflets extending radially inward from the valve body and positioned within the flow channel, and one or more anchors pivotally coupled to the valve body and configured to pivot about the longitudinal dimension to vary a radial distance of the one or more anchors from the valve body.

Examples may be directed to a prosthetic valve configured to be deployed to a native valve. The prosthetic valve may comprise one or more prosthetic valve leaflets; a sealing body positioned radially outward of the one or more prosthetic valve leaflets and configured to seal against a portion of the native valve; one or more anchors configured to anchor the one or more prosthetic valve leaflets to the native valve; and a band configured to have an adjustable diameter to move the sealing body radially inward or outward.

Examples may be directed to a method. The method may comprise deploying a prosthetic valve to a native valve. The prosthetic valve may include one or more prosthetic valve leaflets, a sealing body positioned radially outward of the one or more prosthetic valve leaflets and configured to seal against a portion of the native valve, one or more anchors configured to anchor the one or more prosthetic valve leaflets to the native valve, and a band configured to have an adjustable diameter to move the sealing body radially inward or outward.

Examples may be directed to a prosthetic valve configured to be deployed to a native valve. The prosthetic valve may comprise a valve frame surrounding a flow channel; one or more prosthetic valve leaflets coupled to the valve frame and extending radially inward from the valve frame and positioned within the flow channel; and a sealing skirt positioned radially outward from the valve frame with a space between the sealing skirt and the valve frame, the sealing skirt being in tension and configured to deflect to conform to a shape of the native valve.

Examples may be directed to a method. The method may comprise deploying a prosthetic valve to a native valve. The prosthetic valve may include a valve frame surrounding a flow channel, one or more prosthetic valve leaflets coupled to the valve frame and extending radially inward from the valve frame and positioned within the flow channel, and a sealing skirt positioned radially outward from the valve frame with a space between the sealing skirt and the valve frame, the sealing skirt being in tension and configured to deflect to conform to a shape of the native valve.

Examples may be directed to a prosthetic valve configured to be deployed to a native valve. The prosthetic valve may comprise a valve frame surrounding a flow channel; one or more prosthetic valve leaflets coupled to the valve frame and extending radially inward from the valve frame and positioned within the flow channel; a sealing skirt positioned radially outward from the valve frame and including a pocket; and a ring positioned in the pocket and configured to support the sealing skirt.

Examples may be directed to a method. The method may comprise deploying a prosthetic valve to a native valve. The prosthetic valve may include a valve frame surrounding a flow channel, one or more prosthetic valve leaflets coupled to the valve frame and extending radially inward from the valve frame and positioned within the flow channel, a sealing skirt positioned radially outward from the valve frame and including a pocket, and a ring positioned in the pocket and configured to support the sealing skirt.

Examples may be directed to a prosthetic valve configured to be deployed to a native valve. The prosthetic valve may comprise a valve frame surrounding a flow channel; one or more prosthetic valve leaflets coupled to the valve frame and extending radially inward from the valve frame and positioned within the flow channel; one or more anchors configured to anchor the one or more prosthetic valve leaflets to the native valve; a sealing skirt positioned radially outward from the valve frame and including a distal portion; and one or more slidable couplers coupling the distal portion of the sealing skirt to the one or more anchors and configured to allow the distal portion of the sealing skirt to slide relative to the one or more anchors.

Examples may be directed to a method. The method may comprise deploying a prosthetic valve to a native valve. The prosthetic valve may include a valve frame surrounding a flow channel, one or more prosthetic valve leaflets coupled to the valve frame and extending radially inward from the valve frame and positioned within the flow channel, one or more anchors configured to anchor the one or more prosthetic valve leaflets to the native valve, a sealing skirt positioned radially outward from the valve frame and including a distal portion, and one or more slidable couplers coupling the distal portion of the sealing skirt to the one or more anchors and configured to allow the distal portion of the sealing skirt to slide relative to the one or more anchors.

Examples may be directed to a prosthetic valve configured to be deployed to a native valve. The prosthetic valve may comprise one or more prosthetic valve leaflets; and one or more anchors configured to anchor the one or more prosthetic valve leaflets to the native valve, each of the one or more anchors comprising a T-bar.

Examples may be directed to a method. The method may comprise deploying a prosthetic valve to a native valve. The prosthetic valve may include one or more prosthetic valve leaflets, and one or more anchors configured to anchor the one or more prosthetic valve leaflets to the native valve, each of the one or more anchors comprising a T-bar.

Examples may be directed to a prosthetic valve configured to be deployed to a native valve. The prosthetic valve may comprise one or more prosthetic valve leaflets; a valve body supporting the one or more prosthetic valve leaflets; a plurality of distal anchors each comprising an elongate arm having a first end coupled to the valve body and extending radially outward along a length from the first end to a tip of the respective elongate arm, each elongate arm configured to configured to hook over a distal tip of a native valve leaflet to anchor the one or more prosthetic valve leaflets to the native valve; and a sealing skirt forming a disk extending along the length of each of the elongate arms and circumferentially between adjacent of the elongate arms.

Examples may be directed to a method. The method may comprise deploying a prosthetic valve to a native valve. The prosthetic valve may include one or more prosthetic valve leaflets, a valve body supporting the one or more prosthetic valve leaflets, a plurality of distal anchors each comprising an elongate arm having a first end coupled to the valve body and extending radially outward along a length from the first end to a tip of the respective elongate arm, each elongate arm configured to configured to hook over a distal tip of a native valve leaflet to anchor the one or more prosthetic valve leaflets to the native valve, and a sealing skirt forming a disk extending along the length of each of the elongate arms and circumferentially between adjacent of the elongate arms.

Examples may be directed to a prosthetic valve configured to be deployed to a native valve. The prosthetic valve may comprise one or more prosthetic valve leaflets; and one or more anchors configured to anchor the one or more prosthetic valve leaflets to the native valve, each of the one or more anchors including a hinge configured to allow the respective anchor to deflect radially.

Examples may be directed to a method. The method may comprise deploying a prosthetic valve to a native valve, the prosthetic valve including: one or more prosthetic valve leaflets, and one or more anchors configured to anchor the one or more prosthetic valve leaflets to the native valve, each of the one or more anchors including a hinge configured to allow the respective anchor to deflect radially.

Examples may be directed to a prosthetic valve configured to be deployed to a native valve. The prosthetic valve may comprise one or more prosthetic valve leaflets; an inner frame supporting the one or more prosthetic valve leaflets; an outer frame positioned radially outward from the inner frame and surrounding the inner frame, the outer frame being separated from the inner frame with a gap; and one or more anchors coupled to the outer frame and each configured to be positioned radially outward of one or more leaflets of the native valve to anchor to the native valve.

Examples may be directed to a method. The method may comprise deploying a prosthetic valve to a native valve, the prosthetic valve including: one or more prosthetic valve leaflets, an inner frame supporting the one or more prosthetic valve leaflets, an outer frame positioned radially outward from the inner frame and surrounding the inner frame, the outer frame being separated from the inner frame with a gap, and one or more anchors coupled to the outer frame and each configured to be positioned radially outward of one or more leaflets of the native valve to anchor to the native valve.

Examples may be directed to a prosthetic valve configured to be deployed to a native valve. The prosthetic valve may comprise one or more prosthetic valve leaflets; and a frame including an outer frame and an inner frame, the outer frame and the inner frame being cut from a single piece of material such that the outer frame is integral with the inner frame, the outer frame being deflected radially outward from the inner frame and including a plurality of struts bounding a plurality of openings, the inner frame configured to support the one or more prosthetic valve leaflets and including a plurality of struts bounding a plurality of openings, and at least a portion of the inner frame is aligned within at least one of the plurality of openings of the outer frame, or at least a portion of the outer frame is aligned within at least one of the plurality of openings of the inner frame.

Examples may be directed to a method. The method may comprise deploying a prosthetic valve to a native valve, the prosthetic valve including: one or more prosthetic valve leaflets, a frame including an outer frame and an inner frame, the outer frame and the inner frame being cut from a single piece of material such that the outer frame is integral with the inner frame, the outer frame being deflected radially outward from the inner frame and including a plurality of struts bounding a plurality of openings, the inner frame configured to support the one or more prosthetic valve leaflets and including a plurality of struts bounding a plurality of openings, and at least a portion of the inner frame is aligned within at least one of the plurality of openings of the outer frame, or at least a portion of the outer frame is aligned within at least one of the plurality of openings of the inner frame.

Any of the features of an example disclosed herein, is applicable to all other aspects and examples identified herein. Moreover, any of the features of an example of the various examples, is independently combinable, partly or wholly with other examples described herein in any way, e.g., one, two, or three or more examples may be combinable in whole or in part. Further, any of the features of an example may be made optional to other aspects or examples. Any example of a method can be performed by a system or apparatus of another example, and any example of a system or apparatus can be configured to perform a method of another example.

### BRIEF DESCRIPTION OF THE DRAWINGS

Features and advantages of the systems, apparatuses, and methods as disclosed herein will become appreciated as the same become better understood with reference to the specification, claims, and appended drawings wherein:

FIG. 1 illustrates an upper perspective view of a prosthetic valve according to examples of the present disclosure.

FIG. 2 illustrates a bottom perspective view of the prosthetic valve shown in FIG. 1.

FIG. 3 illustrates a side cross sectional schematic view of the prosthetic valve shown in FIG. 1.

FIG. 4 illustrates a schematic view of a delivery apparatus approaching an implantation site.

FIG. 5 illustrates a side cross sectional schematic view of the prosthetic valve shown in FIG. 1 positioned within a delivery apparatus.

FIG. 6 illustrates a side cross sectional schematic view of the prosthetic valve shown in FIG. 1 being deployed to a native heart valve.

FIG. 7 illustrates a side cross sectional schematic view of the prosthetic valve shown in FIG. 1 deployed to a native heart valve.

FIG. 8 illustrates a detail view of magnetic attraction between an anchor and a sealing body shown in FIG. 7.

FIG. 9 illustrates a side cross sectional view of an anchor.

FIG. 10 illustrates a side cross sectional view of an anchor.

FIG. 11 illustrates a side view of an anchor.

FIG. 12 illustrates a side cross sectional view of the anchor shown in FIG. 11.

FIG. 13 illustrates a cross sectional view along line A-A shown in FIG. 12.

FIG. 14 illustrates a perspective view of a prosthetic valve.

FIG. 15 illustrates a top cross sectional view of the prosthetic valve shown in FIG. 14.

FIG. 16 illustrates a top cross sectional view of the prosthetic valve shown in FIG. 14.

FIG. 17 illustrates a top cross sectional view of the prosthetic valve shown in FIG. 14, with native valve leaflets shown in dashed lines.

FIG. 18 illustrates a top cross sectional view of a prosthetic valve.

FIG. 19 illustrates a side cross sectional view of a lock.

FIG. 20 illustrates a side cross sectional view of the lock shown in FIG. 19.

FIG. 21 illustrates a perspective view of a prosthetic valve.

FIG. 22 illustrates a partial cross sectional perspective view of a prosthetic valve.

FIG. 23 illustrates a partial cross sectional perspective view of the prosthetic valve shown in FIG. 22.

FIG. 24 illustrates a top cross sectional view of the prosthetic valve shown in FIG. 22.

FIG. 25 illustrates a top view of a band.

FIG. 26 illustrates a schematic view of a portion of a band.

FIG. 27 illustrates a side cross sectional view of a prosthetic valve.

FIG. 28 illustrates a side cross sectional view of the prosthetic valve shown in FIG. 27 moved from the position shown in FIG. 27.

FIG. 29 illustrates a perspective view of the prosthetic valve shown in FIG. 27.

FIG. 30 illustrates a side cross sectional view of the prosthetic valve shown in FIG. 27 deployed to a native heart valve.

FIG. 31 illustrates a perspective view of a prosthetic valve.

FIG. 32 illustrates a side cross sectional view of the prosthetic valve shown in FIG. 31.

FIG. 33 illustrates a perspective view of a prosthetic valve.

FIG. 34 illustrates a perspective view of a prosthetic valve.

FIG. 35 illustrates a perspective view of a prosthetic valve.

FIG. 36 illustrates a side cross sectional view of a prosthetic valve.

FIG. 37 illustrates a top view of a ring.

FIG. 38 illustrates a side cross sectional view of a prosthetic valve.

FIG. 39 illustrates a side cross sectional view of a prosthetic valve.

FIG. 40 illustrates a side cross sectional view of a slidable coupler sliding along an anchor.

FIG. 41 illustrates a side cross sectional view of a slidable coupler sliding along an anchor.

FIG. 42 illustrates a side cross sectional view of a prosthetic valve.

FIG. 43 illustrates a top schematic view of the prosthetic valve shown in FIG. 42.

FIG. 44 illustrates a side view of the prosthetic valve shown in FIG. 42 deployed to an implantation site.

FIG. 45 illustrates a perspective view of a prosthetic valve.

FIG. 46 illustrates a perspective view of an anchor of the prosthetic valve shown in FIG. 45.

FIG. 47 illustrates a top schematic view of the prosthetic valve shown in FIG. 45.

FIG. 48 illustrates a top view of an anchor shown in FIG. 45.

FIG. 49 illustrates a top view of an anchor shown in FIG. 45.

FIG. 50 illustrates a top view of an anchor.

FIG. 51 illustrates a top schematic view of a portion of a prosthetic valve.

FIG. 52 illustrates a perspective view of the portion of the prosthetic valve shown in FIG. 51.

FIG. 53 illustrates a cross sectional perspective view of a prosthetic valve.

FIG. 54 illustrates a side cross sectional view of the prosthetic valve shown in FIG. 53 deployed to an implantation site.

FIGS. 55A and 55B each illustrate a side cross sectional view of an anchor.

FIG. 56 illustrates a side cross sectional view of an anchor.

FIG. 57 illustrates a side cross sectional schematic view of a prosthetic valve positioned within a delivery apparatus.

FIG. 58 illustrates a side cross sectional schematic view of the prosthetic valve shown in FIG. 57 being deployed to a native heart valve.

FIG. 59 illustrates a side cross sectional schematic view of the prosthetic valve shown in FIG. 57 being deployed to a native heart valve.

FIG. 60 illustrates a side cross sectional schematic view of the prosthetic valve shown in FIG. 57 being deployed to a native heart valve.

FIG. 61 illustrates a side cross sectional schematic view of the prosthetic valve shown in FIG. 57 being deployed to a native heart valve.

FIG. 62 illustrates a side cross sectional schematic view of the prosthetic valve shown in FIG. 57 deployed to a native heart valve.

FIG. 63 illustrates a side view of an inner frame.

FIG. 64 illustrates a side view of an outer frame.

FIG. 65 illustrates a perspective view of the outer frame shown in FIG. 64 coupled to the inner frame shown in FIG. 63.

FIG. 66 illustrates a side view of the outer frame shown in FIG. 64 coupled to the inner frame shown in FIG. 63.

FIG. 67 illustrates a top view of the outer frame shown in FIG. 64 coupled to the inner frame shown in FIG. 63.

FIG. 68 illustrates a side cross sectional schematic view of a prosthetic valve deployed to a native heart valve.

FIG. 69 illustrates a side cross sectional view of an anchor and a portion of a prosthetic valve.

FIG. 70 illustrates a side cross sectional view of the anchor shown in FIG. 69 anchored to a native valve leaflet.

FIG. 71 illustrates a perspective view of an outer frame.

FIG. 72 illustrates a side cross sectional schematic view of a portion of the outer frame shown in FIG. 71.

FIG. 73 illustrates a side cross sectional schematic view of a prosthetic valve positioned within a delivery apparatus.

FIG. 74 illustrates a side cross sectional schematic view of a prosthetic valve being deployed from a delivery apparatus.

FIG. 75 illustrates a side cross sectional schematic view of a prosthetic valve being deployed from a delivery apparatus.

FIG. 76 illustrates a side cross sectional schematic view of the prosthetic valve shown in FIG. 73 deployed to a native heart valve.

FIG. 77 illustrates a perspective view of a frame.

FIG. 78 illustrates a perspective view of the frame shown in FIG. 77 rotated from the position shown in FIG. 77.

FIG. 79 illustrates a side view of the frame shown in FIG. 77.

FIG. 80 illustrates a side view of the frame shown in FIG. 77 rotated from the position shown in FIG. 77.

FIG. 81 illustrates a top view of the frame shown in FIG. 77.

FIG. 82 illustrates a side view of the inner frame shown in FIG. 77 isolated from the outer frame shown in FIG. 77.

FIG. 83 illustrates a side view of the outer frame shown in FIG. 77 isolated from the inner frame shown in FIG. 77.

FIG. 84 illustrates a side view of the frame shown in FIG. 77 in a crimped or compressed configuration.

FIG. 85 illustrates a side view of the frame shown in FIG. 77 in a crimped or compressed configuration and rotated from the position shown in FIG. 84.

FIG. 86 illustrates a top schematic view of the frame shown in FIG. 77 in a crimped or compressed configuration.

FIG. 87 illustrates a perspective view of a frame.

FIG. 88 illustrates a perspective view of a frame.

FIG. 89 illustrates a detail view of a portion of a frame shown in FIG. 88.

FIG. 90 illustrates a side cross sectional schematic view of a prosthetic valve.

FIG. 91 illustrates a side view of strut cells of a prosthetic valve.

FIG. 92 illustrates a side schematic view of a portion of a prosthetic valve.

FIG. 93 illustrates a side cross sectional schematic view of a prosthetic valve.

### DETAILED DESCRIPTION

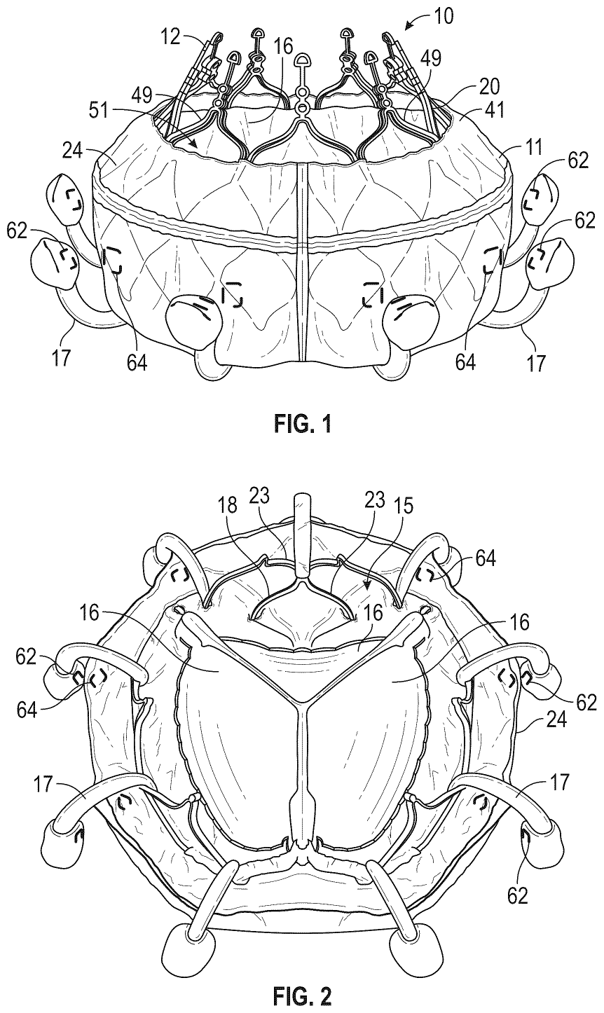

FIG. 1 illustrates a perspective view of a prosthetic valve 10 in the form of a replacement heart valve. The prosthetic valve 10 may be configured to be deployed within a portion of a patient's body. The prosthetic valve 10, for example, may be deployed within an annulus of a native valve, such as a native mitral valve or a native tricuspid valve. In examples, other implantation locations may be utilized such as within an aortic or pulmonary valve, or in other valves or locations within a patient's body as desired.

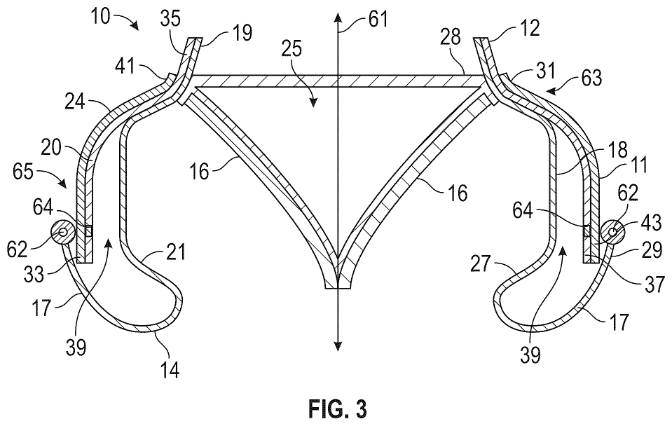

The prosthetic valve 10 may include a proximal end 12 and a distal end 14 (marked in FIG. 3), and a length therebetween. The prosthetic valve 10 may further include one or more prosthetic valve leaflets 16, or a plurality of prosthetic valve leaflets 16, configured to surround a flow channel for controlling flow through the valve 10. The prosthetic valve leaflets 16 are configured to move between opened and closed states to mimic and replace the operation of native valve leaflets. In examples, the prosthetic valve leaflets 16 may be coupled to a frame or to an intermediate fabric portion, which connects the leaflets to the frame. The frame may include an inner frame 18, as shown in FIG. 2 and in the cross sectional view of FIG. 3, and may include an outer frame 20 as shown in FIGS. 1 and 3, which may be part of a sealing body 11 and may be spaced radially outwardly from the inner frame 18.

FIG. 3 illustrates a cross sectional schematic view of the prosthetic valve 10. The inner frame 18 includes a proximal portion having a proximal end 19 and a distal portion having a distal end 21. The inner frame 18 may have a bulbous shape, comprising a curved body that curves radially outwardly between the proximal end 19 and the distal end 21, or may have another configuration in examples as desired. The inner frame 18 may have a circular shape in examples. The prosthetic valve leaflets 16 are positioned within the inner frame 18.

Referring to FIG. 2, the inner frame 18 may include a plurality of struts 23 with spaces 15 therebetween. Such a configuration may allow the inner frame 18 to move between an undeployed, unexpanded, or linearized configuration to a deployed or expanded configuration. For example, the inner frame 18 may expand radially outward to move to the deployed or expanded configuration, with the length of the inner frame 18 decreasing due to the increased diameter of the inner frame 18. Other configurations of inner frames 18 may be utilized as desired.

Referring to FIGS. 1 and 3, the prosthetic valve 10 preferably includes one or more anchors 17 that may be coupled to the prosthetic valve leaflets 16. The anchors 17 are shaped to anchor the prosthetic valve 10 to a portion of a patient's heart, such as a native valve. The anchors 17 may particularly be configured to anchor to the native valve leaflets of the patient's heart. The anchors 17 may extend around the native valve leaflets to anchor to the native valve leaflets. The anchors 17 may comprise distal anchors positioned at the distal end 14 of the valve 10, or in examples may be positioned in another position, such as along an intermediate region of the valve 10.

Each anchor 17 may be configured as a protruding arm configured to extend distally and then curve in a proximal direction to the tip of the respective one of the anchors 17. Such a configuration may allow the anchor 17 to extend around a native leaflet and around the distal tip of the leaflet, to hook over the distal tip of the native valve leaflet and be positioned radially outward of an outward facing surface of a leaflet of the native valve. The anchors 17 may be configured to be in a hooked configuration as shown in FIGS. 1-3 for example. The anchor 17 may thus resist a force applied in the atrial or proximal direction to the valve 10 and may anchor the valve 10 within the native valve annulus. Other configurations of anchors 17 may be utilized in examples as desired.

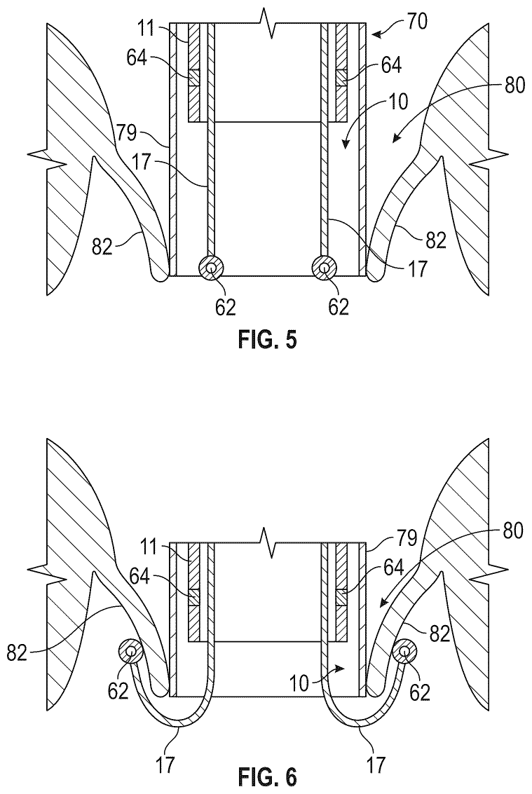

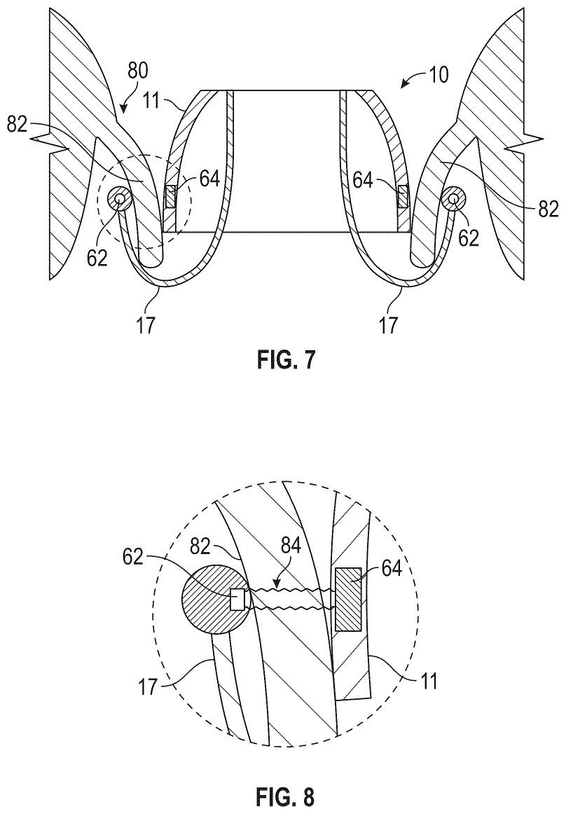

The anchors 17 are shown in FIGS. 1-3 in a deployed or expanded configuration, in which the tips of the anchors 17 extend proximally. In examples, the anchors 17 may be configured to be in undeployed, unexpanded, or linearized configuration in which the tips of the anchors 17 extend distally. Such a configuration is shown in FIG. 5 for example. The anchors 17 may be configured to be flexible in examples. Upon deployment, the anchors 17 may be configured to move from the undeployed configuration radially outward to the deployed configuration, with the tips flipped towards the proximal direction. Such an operation may allow the anchors 17 to flip behind the native valve leaflets for capturing the native valve leaflets during deployment. Such a configuration is shown in FIG. 7, wherein the native leaflets 82 are located between the anchors 17 and the outer sealing frame 11. Other deployment methods for the anchors 17 may be utilized in examples as desired.

Referring to FIG. 3, the prosthetic valve leaflets 16 are supported within the inner frame 18. For example, the proximal portion of the inner frame 18 may be coupled to a proximal portion of the plurality of prosthetic valve leaflets 16. The prosthetic valve leaflets 16 may be coupled to the inner frame 18 and may extend radially inward from the inner frame 18. The prosthetic valve leaflets 16 may couple to the valve frame 18 via an intermediate body 28 that may support the prosthetic valve leaflets 16 and may couple the leaflets 16 to the inner frame 18 via sutures or another method as desired.

The prosthetic valve leaflets 16 surround a flow channel 25 as marked in FIG. 3 and may move between open and closed states to control flow through the flow channel 25. As shown in FIG. 3, the proximal end of the prosthetic valve 10 may comprise an inflow end of the valve 10, and the distal end of the prosthetic valve 10 may comprise an outflow end, although other configurations may be utilized as desired. The prosthetic valve leaflets 16 may be positioned around a central axis 61 of the prosthetic valve 10. The inner frame 18 and outer frame 20 may each surround the central axis 61 of the prosthetic valve 10.

The anchors 17 may each extend radially outwardly from the flow channel 25 and radially outwardly from the prosthetic valve leaflets 16 of the valve 10. The anchors 17 may be coupled to the distal portion of the inner frame 18. The anchors 17 may each include a proximal portion 27 and a distal portion 29, with the proximal portion 27 coupled to the inner frame 18 and the distal portion 29 comprising a tip of the respective anchor 17. The anchors 17 may extend vertically from the proximal portion 27 to the tip at the distal portion 29 when the valve 10 is deployed.

Referring again to FIG. 1, the prosthetic valve 10 preferably includes an outer sealing body 11. The sealing body 11 may be positioned radially outwardly from the prosthetic valve leaflets 16 and may be configured to seal against a portion of the native valve. The sealing body 11 forms the outer surface of the valve 10. The sealing body 11 may define the outer diameter of the valve 10 and may comprise the outer periphery of the valve 10. The sealing body 11 includes a proximal portion having a proximal end 31 and a distal portion having a distal end 33 (marked in FIG. 3).

Referring to the cross sectional view of FIG. 3, the sealing body 11 includes a frame 20 and a sealing skirt 24, or in examples may comprise only a frame or only a sealing skirt as desired. In the illustrated embodiment, the frame 20 comprises an outer frame that is positioned radially outwardly from the inner frame 18. The sealing skirt 24 is coupled to the outer frame 20 and forms the outer portion of the sealing body 11, as shown in FIG. 1.

At least a portion of the sealing body 11 is configured to engage surrounding tissue and thereby form a seal. The outer frame 20 is positioned radially outwardly of the inner frame 18. The outer frame 20 may have a proximal portion 35 that couples to the proximal end 19 of the inner frame 18. The proximal portion 35 may extend radially outward from the proximal end 19 of the inner frame 18 and from the prosthetic valve leaflets 16. A distal portion 37 of the outer frame 20 may be spaced from the prosthetic valve leaflets 16 and the inner frame 18 at a gap 39. The gap 39 may be positioned between the outer frame 20 of the sealing body 11 and a distal portion of the inner frame 18. The inner frame 18 accordingly may comprise an inner frame and the frame 20 of the sealing body 11 may comprise an outer frame positioned radially outward of the inner valve frame 18 and surrounding the inner frame 18 and the prosthetic valve leaflets 16.

The outer frame 20 may have a length that extends distally to a lesser distance than the distal end of the inner frame 18. As such, the outer frame 20 may be shorter than the inner frame 18 in the deployed state. The outer frame 20 may further have a curved configuration that curves outward from the inner frame 18, where a largest diameter of the outer frame 20 is located along the distal portion of the outer frame 20.

The outer frame 20 of the sealing body 11 includes a plurality of struts 49 (as marked in FIG. 1) forming the frame 20, with spaces 51 between the struts. Such a configuration utilized with the frame 20 may allow the frame 20 to move between an undeployed, unexpanded, or linearized configuration to a deployed or expanded configuration as shown in FIG. 1, in which the outer frame 20 and sealing body 11 have a curved bulbous shape. As with the valve frame 18, the length of the outer frame 20 of the sealing body 11 may decrease as the diameter of the outer frame 20 of the sealing body 11 increases during deployment. The diameter of the outer frame 20 of the sealing body 11 may radially expand outward from the inner valve frame 18 simultaneously, or at a different time or rate of expansion as the inner valve frame 18 in examples.

The sealing body 11 may include a sealing skirt 24 (as shown in FIG. 1) that may extend around the inner valve frame 18 and the prosthetic valve leaflets 16. The skirt 24 may be directly coupled to the frame 20 of the sealing body or may be free to expand outwardly from the frame 20.

The sealing skirt 24 has a proximal portion 41 (marked in FIG. 3) that is coupled to the proximal portion of the sealing body 11 and may also be coupled to the proximal portion of the inner frame 18. The skirt 24 may have a distal portion 43 (marked in FIG. 3) that may be coupled to the distal end of the frame 20, and in examples may be coupled to the inner valve frame 18 or one or more of the anchors 17. As shown in FIG. 3, the anchors 17 are configured to extend radially outwardly from the inner valve frame 18 and across the gap 39 to the tip of the respective anchor 17.

The sealing skirt 24 is preferably made from a material that resists fluid flow therethrough, such as a cloth material, woven material, or other material such as a polymer or other material that resists fluid flow therethrough. The material may comprise a fabric. A variety of materials may be utilized for the skirt 24 as desired.

The sealing body 11 may be configured to abut a portion of the patient's heart to reduce fluid flow. The skirt 24 may be configured to seal a portion of the native valve annulus. For example, the sealing body 11 may abut a surface of a patient's native valve leaflet to reduce fluid flow between the sealing body 11 and the native leaflet. The sealing body 11 may be configured to abut other portions of the patient's heart to reduce fluid flow as desired.

In examples, the sealing body 11 may be flexible to allow for movement and conformability to a native valve annulus.

In examples, one or more magnets may be utilized for drawing portions of the prosthetic valve 10 towards each other. For example, one or more magnets may be configured to draw the anchors 17 toward the sealing body 11. Magnets may also be provided in other positions for pulling other portions of the prosthetic valve together as desired.

Referring to FIG. 1, the one or more magnets may be positioned in a variety of locations on the prosthetic valve 10. In one example, one or more of the magnets are incorporated within or attached to the anchors 17. A magnet 62 may be positioned on each of the anchors 17 and is preferably positioned on the tip of the anchor 17. In examples, each magnet 62 may be positioned on an anchor 17 such that the magnets 62 are circumferentially spaced from each other and spaced radially outwardly from the sealing body 11.

In examples, one or more magnets 64 may be positioned on the sealing body 11. The magnets 64 may be positioned on the sealing body 11 and circumferentially spaced from each other. The magnets 64 may be positioned in circumferential alignment with the magnets 62 on the anchors 17 in examples, or may be in another position. For example, the magnets 64 may be adjacent to the magnets 62 yet on another side of a native valve leaflet. The magnets 64 may be positioned proximate the magnets 62 on the anchors 17. The magnets 64 may be coupled to the outer frame 20 or may be embedded in the sealing skirt 24 or otherwise may be coupled to the sealing body 11. The outer frame 20 may include a proximal portion 63 that may be coupled to the inner frame 18 and a distal portion 65. The magnets 64 may be positioned on the distal portion 65 or in another location in examples.

In examples, the magnets 62 on the anchors 17 are configured to interact with opposing magnets 64 located on the sealing body 11. The polarity of the magnets 62, 64, for example, may be set such that an attractive magnetic force is provided between the magnets 62, 64 to draw the magnets 62, 64 towards each other. In a corresponding manner, the respective anchors 17 and sealing body 11 may be drawn towards each other.

In examples, magnetic responsive materials such as ferromagnetic materials may be utilized in combination or in substitution with a magnet. For example, a ferromagnetic material such as iron, or another metal or an alloy, may be utilized to attract with one of the magnets 62, 64. A ferromagnetic material may be substituted for the magnet 62 on the anchor 17, for example, and may be attracted to the magnet 64 on the sealing body 11. Similarly, a ferromagnetic material may be substituted for the magnet 64 on the sealing body 11 and may be attracted to the magnet 62 on the anchor 17. Combinations of magnets and ferromagnetic materials may be utilized in examples as desired.

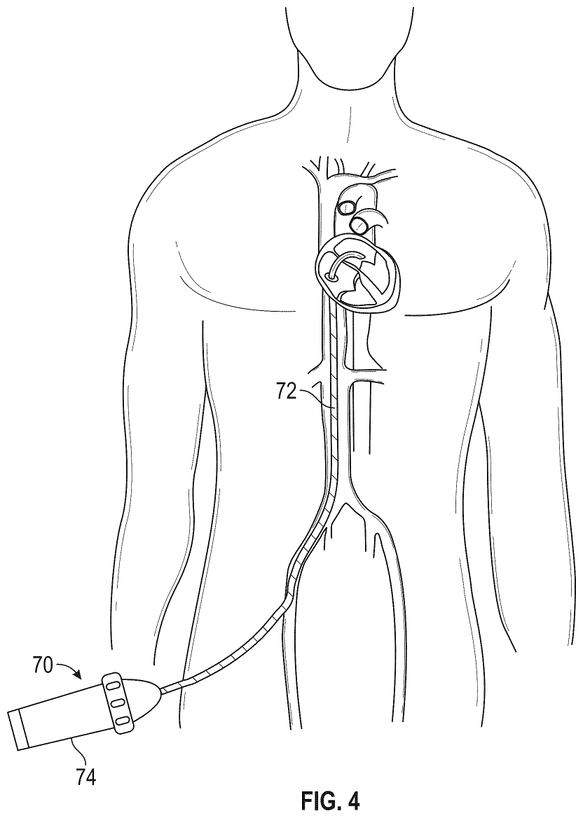

FIG. 4 illustrates advancement of a delivery system 70 for deployment of the prosthetic valve 10 to an implantation site. The delivery system 70 may include an elongate shaft 72 having a proximal portion and a distal portion, with the proximal portion coupled to a housing in the form of a handle 74. The delivery system 70 may be advanced through the vasculature of a patient, such as the femoral vein as shown in FIG. 4. Other entries may be utilized in examples, including transapical, or via surgical methods such as thoracotomy or open heart surgery.

The prosthetic valve 10 may be positioned within an implant retention area of the delivery system 70 and may be covered with a capsule or may otherwise be retained prior to deployment. The prosthetic valve 10 may be deployed as a self-expanding prosthetic or may be a balloon-expandable prosthetic (e.g., positioned upon an inflatable balloon upon entry into the patient's body, or slid onto an inflatable balloon within the patient's body), or may be mechanically expanded, among other forms of deployment.

The delivery system 70 may be advanced to pass into an atrium of a heart and may pass transeptally into another atrium (e.g., from the right atrium to the left atrium) to reach an implantation site. Such a delivery approach may be utilized for mitral native valve access for example. In examples, the delivery system 70 may extend to the right atrium for tricuspid access, or other delivery approaches to other implantation sites may be utilized in examples as desired.

FIG. 5 illustrates a side cross sectional view of the prosthetic valve 10 in a compressed configuration within a capsule 79 of the delivery system 70. The anchors 17 are shown to extend longitudinally in an elongated configuration. The prosthetic valve 10 may be positioned to be deployed to a native valve 80.

FIG. 6 illustrates the prosthetic valve 10 partially deployed to a native valve 80. The anchors 17 may be advanced and may deploy radially outward from the capsule 79. The anchors 17 may hook over native valve leaflets 82 such that the tips of the anchors 17 are positioned radially outward of the native valve leaflets 82. The magnets 62 coupled to the anchors 17 may accordingly be positioned radially outward of the native valve leaflets 82.

FIG. 7 illustrates the prosthetic valve 10 deployed to the native valve 80. The magnets 62 coupled to the anchors 17 are positioned radially outward of the native valve leaflets 82. The magnets 64 coupled to the sealing body 11 are positioned radially inward of the native valve leaflets 82. The magnets 62, 64 may be circumferentially aligned with each other and may be axially aligned with each other to provide an attractive magnetic force between the magnets 62, 64.

FIG. 8 provides an enlarged view of the magnets 62, 64 interacting with each other. The magnets 62, 64 may be positioned on opposite sides of the native valve leaflets 82 to provide an attractive magnetic force (represented with lines 84) through the native valve leaflet 82.

The magnets 62, 64 may be configured to enhance clamping of the native valve leaflet 82 between the sealing body 11 and the anchors 17. The additional clamping force applied by the magnets 62, 64 may provide a variety of benefits, including improved anchors of the prosthetic valve 10 to the native valve and enhanced sealing of fluid flow around the prosthetic valve 10 (e.g., paravalvular leakage). The compression applied by the magnets 62, 64 to the native valve leaflet 82 may press the sealing body 11 against the native valve leaflet 82 and enhance the seal provided by the sealing body 11. In examples, one or more of the magnets 62, 64 may be supplemented or substituted with a magnetic responsive material such as ferromagnetic material as desired.

Features of the examples of FIGS. 1-8 may be utilized solely or in combination with any other features disclosed herein.

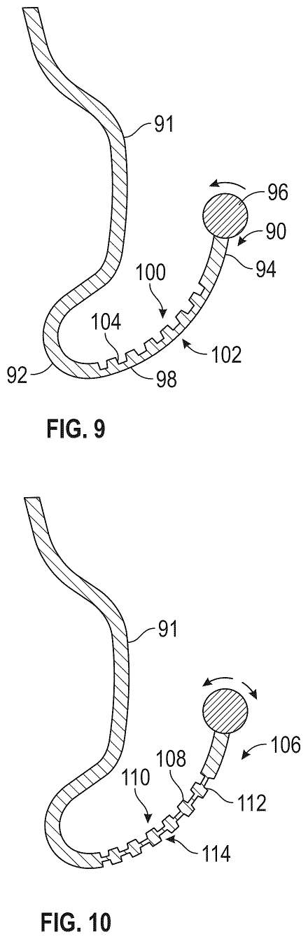

FIG. 9 illustrates another example of an anchor 90 that may be utilized in examples herein. The anchor 90 may be configured to anchor a prosthetic valve and prosthetic valve leaflets to a native valve, which may be in a similar manner as the anchors 17 shown in FIGS. 1-3.

The anchor 90 may comprise an elongate arm that includes a first portion 92 or inner portion and extends radially outward to a second portion 94 or outer portion including a tip 96. The elongate arm has a length. The first portion 92 may be coupled to a valve body 91 and the elongate arm may extend radially outward from the valve body 91 to the tip 96. The valve body 91 may support one or more prosthetic valve leaflets in a similar manner as the valve body shown in FIGS. 1-3 for example.

The anchor 90 may be in a hooked configuration and may include a curved portion 98 having a convex curvature, with a surface 100 facing radially inward and a surface 102 facing radially outward. The anchor 90 may be configured to hook over a distal tip of a native valve leaflet in a similar manner as the anchors 17 shown in FIGS. 1-3.

The anchor 90 preferably includes one or more grooves 104 that are configured to increase the flexibility (i.e., reduce the bending stiffness) of the anchor 90. The one or more grooves 104 may extend transversely to the length of the anchor 90. In examples, a plurality of the grooves 104 may be provided that may be spaced from each other along the length of the anchor 90. As shown in FIG. 9, the one or more grooves 104 may be positioned on the curved portion 98 and may be positioned on the surface 100 facing radially inward.

The one or more grooves 104 are configured to increase a flexibility of the anchor 90 in a direction radially inwardly. This configuration may be used to maintain stiffness in the outward direction while increasing flexibility in the inward direction. As represented by the arrow shown in FIG. 9, the anchor 90 may be configured to deflect radially inwardly upon implantation. Such flexibility may provide a variety of benefits, which may include a reduced electrical conduction disturbance with the native valve annulus and a reduced possibility of damage to the native valve. Enhanced deployment, coupling, and conformability within the native valve may result.

FIG. 10 illustrates an example of an anchor 106 formed with grooves on two sides. More specifically, the anchor includes one or more grooves 108 on an inward facing surface 110 and one or more grooves 112 on an outward facing surface 114. The grooves 112 on the outward facing surface 114 may be configured similarly as the grooves 108 on the inward facing surface 110, which may be similar to the grooves 104. Enhanced flexibility in a radially inward direction and a radially outward direction may result.

The anchors accordingly may be uni-directional, or bi-directional, or may have other directions of flexibility in examples. The enhanced flexibility of the anchors may enhance the possibility of remodeling of the ventricle and may reduce the possibility of conduction disturbance in a ventricle. The anchors may flex as the ventricle remodels.

Features of the examples of FIGS. 9 and 10 may be utilized solely or in combination with any other features disclosed herein.

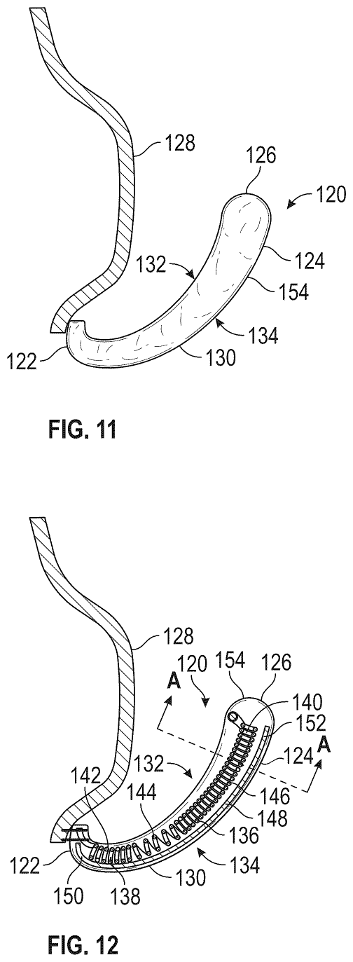

FIGS. 11-13 illustrate another example of an anchor 120 that may be utilized in examples herein. The anchor 120 is adapted to help secure a prosthetic valve within a native valve in a similar manner as the anchors 17 shown in FIGS. 1-3.

The anchor 120 may comprise an elongate arm that may include a first portion 122, inner portion, or coupling portion, and may extend radially outward to a second portion 124 or outer portion including a tip portion 126. The elongate arm may have a length. The first portion 122 may be for coupling to a valve body 128 and the elongate arm may extend radially outwardly from the valve body 128 to the tip portion 126. The valve body 128 may support one or more prosthetic valve leaflets in a similar manner as the valve body shown in FIGS. 1-3 for example.

The anchor 120 may be in a hooked configuration and may include a curved portion 130 having a convex curvature, with a surface 132 facing radially inward and a surface 134 facing radially outward. The anchor 120 may be configured to hook over a distal tip of a native valve leaflet in a similar manner as the anchors 17 shown in FIGS. 1-3. The surface 132 facing radially inward may comprise an inner radius of the anchor 120 and the surface 134 facing radially outward may comprise an outer radius of the anchor 120.

FIG. 12 illustrates a cross-sectional view of the anchor 120 shown in FIG. 11. The illustrated anchor 120 includes a coil 136. The coil 136 may include a first portion 138, inner portion, or coupling portion and may extend radially outward to a second portion 140. The coil 136 may form an elongate arm and may extend from the coupling portion of the anchor 120 radially outward to the tip portion 126 of the anchor. The coil may be integral with the coupling portion or may be a separate component attached thereto.

The coil 136 may comprise a spring coil and may comprise a wire bent into a helical shape. In examples, the coil 136 may comprise a hypotube cut into a coil configuration or may have another configuration as desired.

The coil 136 may have a first coil portion 142 that may have a greater stiffness than a second coil portion 144. The first coil portion 142, for example, may comprise a closer winding of the coil than a second coil portion 144, with the second coil portion 144 having a greater flexibility than the first coil portion 142. The second coil portion 144 may include gaps that allow for local compression and full bending of the coil at the second coil portion 144. The second coil portion 144 may be positioned in a variety of locations, including positioned radially outward of the first coil portion 142 as shown in FIG. 12. In examples, the second coil portion 144 may be positioned radially inward of a first coil portion 142 having a greater stiffness. In examples, multiple portions of the coil 136 may have a greater or lesser stiffness and may be variably positioned as desired. For example, portions having a lesser stiffness may be interleaved with portions having a greater stiffness as desired.

In examples, a second coil portion 144 having a lesser stiffness may be positioned along the curved portion 130 of the anchor 120 to enhance a flexibility of the anchor 120 at a vertex or angled portion of the anchor 120. The anchor 120 accordingly may be able to deflect more easily at the second coil portion 144 than at the first coil portion 142.

In examples, a third coil portion 146 may be positioned radially outward of the second coil portion 144. The third coil portion 146 may have a greater stiffness than the second coil portion 144 and may extend radially outward to the tip portion 126 of the anchor 120. In examples, the second coil portion 144 may be positioned between the first coil portion 142 and the third coil portion 146 to provide greater flexibility at a vertex or angled portion of the anchor 120 between the stiffer first and third coil portions 142, 146. As such, the anchor 120 may have greater flexibility at the second coil portion 144.

In various examples, the anchors 120 may further comprise a stiffener arm 148. The stiffener arm 148 includes an elongate arm that extends along the coil 136 and may increase a stiffness of the anchor 120. The stiffener arm 148 may include a first portion 150 or inner portion and may extend radially outwardly to a second portion 152 or outer portion. The length of the stiffener arm 148 may extend along the length of the coil 136 from the first portion 138 of the coil 136 to the second portion 140 of the coil 136 and may extend along the length of the anchor 120 from the first portion 122 of the anchor 120 to the second portion 124 of the anchor 120.

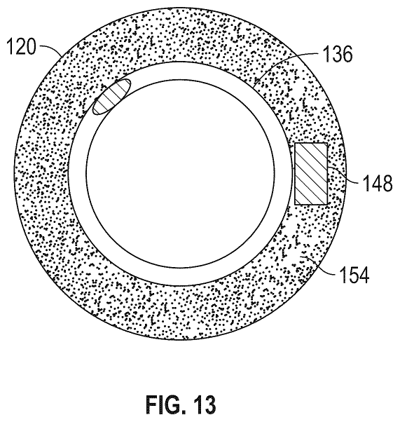

The stiffener arm 148 may extend parallel with the length of the coil 136 and may extend along the outer radius of the anchor 120. The stiffener arm 148 may be positioned outside of the coil 136. The stiffener arm 148 may be circumferentially positioned relative to the coil 136 on one side of the coil 136 and along the outer radius outward of the coil 136. FIG. 13, for example, illustrates a cross sectional view of the anchor 120 along line A-A in FIG. 12. FIG. 13 shows the stiffener arm 148 on one side of the coil 136 and along the outer radius of the coil 136. One or more stiffener arms may be provided in other positions as desired. For example, a stiffener arm may be positioned along the inner radius of the anchor 120, or along the neutral axes of the coil 136 in examples. Multiple stiffener arms may be utilized in various positions as desired.

The stiffener arm 148 may strengthen the anchor 120 and support the anchor 120 from circumferential loads (e.g., forces into and out of the page shown in FIG. 12). The stiffener arm 148 may further reduce the possibility of the anchor 120 deflecting radially outward from the valve body undesirably. In examples, the stiffener arm may be excluded as desired.

In examples, the anchor 120 may include a cover 154 that may be positioned over the coil 136 and the stiffener arm 148. The cover 154 may protect the interior of the anchor 120 comprising the coil 136 and the stiffener arm 148. The cover 154 may further couple the interior of the anchor 120 together, including the coil 136 and the stiffener arm 148. The cover 154 may comprise a sheath that may extend over the coil 136 and the stiffener arm 148. The cover 154 may extend along the entire length of the coil 136 and the stiffener arm 148. The cover 154 may be atraumatic to reduce the possibility of injury to the native valve.

In examples, the cover 154 may comprise an overmold that may be positioned over the coil 136 and the stiffener arm 148. The overmold may be flexible, to allow the anchor 120 to deflect, yet may seal and couple the coil 136 and the stiffener arm 148 together. FIG. 13, for example, illustrates a cover 154 in the form of the overmold encapsulating the coil 136 and the stiffener arm 148. Various forms of overmold may be provided, including a polymer overmold or another form of overmold.

The coil 136 may be configured to increase the flexibility of the anchor 120 in a direction radially inwardly. The flexibility of the anchor 120 may allow the anchor 120 to deflect and conform to the shape of the native valve and may reduce the possibility of electrical conduction disturbance and damage to the native valve. Enhanced deployment, coupling, and conformability within the native valve may result. Enhanced flexibility in a direction radially outward may be provided in examples.

The features of the examples of FIGS. 9-13 may be utilized solely or in combination with any other example disclosed herein.

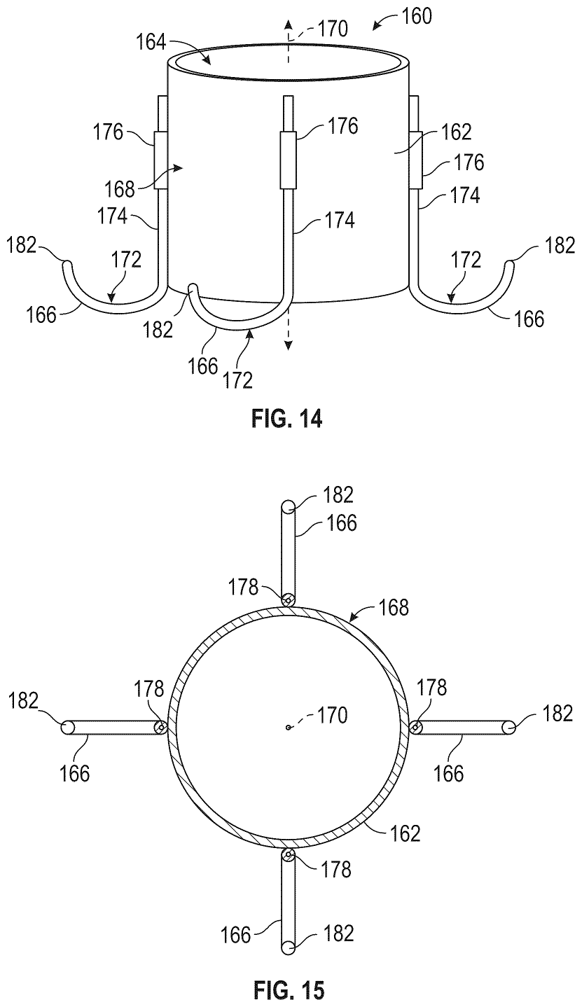

FIG. 14 illustrates another example of a prosthetic valve 160. In this embodiment, the prosthetic valve 160 includes a valve body 162 surrounding an interior flow channel 164. One or more anchors 166 are pivotally coupled to the valve body 162 and are configured to pivot (rotate) about the longitudinal dimension for varying a radial distance of the one or more anchors 166 from the valve body 162.

Similar to earlier embodiments, the valve body 162 supports one or more prosthetic valve leaflets (as shown in FIG. 2 for example) positioned within the flow channel 164. The valve body 162 may comprise a frame and may comprise a sealing body, which may be similar to other examples disclosed herein. An outer surface 168 of the valve body 162, for example, may comprise a portion of the valve body 162 configured to contact a portion of a native valve that may include native valve leaflets or another portion of the valve body 162. The outer surface 168 of the valve body 162 for example may comprise a sealing skirt that may be configured to seal against a portion of a native valve. Various other configurations of valve bodies may be utilized in examples.

The longitudinal dimension of the flow channel 164 defines a direction of flow of fluid through the prosthetic valve 160. A central axis 170 (marked in FIGS. 14 and 15) may extend along the longitudinal dimension. The valve body 162 may be centered upon the central axis 170.

The anchors 166 may comprise distal anchors as shown in FIG. 14. The distal anchors, for example, may each have a hooked configuration. The distal anchors, for example, may have a curved portion 172 that may be configured to hook over the distal tips of native valve leaflets to anchor the prosthetic valve 160 to an implantation site. Various other configurations of anchors 166 may be utilized in examples.

The anchors 166 may include an inner portion or coupling portion 174 that may pivotally couple the anchors 166 to the valve body 162. A coupler in the form of a hinge 176 or other form of coupler may couple the coupling portion 174 to the valve body 162. The hinge 176 secures the anchors to the valve body while allowing the anchors to rotate relative to the hinge (or other similar mechanism). Each anchor 166, for example, may be configured to pivot about a respective axis 178 (marked in FIG. 15) that may extend parallel with the central axis 170. Each anchor 166 may be circumferentially spaced from each other, and the spacing may be equal or varied in examples.

The anchors 166 may each include a tip 182 configured to rotate about the hinge 176.

The pivoting of the anchors 166 may vary the maximize radial distance of the anchor 166 from the valve body 162. For example, referring to FIG. 15, the anchors 166 are shown in a top view and extending perpendicular to the outer surface 168 of the valve body 162. The anchors 166 may have their greatest radial extent from the outer surface 168 of the valve body 162 in such a configuration.

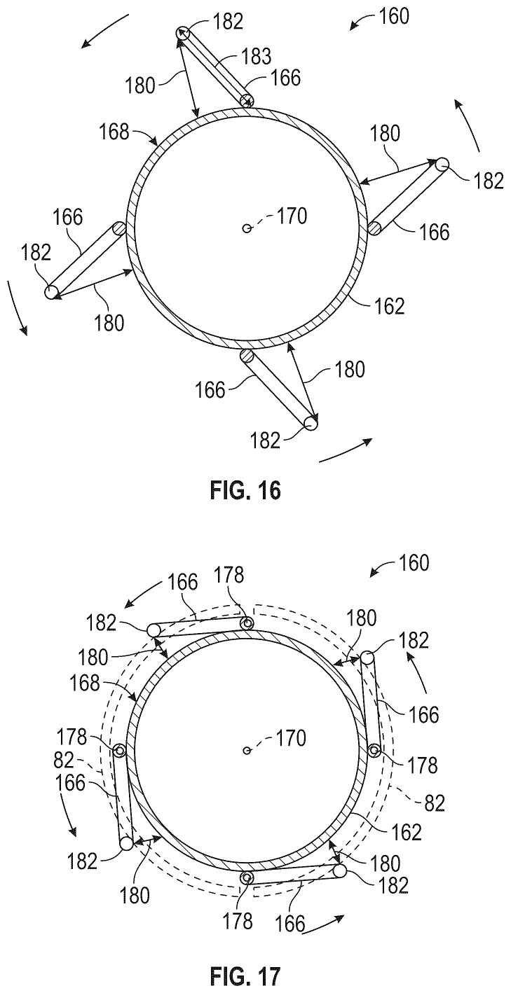

Referring to FIG. 16, the anchors 166 may pivot about the longitudinal dimension to vary the radial distance 180 of the anchors 166 from the valve body 162. Each anchor 166, as shown in FIG. 16 has rotated in a counter-clockwise direction to draw the tips 182 of the anchors 166 towards the outer surface 168 of the valve body 162. The greatest radial extent of the anchors 166 has reduced from the position shown in FIG. 15. The greatest radial extent of the prosthetic valve 160 has varied. The anchors 166 may continue to pivot as desired. A length 183 of each of the anchors 166 may remain constant upon the anchor 166 pivoting about the longitudinal dimension.

FIG. 17, for example, illustrates the anchors 166 continuing to rotate in the counter-clockwise direction. The tips 182 may be drawn towards the outer surface 168 of the valve body 162 and the greatest radial extent of the anchors 166 has reduced. At least a portion of the anchors 166 may extend parallel with the outer surface 168 of the valve body 162.

The anchors 166 may pivot to conform to a size of a native valve. As such, if the native valve has a smaller size, the anchors 166 may have a reduced greatest radial extent and if the native valve has a larger size, the anchors 166 may have an increased greatest radial extent. The angle that the anchors 166 are positioned at relative to the outer surface 168 of the valve body 162 accordingly may vary to conform to the size of the native valve. FIG. 17, for example, illustrates in dashed lines the representative position of the native valve leaflets 82. The tips 182 of the anchors 166 may be positioned radially outward of the native valve leaflets 82. If the native valve leaflets 82 were thicker or positioned further from the outer surface 168 of the valve body 162 then the anchors 166 accordingly may be rotated further from the outer surface 168 of the valve body 162 and may have a greater radial extent than shown in FIG. 17.

The anchors 166 in examples, may be configured to press a native valve leaflet to an outer surface 168 of the valve body 162. The outer surface 168 may comprise a sealing body, such as a sealing skirt or other form of sealing body as desired. As such, the anchors 166 may improve a fluid seal between the valve body 162 and the native valve and may reduce leakage such as paravalvular leakage in examples.

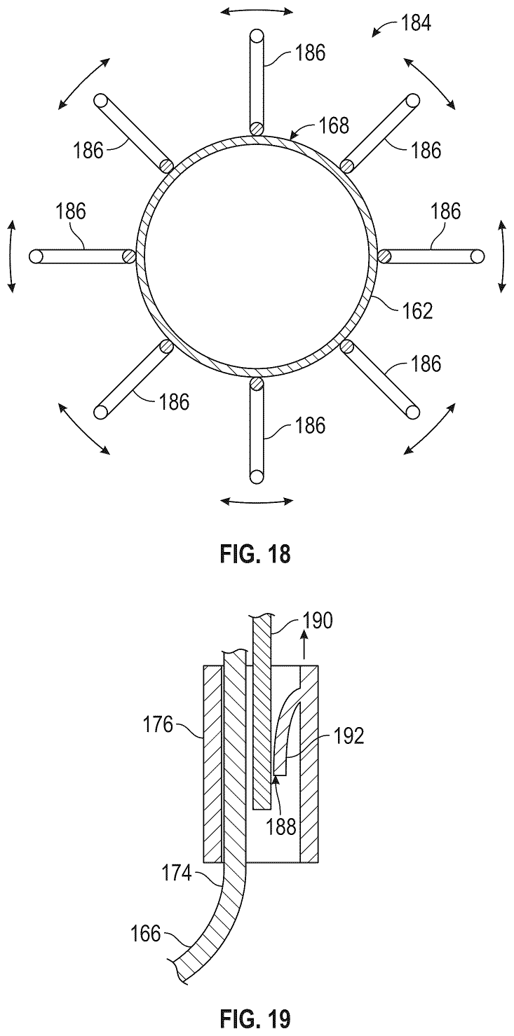

Varied numbers of anchors 166 may be utilized in examples. FIG. 18, for example, illustrates an example of a prosthetic valve 184 including a greater number of anchors 186 than shown in the example of FIGS. 14-17. The plurality of anchors 186 may be circumferentially spaced from each other, and the circumferential spacing may be equal or may be varied in examples.

The anchors 166, 186 may be deflected to a radial position prior to insertion and deployment within a patient's body. For example, the native valve may be sized prior to insertion of the prosthetic valves 160, 184 and the anchors 166, 186 accordingly may be set to a defined angle prior to insertion. The prosthetic valves 160, 184 may be crimped to a delivery apparatus with the anchors 166, 186 set at the radial position and then deployed to the native valve, which may include deployment methods disclosed herein.

In examples, the anchors 166, 186 may be able to pivot in vivo. Upon deployment, the anchors 166, 186 and/or the valve body 162 may be rotated to the greatest radial distance of the anchors 166, 186 from the valve body 162. The anchors 166, 186, for example, may be deployed from a delivery apparatus or hooked around the native valve leaflets and then pivoted to vary the radial distance from the valve body 162. The variation of the greatest radial distance may occur in vivo and may occur in combination with imaging of the implantation site.

In examples, the valve body 162 may be rotated by a delivery apparatus relative to the native valve to vary the greatest radial distance of the anchors 166, 186 from the valve body 162.

In examples, the anchors 166, 186 may be freely able to pivot relative to the valve body 162. As such, the anchors 166, 186 upon deployment may be able to pivot about the valve body 162 freely to conform to the size of the native valve. The anchors 166, 186 may be able to pivot to allow the anchoring site to grow or shrink as desired. This feature may be used to actively alter a diameter of the native valve during deployment.

In examples, one or more locks may be provided that may lock the anchors 166, 186 in position relative to the valve body 162. The lock may lock movement of the anchors 166, 186 about the longitudinal dimension. The lock may be set prior to insertion of the prosthetic valves 160, 184 into the patient's body. In examples, the lock may be actuated in vivo.

FIG. 19, for example, illustrates a cross sectional view of the hinge 176 that may include a lock 188. The lock 188 may include a spacer 190 and a protrusion 192 or may have another configuration for locking as desired. The lock 188 may be configured to be actuated in vivo. For example, the coupling portion 174 of the anchor 166 may pass through the hinge 176 and may be configured to pivot within the hinge 176. Upon the anchor 166 pivoting to the desired position, a spacer 190 may be withdrawn from the hinge 176 to allow the protrusion 192 to contact the coupling portion 174 of the anchor 166 to lock the anchor 166 in position. The spacer 190 may be coupled to a delivery apparatus that may be configured to withdraw the spacer 190 from the hinge 176.

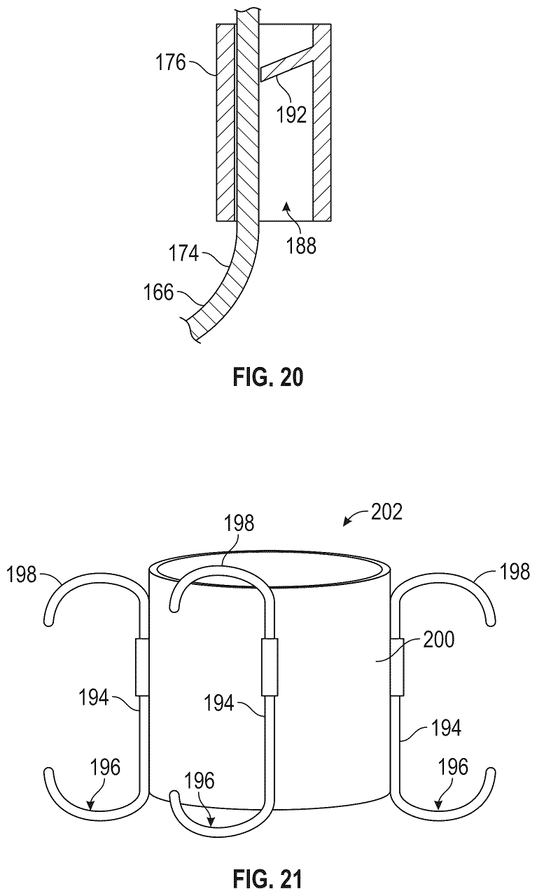

FIG. 20, for example, illustrates the spacer 190 having been withdrawn from the hinge 176 to lock the anchor 166 in position. The lock 188 may be locked in vivo, upon the anchors 166 being placed in the desired angle with respect to the valve body 162. Various other configurations of locks may be utilized in examples as desired.

The anchors 166, 186 may have various shapes and configurations. FIG. 21, for example, illustrates anchors 194 having a distal curved portion 196, similar to the curved portion 172 shown in FIG. 14, which may be configured to hook over distal tips of prosthetic valve leaflets. The anchors 194 may include distal anchors or ventricular anchors. The anchors 194 may each include a proximal frame support 198 that may be configured to support a sealing body or may be utilized as a proximal or atrial anchor as desired. The proximal frame support 198, for example, may comprise an elongate arm that may extend radially outward from the valve body 200. The proximal frame support 198 may have a hooked configuration and may be configured to hook over a proximal or atrial side of the native valve to anchor the prosthetic valve 202 in position. The proximal frame support 198 may have other configurations in examples as desired. The proximal frame support 198 may be coupled to the respective distal anchor and configured to pivot about the longitudinal dimension with the distal anchor. The proximal frame support 198 may be integral with the distal curved portion 196 in examples. Various other configurations of anchors 194 may be utilized as desired.

In examples, the anchors 166, 186, 194 may be configured to be adjusted in position axially. For example, the axial position of the anchors 166, 186, 194 along a respective axis that the anchor may pivot about (e.g., an axis 178 as marked in FIG. 15 for example) may be adjusted.

The adjustment may be along the dimension of the flow channel of the prosthetic valve (e.g., parallel with the central axis 170 marked in FIG. 15). The adjustment may be in a proximal direction or in a distal direction as desired (or combinations of proximal and distal directions). The adjustment may occur prior to implantation or in vivo. An in vivo adjustment may allow for the axial position of the anchors 166, 186, 194 to be adjusted to address a size of the native anatomy (e.g., a size or length of a native leaflet). An in vivo adjustment may be utilized to allow for capture or recapture of a leaflet that an anchor has missed capture of. For example, an anchor that misses capture of a leaflet may be advanced distally to allow an anchor to extend over a native valve leaflet and may then be retracted proximally to allow for capture of the leaflet. Combinations of axial position adjustment and variations of the radial distance of anchors 166, 186, 194 from a valve body may be utilized in examples.

The features of the examples of FIGS. 14-21 may be utilized solely or in combination with any other example disclosed herein.

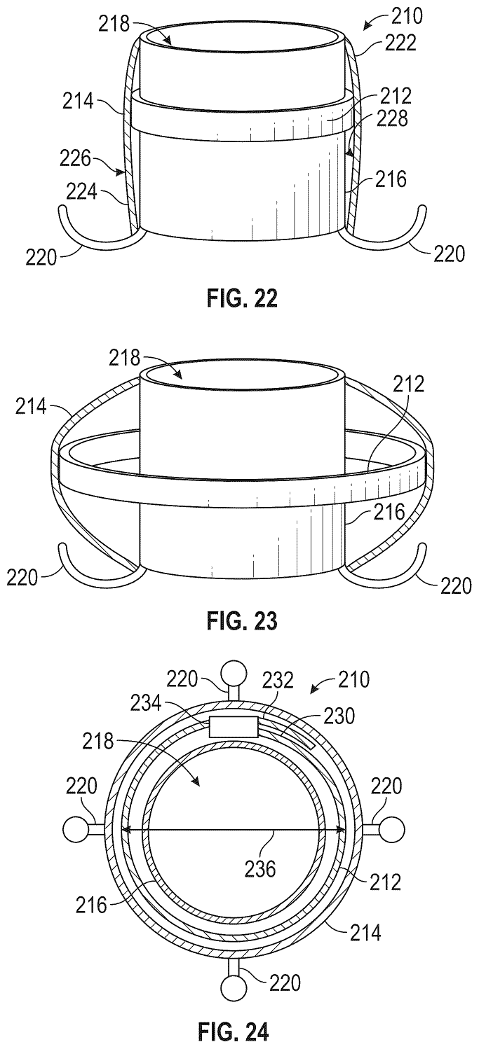

FIG. 22 illustrates another example of a prosthetic valve 210. In this embodiment, the prosthetic valve 210 includes a band 212 having an adjustable diameter, thereby allowing a sealing body 214 to be adjusted radially inwardly or outwardly. The prosthetic valve 210 may include one or more prosthetic valve leaflets (as shown in FIG. 2 for example) positioned within a flow channel 218 for providing a one-way valve. The valve body 216 may surround the flow channel 218 and may include a frame that may support the prosthetic valve leaflets within the flow channel 218. The frame may comprise an inner frame that may be positioned radially inward of the sealing body 214. The valve body 216 may have a cylindrical shape or other shape as desired.

The prosthetic valve 210 may include one or more anchors 220 that may be configured to anchor the one or more prosthetic valve leaflets to a native valve. The anchors 220 may comprise distal anchors that may be configured similarly as the anchors 17 shown in FIG. 1 for example. The anchors 220, for example, may have a hooked configuration. The distal anchors, for example, may be configured to hook over the distal tips of native valve leaflets to anchor the prosthetic valve 210 to an implantation site. Various other configurations of anchors 220 may be utilized in examples.

A sealing body 214 may be positioned radially outwardly of the one or more prosthetic valve leaflets and may be configured to seal against a portion of the native valve. The sealing body 214 may include a proximal portion 222 that may be coupled to a proximal portion of the valve body 216 and may include a distal portion 224 that may be coupled to a distal portion of the valve body 216 or to the one or more anchors 220. The sealing body 214 includes an outer surface 226 configured to contact to and seal against surrounding tissue of the native valve. The sealing body 214 may include an inner surface 228 that may face opposite the outer surface 226.

The sealing body 214, in examples, may be flexible and may be configured to be elastic. The sealing body 214 accordingly may be configured to move or stretch radially outward from the valve body 216 if desired. The sealing body 214 may comprise a sealing skirt.

The band 212 extends around the valve body 216 and around the prosthetic valve leaflets. The band 212 may be positioned radially outward of the valve body 216 and the prosthetic valve leaflets. The band 212 may extend circumferentially about the sealing body 214. For example, the band 212 may be positioned radially inward of the sealing body 214 in examples, or may be positioned radially outwardly of the sealing body 214, or may be positioned within the sealing body 214 in examples. The band 212 may form a loop in examples. The loop may be configured to apply a force against the sealing body 214 to move the sealing body 214 radially outward or inward.

FIG. 24 illustrates a top cross sectional view of the prosthetic valve 210 shown in FIG. 22. The band 212 may have a first end portion 230 and a second end portion 232. The first end portion 230 and the second end portion 232 may be coupled together in examples or may be free from each other. For example, as shown in FIG. 24, the first end portion 230 may include a coupler 234 that the second end portion 232 may slide through. The coupler 234 may be positioned at the end of the first end portion 230 and the second end portion 232 may be configured to slide through the coupler 234 to adjust the diameter 236 of the band 212. The first end portion 230 may extend along a length of the second end portion 232. The band 212 may comprise a loop having an end at the second end portion 232 and a first end portion 230 configured to overlap the end at the second end portion 232. The band 212 may have a circular shape. Other configurations may be utilized in examples.

The amount that the first end portion 230 overlaps the second end portion 232 may be varied to vary the diameter 236 of the band 212. For example, a reduced amount of overlap may increase the diameter 236 of the band 212 and a greater amount of overlap may reduce the diameter 236. Upon the diameter 236 of the band 212 being increased, the band 212 may move the sealing body 214 radially outward. Upon the diameter 236 of the band 212 being decreased, the band 212 may move the sealing body 214 radially inward.

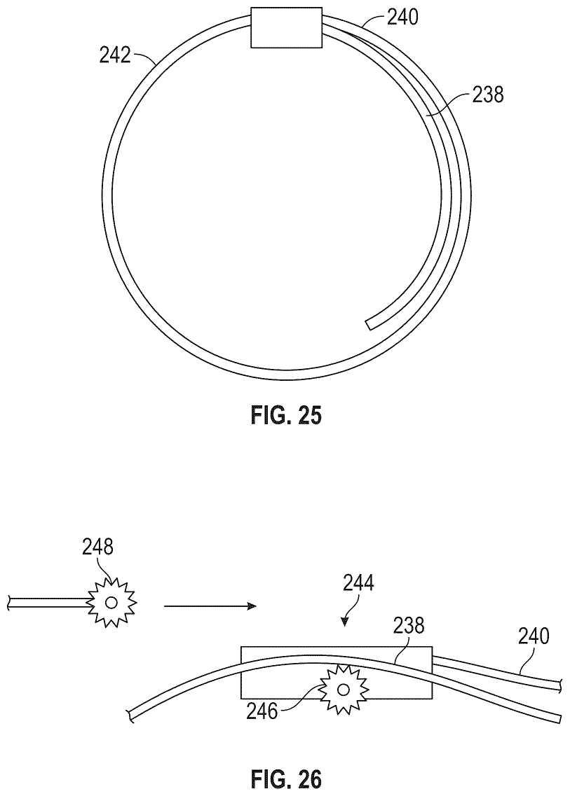

The second end portion 232 of the band 212 may be configured to be positioned radially outward of the first end portion 230 as shown in FIG. 24. In examples, a second end portion 238 may be positioned radially inward of a first end portion 240 of a band 242, as shown in FIG. 25 for example.

The diameter 236 of the band 212 may be adjusted in a variety of manners. For example, a portion of the band 212 may be accessible through the sealing body 214 and manipulated to vary the diameter 236 of the band 212. Referring to FIG. 24, the second end portion 232 may be gripped, for example, and slid relative to the first end portion 230 to vary the diameter 236 of the band 212. The second end portion 232 may be gripped with a device such as forceps or a portion of the delivery system as desired. The coupler 234 may include a ratcheting lock or other form of lock that may maintain the diameter 236 of the band 212 as desired.

In examples, an adjustment mechanism may be provided that may be configured to adjust the diameter of the band. For example, referring to FIG. 26, an adjustment mechanism 244 may include a gear 246 that may be configured to be rotated to adjust the amount of overlap of the first end portion 240 relative to the second end portion 238. The gear 246, for example, may include teeth that may engage recesses on the second end portion 238, and may be configured to rotate to move the second end portion 238 relative to the first end portion 240. An adjustment device comprising a mating gear 248 may be used to engage the gear 246. The mating gear 248, for example, may be powered or otherwise rotatable (e.g., via a pull tether or another control), to rotate the mating gear 248. The mating gear 248 may contact the gear 246 and may be rotated in a direction to either increase or decrease the diameter of the band. The adjustment mechanism may be positioned at an end of the band and configured to adjust the diameter of the band. Other forms of adjustment mechanisms may be utilized as desired.

The band may be utilized to vary the outward extent of the sealing body 214 to allow the sealing body 214 to press against and conform to the shape of a native valve. As such, the prosthetic valve 210 may be deployed and the band 212 may be adjusted in vivo to cause the sealing body 214 to contact and apply a force to the native valve. The contact between the sealing body 214 and the native valve may reduce fluid flow or leakage around the prosthetic valve 210. The band may be adjusted in vivo to account for the varied size or shape of the native valve.

In examples, the diameter of the band may be adjusted prior to insertion into a patient's body. For example, a size of the native valve may be imaged and the band may be adjusted to conform to the size of the native valve.

The features of the examples of FIGS. 22-26 may be utilized solely or in combination with any other example disclosed herein.

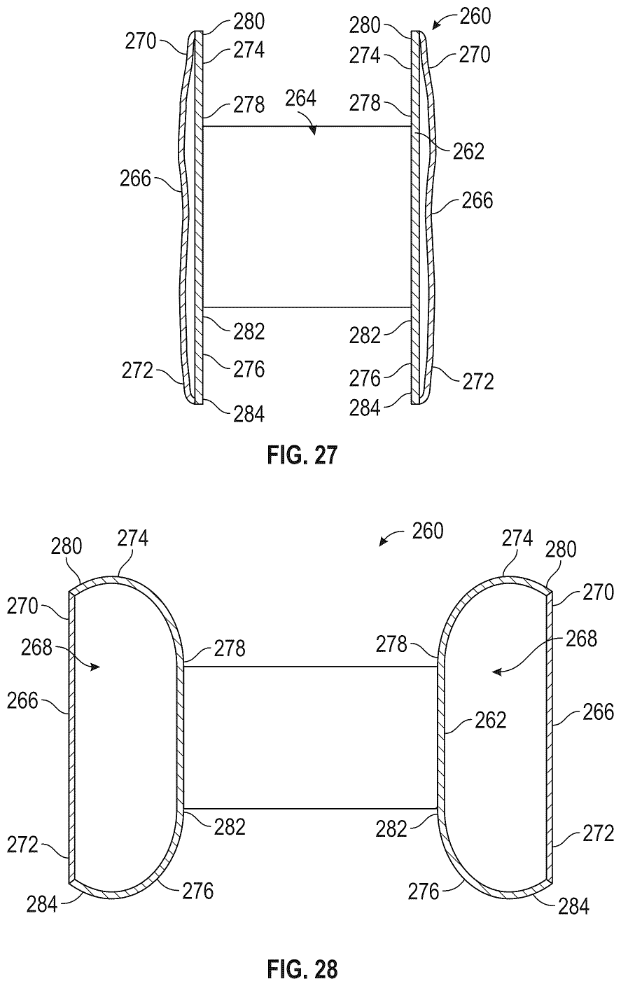

FIG. 27 illustrates another example of a prosthetic valve 260 including a valve frame 262 surrounding a flow channel 264. A sealing skirt 266 is positioned radially outwardly from the valve frame 262, wherein the sealing skirt 266 is held in tension and is adapted to deflect to conform to a shape of the native valve. FIG. 28 illustrates a space (e.g., clearance) 268 between the sealing skirt 266 and the valve frame 262.

Referring to FIG. 27, the valve frame 262 may support one or more prosthetic valve leaflets (as shown in FIG. 2 for example) coupled to the valve frame 262 that may extend radially inward from the valve frame 262 and may be positioned within the flow channel 264. The valve frame 262 may support the prosthetic valve leaflets within the flow channel 264. The valve frame 262 may be positioned radially inward of the sealing skirt 266. The valve frame 262 may have a cylindrical shape or other shape as desired.

The valve frame 262 may be configured to compress and expand in a similar manner as the inner frame 18 discussed with respect to FIGS. 1 through 3. The valve frame 262, for example, may expand radially outward and the length of the valve frame 262 may reduce (as shown in FIG. 28, for example). The valve frame 262 may be in a compressed or undeployed configuration in FIG. 27 and may be in an uncompressed or deployed configuration in FIG. 28. As such, the diameter of the valve frame 262 may expand radially outwardly and the length of the valve frame 262 may decrease as shown in FIG. 28.

The sealing skirt 266 may be positioned radially outwardly of the one or more prosthetic valve leaflets and the valve frame 262 and may be configured to seal against a portion of the native valve. The sealing skirt 266 may surround the valve frame 262. The sealing skirt 266 may include a proximal portion 270 and a distal portion 272. The sealing skirt 266 may be suspended between the proximal portion 270 and the distal portion 272 and may be held with a space 268 between sealing skirt 266 and the valve frame 262.

The prosthetic valve 260 may include one or more proximal tensioning bodies 274 and may include one or more distal tensioning bodies 276. The proximal tensioning bodies 274 may be positioned at a proximal portion of the prosthetic valve 260 and the distal tensioning bodies 276 may be positioned at a distal portion of the prosthetic valve 260. The proximal portion 270 of the sealing skirt 266 including the proximal end of the sealing skirt 266 may be coupled to the proximal tensioning bodies 274. The distal portion 272 of the sealing skirt 266 including the distal end of the sealing skirt 266 may be coupled to the distal tensioning bodies 276.

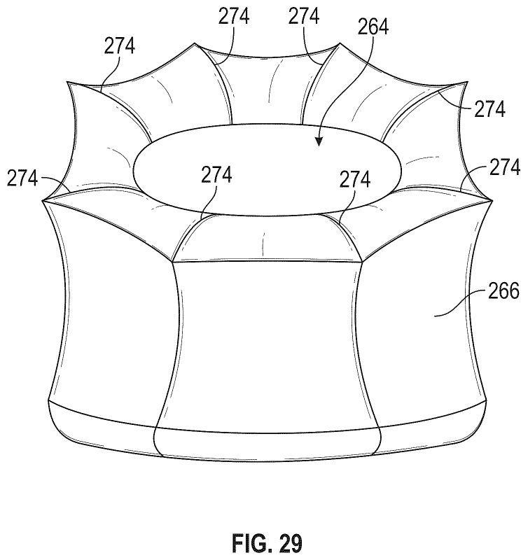

The proximal tensioning bodies 274 may have a variety of forms. As shown in FIGS. 27-29, the proximal tensioning bodies 274 may comprise arms having a first end 278 coupled to a proximal portion of the valve frame 262 and a second end 280 configured to extend radially outward from the first end 278. The proximal tensioning bodies 274 may extend radially outward from the proximal portion of the valve frame 262. Similarly, the distal tensioning bodies 276 may comprise arms having a first end 282 coupled to a distal portion of the valve frame 262 and a second end 284 configured to extend radially outward from the first end 282. The distal tensioning bodies 276 may extend radially outward from the distal portion of the valve frame 262. Referring to FIG. 29, the arms of the proximal tensioning bodies 274 and the distal tensioning bodies may be spaced circumferentially from each other and may extend radially outward from the flow channel 264. The spacing may be circumferentially equidistant from each other or varied spacing may be provided.

The sealing skirt 266 may be tensioned by the proximal tensioning body 274 and the distal tensioning body 276. For example, referring to FIGS. 28 and 29, upon expansion of the valve frame 262, the sealing skirt 266 may be tensioned and held in suspension between the proximal and distal tensioning bodies 274, 276. A central portion of the sealing skirt 266 may be uncoupled and free from a frame and able to deflect inward to conform to a shape of a native valve. The sealing skirt 266, for example, may be elastic and configured to deflect inward to conform to the shape of the native valve.

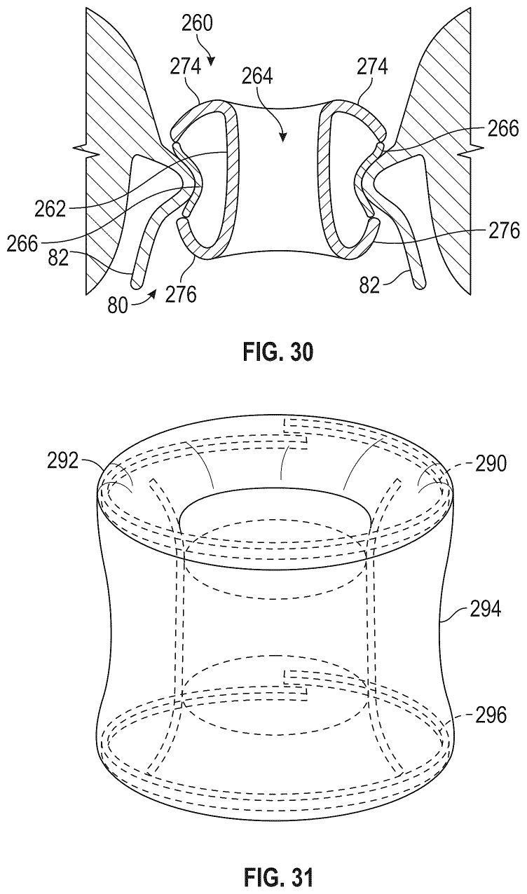

FIG. 30, for example, illustrates the prosthetic valve 260 deployed to a native valve 80. The tensioned sealing skirt 266 deflects radially inward to conform to the shape of the native valve 80 and particularly the position of the native valve leaflets 82. The sealing skirt 266 may form a seal with the native valve 80 to reduce fluid flow outside of the flow channel 264 (e.g., paravalvular leakage).

As shown in FIG. 30, the distal tensioning bodies 276 in examples may comprise a distal or ventricular anchor for the prosthetic valve 260. The proximal tensioning bodies 274 in examples may comprise a proximal or atrial anchor for the prosthetic valve 260. The distal and proximal tensioning bodies 276, 274 accordingly may anchor the prosthetic valve 260 in position and impede proximal and distal movement, or atrial and ventricular movement.

The proximal or distal tensioning bodies may have other forms in examples. For example, referring to FIG. 31, a proximal tensioning body may comprise a ring 290. The ring 290 may be compressible and biased to expand outward or to a shape as shown in FIG. 31. The ring 290 may support the proximal end portion 292 of the sealing skirt 294. A distal tensioning body may comprise a ring 296. The ring 296 may similarly be compressible and biased to expand outward or to a shape as shown in FIG. 31.

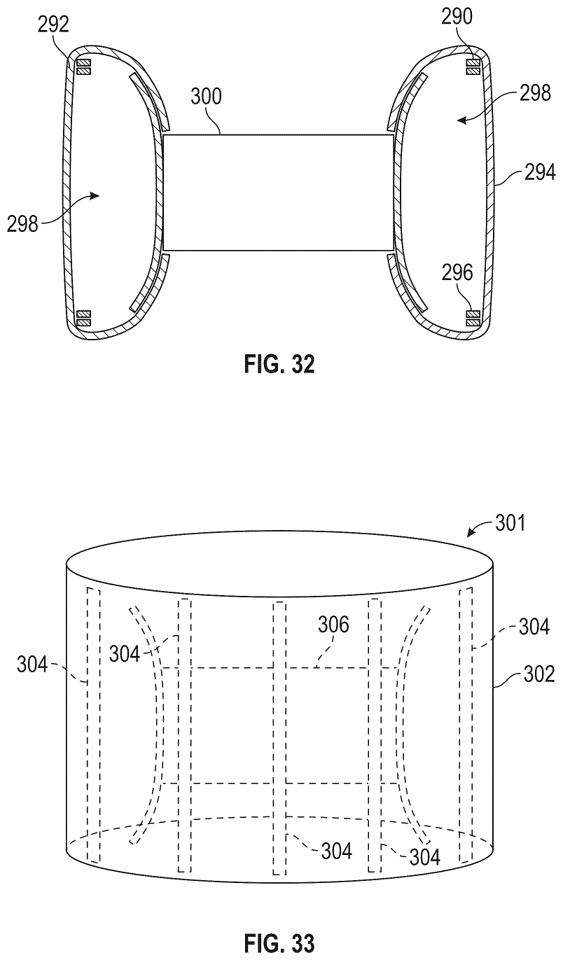

The sealing skirt 294 may be tensioned by the proximal ring 290 and the distal ring 296. For example, as shown in the cross sectional view of FIG. 32, the sealing skirt 294 may be in tension and positioned with a space 298 between the sealing skirt 294 and the valve frame 300. The sealing skirt 294 may be configured to deflect inward in a similar manner as the sealing skirt 266 shown in FIG. 30.

FIG. 33 illustrates a prosthetic valve 301 in which the sealing skirt 302 is supported by a plurality of wires 304. The wires 304 may extend along the sealing skirt 302. The sealing skirt 302 may be positioned radially outward from a valve frame 306 with a space between the sealing skirt 302 and the valve frame 306, the sealing skirt 302 being in tension and configured to deflect to conform to a shape of a native valve. The plurality of wires 304 may extend axially and may be circumferentially spaced from each other about the valve frame 306. The plurality of wires 304 may be configured to deflect inward with the sealing skirt 302.

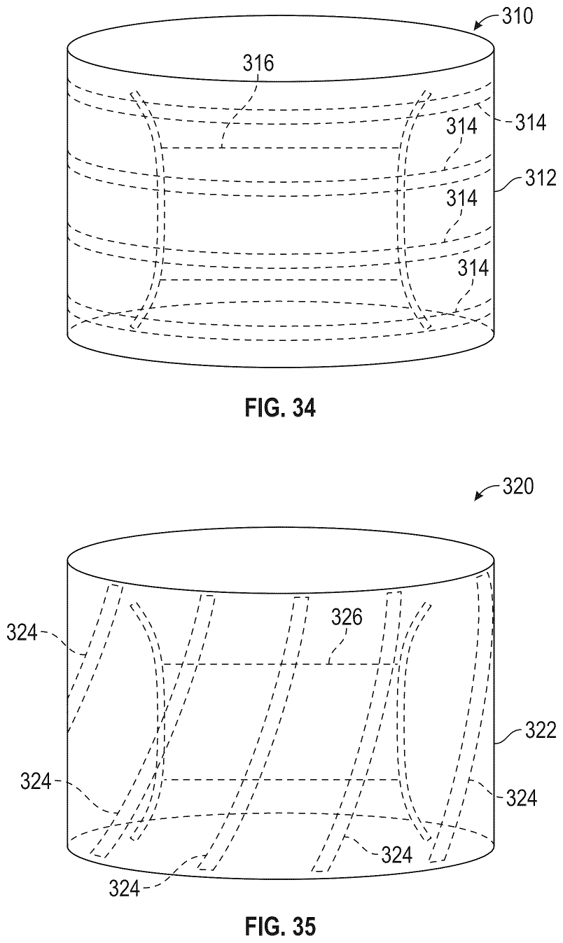

FIG. 34 illustrates an example of a prosthetic valve 310 in which the sealing skirt 312 is supported by a plurality of wires 314. The wires 314 may extend along the sealing skirt 312. The sealing skirt 312 may be positioned radially outward from a valve frame 316 with a space between the sealing skirt 312 and the valve frame 316, the sealing skirt 312 being in tension and configured to deflect to conform to a shape of a native valve. The plurality of wires 314 may each extend circumferentially and may be axially spaced from each other. The plurality of wires 314 may be configured to deflect inward with the sealing skirt 312.

FIG. 35 illustrates an example of a prosthetic valve 320 in which the sealing skirt 322 is supported by a plurality of wires 324. The wires 324 may extend along the sealing skirt 322. The sealing skirt 322 may be positioned radially outward from a valve frame 326 with a space between the sealing skirt 322 and the valve frame 326, the sealing skirt 322 being in tension and configured to deflect to conform to a shape of a native valve. The plurality of wires 324 may each extend axially at an angle with respect to the central axis of the prosthetic valve 320. The plurality of wires 324 may be circumferentially spaced from each other about the valve frame 326. The plurality of wires 324 may be configured to deflect inward with the sealing skirt 322.

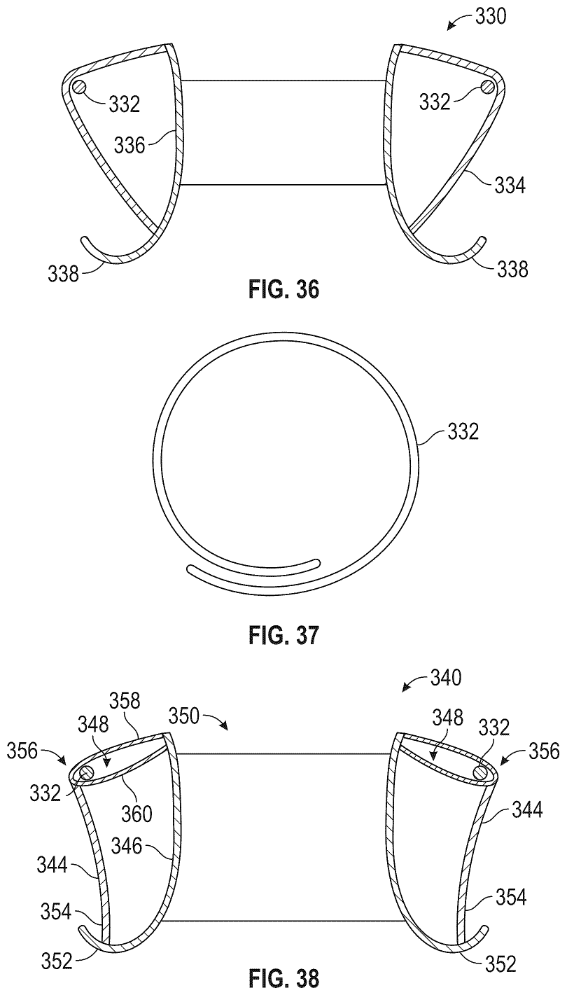

FIG. 36 illustrates an example of a prosthetic valve 330 in which a proximal tensioning body comprises a ring 332 or similar toroidal shaped member. A distal portion of the sealing skirt 334 may be coupled to a portion of the valve frame 336 or to one or more of the distal anchors 338. The sealing skirt 334 may be positioned radially outward from a valve frame 336 with a space between the sealing skirt 334 and the valve frame 336, the sealing skirt 334 being in tension and configured to deflect to conform to a shape of a native valve. The ring 332 may be configured similarly as the ring 290 shown in FIG. 31. FIG. 37, for example, illustrates a top view of the ring 332, which may have overlapping end portions.

In examples, a sealing skirt may include a pocket. A ring or other form of tensioning member may be positioned within the pocket. FIG. 38, for example, illustrates a cross sectional view of a prosthetic valve 340 including a sealing skirt 344 positioned radially outward from the valve frame 346 and including a pocket 348 that extends around the circumference of the valve 340. The ring 332 may be positioned within the pocket 348 and configured to support the sealing skirt 344. The ring 332 may comprise a proximal tensioning body configured to tension the sealing skirt 344.

The valve frame 346 may be configured similarly as the valve frame 262 discussed with respect to FIG. 27. The valve frame 346 may surround a flow channel 350, in which one or more prosthetic valve leaflets may be coupled to the valve frame 346 and may extend radially inward from the valve frame 346 and be positioned within the flow channel 350. The prosthetic valve leaflets may extend radially inward from the valve frame 346 and may be positioned within the flow channel 350. The valve frame 346 may support the prosthetic valve leaflets within the flow channel 350.

One or more distal anchors 352 may be coupled to distal portion of the valve frame 346. The distal anchors 352 may be configured similarly as the distal anchors 17 discussed with respect to FIG. 1. The distal anchors 352, for example, may have a hooked configuration. The distal anchors, for example, may be configured to hook over the distal tips of native valve leaflets to anchor the prosthetic valve 340 to an implantation site. Various other configurations of anchors 352 may be utilized in examples.

The sealing skirt 344 may include a distal portion 354 coupled to the one or more distal anchors 352 and a proximal portion 356 comprising the pocket 348. The proximal portion 356 of the sealing skirt 344 may further be coupled to a proximal portion of the valve frame 346.

The pocket 348 may comprise an overlapping material of the sealing skirt 344. For example, as shown in FIG. 38, the pocket 348 may include a first portion 358 or proximal portion and a second portion 360 or distal portion extending along the length of the first portion 358. The first portion 358 and the second portion 360 of the pocket 348 may extend parallel with each other. The space between the first portion 358 and the second portion 360 may comprise a cavity for receiving the ring 332. The first portion 358, second portion 360, and the ring 332 may each extend circumferentially about the valve frame 346 such that the cavity and the pocket 348 have an annular shape about the valve frame 346. The pocket 348 may serve to position the ring 332 relative to the sealing skirt 344 at a desired location.

The sealing skirt 344 may be in tension and configured to deflect to conform to a shape of a native valve.

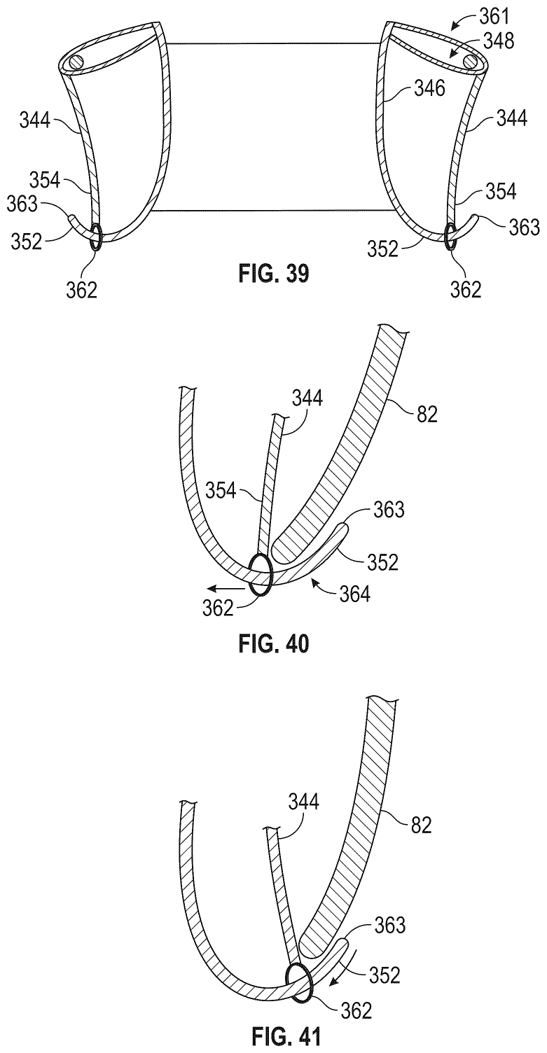

In examples, a distal portion of a sealing skirt may be coupled to one or more slidable couplers, with each slidable coupler configured to slide relative to one or more anchors. FIG. 39, for example, illustrates a prosthetic valve 361 including one or more slidable couplers 362. The one or more slidable couplers 362 may couple the distal portion 354 of the sealing skirt 344 to the one or more distal anchors 352 and may be configured to allow the distal portion 354 of the sealing skirt 344 to slide relative to the distal anchors 352.

The sealing skirt 344 may be configured similarly as the sealing skirt 344 discussed with respect to FIG. 38 and may or may not include the pocket 348 discussed with respect to FIG. 38. The sealing skirt 344 may be positioned radially outward from the valve frame 346. The sealing skirt 344 may be in tension and configured to deflect to conform to a shape of a native valve. The valve frame 346 may be configured similarly as the valve frame 346 discussed with respect to FIG. 38. The valve frame 346 may surround a flow channel. The valve frame 346 may support one or more prosthetic valve leaflets that may extend radially inward from the valve frame 346 and may be positioned within the flow channel.

The distal anchors 352 may be configured similarly as the distal anchors 352 discussed with respect to FIG. 38. The distal anchors 352 may be configured to anchor the prosthetic valve leaflets to the native valve. Each of the distal anchors 352 may comprise an elongate arm. The elongate arm may extend radially outward to a tip 363 of the elongate arm.

Referring to FIG. 40, each of the slidable couplers 362 may comprise a ring. The ring may surround a respective one of the one or more anchors 352 and may be configured to slide along the one or more anchors 352. The slidable couplers 362 may be configured to slide along a length of the one or more anchors 352 in a radially inward direction and may be configured to slide along a length of the one or more anchors 352 in a radially outward direction in examples.

For example, the distal anchors 352 may comprise an elongate arm and the slidable couplers 362 may be configured to slide along the length of the elongate arm. The slidable couplers 362, for example, may slide away from the tip 363, or radially inward, relative to the tip 363 of the elongate arm as marked in FIG. 40. The slidable couplers 362 may slide away from the tip 363 to increase a size of a protruding portion 364 of the elongate arm that is positioned between the slidable coupler 362 and the tip 363. The protruding portion 364 may overlap a distal tip of a native valve leaflet. The size of the protruding portion 364 may be increased to conform to a shape of a native valve.

For example, as shown in FIG. 40, a leaflet 82 may have a greater length or thickness that may cause the slidable coupler 362 to slide radially inward and away from the tip 363. The size of the protruding portion 364 accordingly may increase to accommodate the size of the leaflet 82 between the tip 363 and the sealing skirt 344. The distal portion 354 of the sealing skirt 344 accordingly may slide relative to the anchor 352. The distal portion 354 of the sealing skirt 344 may slide radially inward and may contact the leaflet 82 to form a seal with the leaflet 82.

The movement of the slidable coupler 362 accordingly may accommodate for various configurations of the native valve, to allow for sealing if the native valve has a greater size, as shown in FIG. 40. In examples, the slidable coupler 362 may slide radially inward to a lesser distance if the native valve has a relatively smaller size. FIG. 41, for example, illustrates the slidable coupler 362 sliding radially inward to a lesser distance than shown in FIG. 40. Such a configuration may result from a lesser length or size of the leaflet 82 than shown in FIG. 40. The slidable couplers 362 accordingly may accommodate various sizes of native valves.

In examples, the slidable coupler 362 may be configured to slide radially outward. The movement of the slidable coupler 362 may allow the sealing skirt 344 to be drawn radially outward upon a smaller size of a native valve.

The prosthetic valve 361 may be deployed to a native valve and the slidable couplers 362 may automatically slide to conform to the shape of a native valve. The slidable couplers 362, for example, may slide inward or outward according to the shape of the native valve.

In examples, the slidable couplers 362 may be controlled during or following deployment to adjust the position of the slidable couplers 362. Such adjustment may occur in vivo. The deployment may be imaged and the slidable couplers 362 may be slid to account for the size and shape of the native valve. In examples, the slidable couplers 362 may be adjusted prior to insertion into the patient's body.

In examples, the slidable couplers 362 may have forms other than a ring. For example, slide rails, magnetic couplings, or other forms of slidable couplers may be utilized as desired.

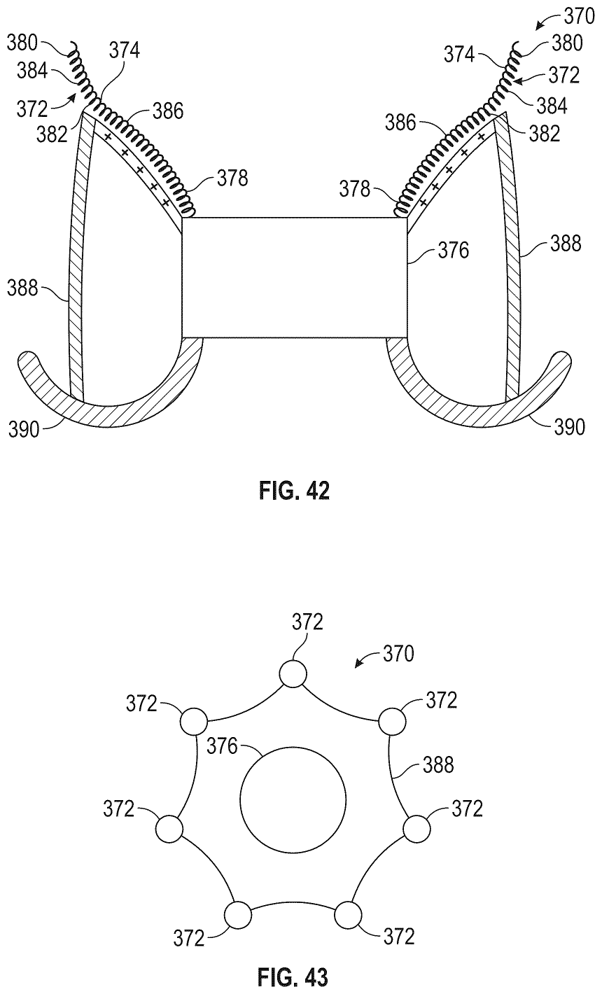

FIG. 42 illustrates a prosthetic valve 370 including one or more anchors 372 each including a coil 374. The coil 374 may form an elongate arm extending radially outward from a valve frame 376 of the prosthetic valve 370.

Each coil 374 may include a first or inner portion 378 coupled to the valve frame 376 and a second or outer portion 380 extending radially outward from the inner portion 378. Each coil 374 may extend from a proximal portion of the valve body comprising the valve frame 376. The coil 374 may comprise a flexible elongate arm configured to deflect to conform to a shape of a native valve. Referring to the top schematic view shown in FIG. 43, the anchors 372 may be circumferentially spaced from each other and may each extend radially outward from the valve frame 376.

In examples, the coils 374 may have equal flexibility along the length of the coil 374 or may include one or more portions that may have a varied flexibility. The coils 374 for example, may have a portion 382 that may have enhanced flexibility relative to a radially outward portion 384 of the coil 374 and relative to a radially inward portion 386 of the coil 374. The coils 374 accordingly may be configured to deflect about the portion 382 inward and/or outward as desired.

In examples, each anchor 372 may be configured similarly as the anchors 120 discussed with respect to FIGS. 11 and 12. For example, the anchor 372 may include one or more coil portions, a stiffener arm, and/or a cover that may be similar as the anchors 120 discussed with respect to FIGS. 11 and 12. In examples, the anchors 372 may comprise solely the coil 374.

The anchors 372 may comprise a proximal tensioning body that may be coupled to the sealing skirt 388 and may tension the sealing skirt 388. For example, the portion 382 of the coils 374 may be coupled to a proximal portion of the sealing skirt 388 and may support the sealing skirt 388 for sealing with a native valve. The sealing skirt 388 may be positioned radially outward from the valve frame 376 with a space between the sealing skirt 388 and the valve frame 376, the sealing skirt 388 being in tension and configured to deflect to conform to a shape of a native valve. A distal end of the sealing skirt 388 may couple to a distal anchor 390 or another portion of the prosthetic valve 370 as desired.

FIG. 43 illustrates a top schematic view of the prosthetic valve 370 showing one exemplary arrangement of anchors 372 that are spaced around the circumference.

The anchors 372 may comprise proximal or atrial anchors that may anchor the prosthetic valve 370 in the atrium of the heart. FIG. 44, for example, illustrates the prosthetic valve 370 deployed to the native valve 80 and anchored to the native valve leaflets 82. The anchors 372 may extend proximally and may contact the walls of the atrium or a portion of the native valve on the atrial side of the valve. The anchors 372 may be flexible to reduce the possibility of damage or a conduction disturbance for the native valve.

In examples, the distal anchors 390 may anchor the prosthetic valve 370 to the native valve in a similar manner as the distal anchors 17 discussed with respect to FIG. 1. The distal anchors 390 may comprise hooked anchors positioned at a distal portion of the valve body. The sealing skirt 388 may seal against the native valve to reduce flow outside of the flow channel of the valve 370.

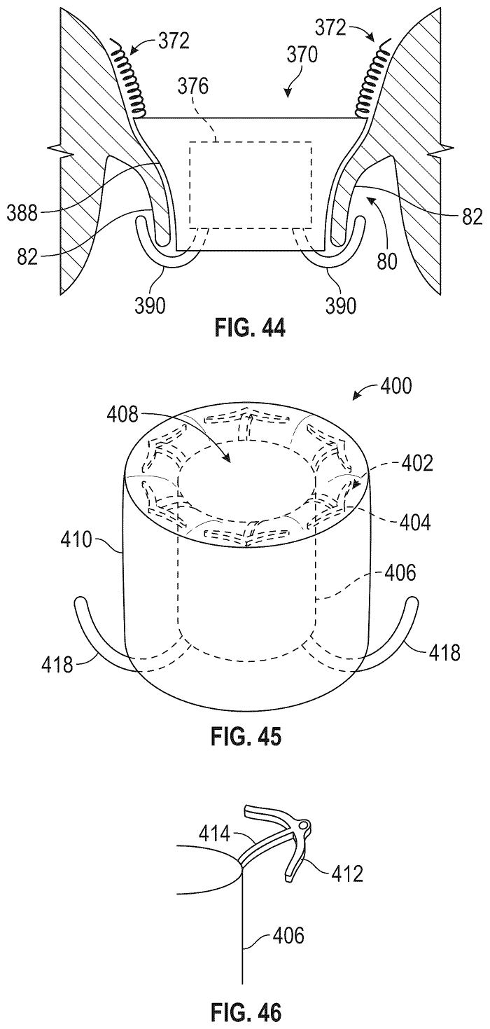

FIG. 45 illustrates an example of a prosthetic valve 400 including one or more anchors 402 configured to anchor prosthetic valve leaflets to a native valve, the one or more anchors 402 comprising a generally T-bar 404 shaped structure.

The prosthetic valve 400 may include a valve frame 406 surrounding a flow channel 408. One or more prosthetic valve leaflets may be positioned within a flow channel 408 and may be supported by the valve frame 406. A sealing skirt 410 may be positioned radially outward of the valve frame 406 and may be supported by the anchors 402. The sealing skirt 410, for example, may have a proximal portion and a distal portion, and the anchors 402 may comprise a proximal tensioning body coupled to the proximal portion of the sealing skirt 410 and configured to tension the sealing skirt 410.

Referring to FIG. 46, each T-bar 404 may comprise a cross bar 412 and a stem 414. The stem 414 may extend the cross bar 412 radially outward from the valve frame 406. Each stem 414 may be coupled to the proximal portion of the valve frame 406. The cross bar 412 may be positioned radially outward of the stem 414. The cross bar 412 may extend transverse to the stem 414. The cross bar 412 may comprise a support that may support the sealing skirt 410 in tension.

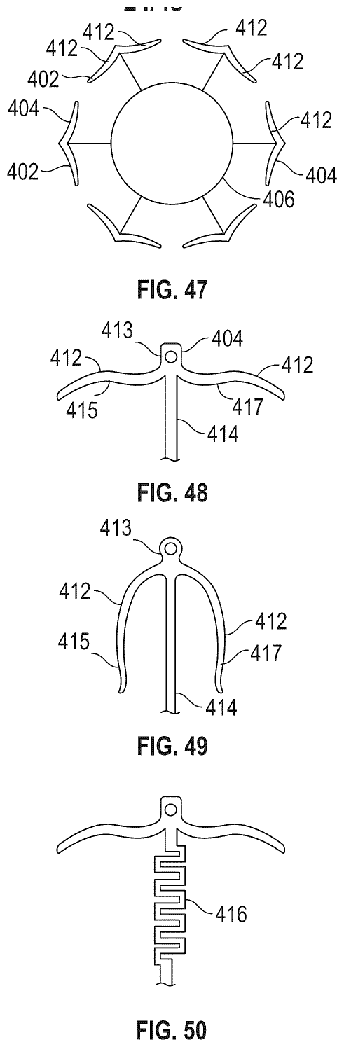

Referring to FIG. 47, each T-bar 404 may be spaced circumferentially from each other about the valve frame 406. The cross bars 412 of the plurality of anchors 402 may form a ring extending circumferentially about the prosthetic valve. A segmented flange extending circumferentially about the prosthetic valve may be formed by the cross bars 412 of the plurality of anchors 402. Each T-bar 404 may be positioned at a proximal portion of the valve frame 406 and may extend radially outward from the proximal portion of the valve frame.

In examples, each T-bar 404 may be configured to deflect radially inward. FIG. 48, for example, illustrates a top view of a T-bar 404 in which the arms of the cross bar 412 extend circumferentially outward with respect to the stem 414. The cross bar 412 may include a central portion 413 coupled to the stem 414 and a first arm 415 and a second arm 417 each extending outward from the central portion 413. The first arm 415 and the second arm 417 may extend outward circumferentially. In examples, the arms 415, 417 of the cross bar 412 may be deflected inward towards the stem 414, as shown in FIG. 49, for example.

The stem 414 may comprise a linearly extending stem 414, or in examples, may include an undulation. FIG. 50, for example illustrates a variation of a stem 416 including an undulation configured to provide flexibility for the stem 416. The undulation may increase the flexibility of the stem 416. The flexibility of the T-bar accordingly may be increased.

The anchors 402 may comprise proximal or atrial anchors that may support the prosthetic valve 400 within a native valve. The anchors 402 may be configured to be flexible to allow the anchors 402 to conform to a shape of a native valve.

In examples, the prosthetic valve 400 may include one or more distal anchors 418. The distal anchors 418 may be positioned at a distal portion of the valve frame 406. The distal anchors 418 may be configured similarly as the distal anchors described with respect to FIG. 1. The distal anchors 418, for example, may be configured to hook over a distal tip of a native valve leaflet to anchor the distal anchor 418 to the native valve. In examples, the distal anchors 418 may be coupled to a distal portion of the sealing skirt 410 and may tension the sealing skirt 410.

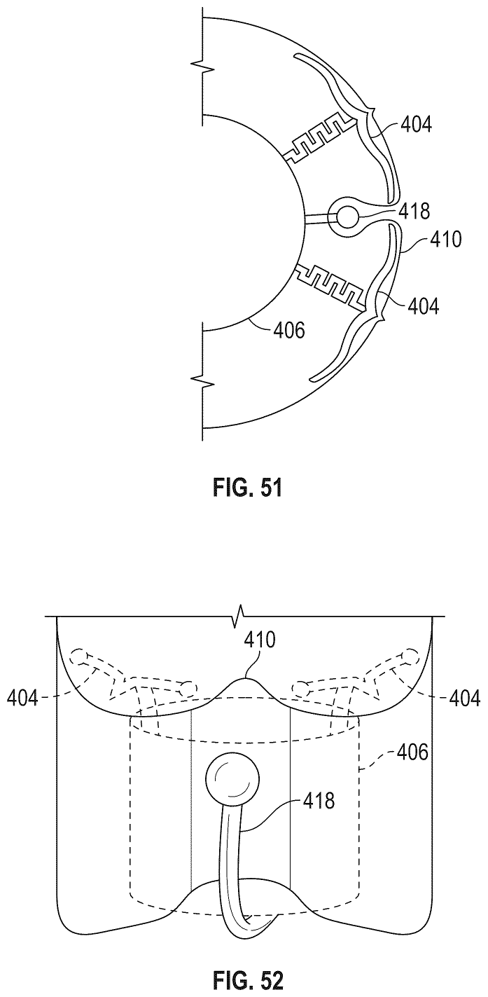

In examples, the distal anchors 418 may be positioned circumferentially between two of the proximal anchors comprising the adjacent T-bars 404. FIG. 51, for example, illustrates such a configuration of a distal anchor 418 and a T-bar 404. The sealing skirt 410 may extend between the distal anchors 418 and the valve frame 406 and may extend outward of the circumferentially adjacent T-bars 404. As such, upon missed capture of a leaflet by the distal anchor 418, the anchor may be positioned inward of the outer extent of the T-bars 404. The sealing skirt 410 covering the T-bars 404 may thus provide a seal at the portion of the native valve having the missed capture of the native leaflets.

FIG. 52, for example, illustrates a side perspective view showing the distal anchors 418 being positioned between the circumferentially adjacent T-bars 404.

The prosthetic valves disclosed with respect to FIGS. 27-52 may comprise single frame prosthetic valves or may have another configuration as desired. The features of the examples of FIGS. 27-52 may be utilized solely or in combination with any other example disclosed herein.

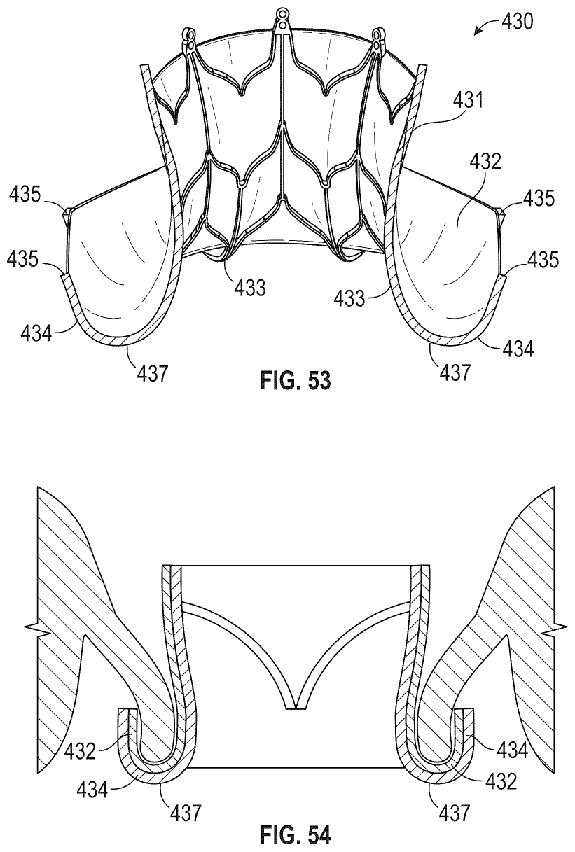

FIG. 53 illustrates an example of a prosthetic valve 430 in which a sealing skirt 432 forms a disk-shaped structure extending along the length of each of the elongate anchor arms 434. The disk-shaped structure may be continuous around the circumference and may extend between adjacent anchor arms 434.

The prosthetic valve 430 may include a valve body 431 supporting one or more prosthetic valve leaflets (as shown in FIG. 2 for example). The valve body 431 may comprise a frame as shown in FIG. 53 or may have another configuration as desired. A plurality of distal anchors may be provided in the form of the anchor arms 434.

The anchor arms 434 may comprise elongate arms each having a first end 433 coupled to the valve body 431 and extending radially outward along a length from the first end 433 to a tip 435 of the respective elongate arm. Each elongate anchor arm 434 may be configured to hook over a distal tip of a native valve leaflet to anchor the one or more prosthetic valve leaflets to the native valve. As such, the sealing skirt 432 may extend along the anchor arms 434 and may be positioned radially outward of the native valve leaflets. FIG. 54, for example, illustrates the position of the sealing skirt 432 radially outward of the native valve leaflets.

In examples, the sealing skirt 432 may be coextensive with each of the elongate anchor arms 434. The sealing skirt 432 as shown in FIGS. 53 and 54, may be closely mated along the elongate anchor arms 434 and extend along the length of the arms 434. Each elongate anchor arm 434 may have a curved portion 437 and the disk formed by the sealing skirt 432 may extend along the curved portion 437.

The radially outward extent of the sealing skirt 432 may be varied. In examples, the sealing skirt 432 may extend from the first end 433 of the anchor arms 434 to the tip 435 of each anchor arm 434. In examples, a lesser extent may be provided (e.g., along only a portion of the anchor arm 434). The sealing skirt 432 may yet extend along the anchor arms 434 such that the sealing skirt 432 is positioned radially outward of the native valve leaflets.

The sealing skirt 432 may comprise a disk having a convex curvature as shown in FIG. 53. The sealing skirt 432 may extend circumferentially about the entirety of the valve body 431 and may form a continuous disk about the valve body 431.

In examples, the sealing skirt 432 may be coextensive with a frame of the valve body 431. For example, as shown in FIG. 53, the sealing skirt 432 may extend along the frame proximally to a proximal portion and proximal end of the frame. The sealing skirt 432 may continue along the frame distally and may be continuous with the portion of the sealing skirt 432 extending along the anchor arms 434. The prosthetic valve may comprise a single frame prosthetic valve in examples.

Improved sealing may result based on the sealing skirt 432 extending along the anchor arms 434 and forming the disk configuration. A smaller crimp profile may also be provided.

The frame may have a shape including a shoulder or an hourglass shape to reduce the possibility of the prosthetic valve migrating into a ventricle. The hourglass shape is also advantageous because the prosthetic leaflets conform to the inner wall of the hourglass, thereby reducing any gap between the prosthetic leaflets and the frame when the prosthetic leaflets are in an open configuration for allowing blood to pass through the prosthetic valve 430. The elimination of the gap reduces the amount of stagnant blood and thereby reduces the possibility of thrombus formation.

In examples, chordae of a ventricle may be severed to allow the disk to extend around the native valve leaflets. The chordae may be severed prior to deployment of the prosthetic valve to reduce the possibility of interference between the chordae and the sealing skirt 432. In examples, the sealing skirt 432 may include one or more slits to allow for deployment of the prosthetic valve without the chordae being severed. The chordae may be positioned within one or more of the slits upon deployment in examples.

The features of the examples of FIGS. 53 and 54 may be utilized solely or in combination with any other example disclosed herein.

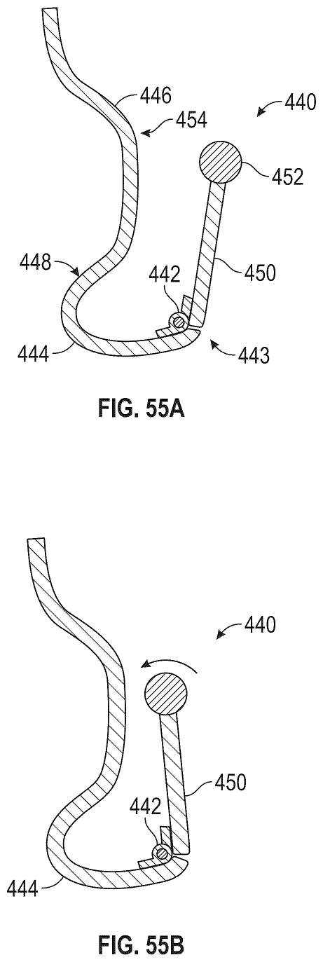

FIG. 55A illustrates a cross sectional view of an anchor 440 that may be utilized in examples herein. The anchor 440 may include a hinge 442 that may be configured to allow the anchor 440 to deflect radially.

The anchor 440 may be configured to be utilized with any example of prosthetic valve disclosed herein or with another form of prosthetic valve. The anchor 440 may include an inward portion or coupling portion 444 that may couple to a valve body 446. The valve body 446 may be configured similarly as any example of valve body disclosed herein, or may have another configuration in examples. The valve body 446 may support one or more prosthetic valve leaflets. The anchor 440 may couple to a distal end portion 448 of the valve body 446 in examples, or may be located at another position in examples as desired.

The anchor 440 may comprise an elongate arm in examples. The coupling portion 444 of the anchor 440 may be curved in examples, and may have a convex curvature in a distal direction. The coupling portion 444 may extend distally. The coupling portion 444 may be coupled to a tip portion 450 of the anchor 440. The coupling portion 444 may extend the tip portion 450 of the anchor 440 radially outward, to increase a radially outward position of the tip portion 450. In examples, the coupling portion 444 may have other configurations as desired.

The tip portion 450 of the anchor 440 may extend proximally upon deployment of the anchor 440. The tip portion 450 may extend linearly in examples from a juncture 443 with the coupling portion 444. The tip portion 450 may extend linearly in a proximal direction to a tip 452 of the anchor 440. The tip 452 may be axially aligned with a mid portion 454 of the valve body 446, or may be at another position in examples as desired.

The hinge 442 may be positioned at the juncture 443 of the tip portion 450 with the coupling portion 444 or may be at another position as desired. In examples, the hinge 442 may be positioned proximally on the tip portion 450 and closer to the tip 452 than shown in FIG. 55A. A portion of the anchor 440 positioned proximal or outward of the hinge 442 may comprise a pivoting portion of the anchor 440 and the portion of the anchor 440 positioned distal or inward of the hinge may comprise a static portion of the anchor 440. In examples, the hinge 442 may be positioned on the coupling portion 444.

The hinge 442 may be configured to allow the pivoting portion of the anchor 440 to deflect radially inward as represented in FIG. 55B. The hinge 442 may increase the flexibility of the anchor 440 in the direction radially inward. The hinge 442 may be configured to pivot about an axis extending transverse to a longitudinal axis of the prosthetic valve.

In examples, the hinge 442 may be configured to impede a radially outward deflection of the anchor 440. The hinge 442, for example, may comprise a uni-direction or single direction hinge 442 that may allow for radially inward movement and impede radially outward movement. In examples, a hinge may allow for radially outward deflection of the anchor 440.

The anchor 440 may be configured to be in a hooked configuration in examples. The anchor 440 may be configured to extend over a distal end or distal tip of a native valve leaflet for anchoring. The tip portion 450 may be positioned radially outward of a native valve leaflet in the hooked configuration.

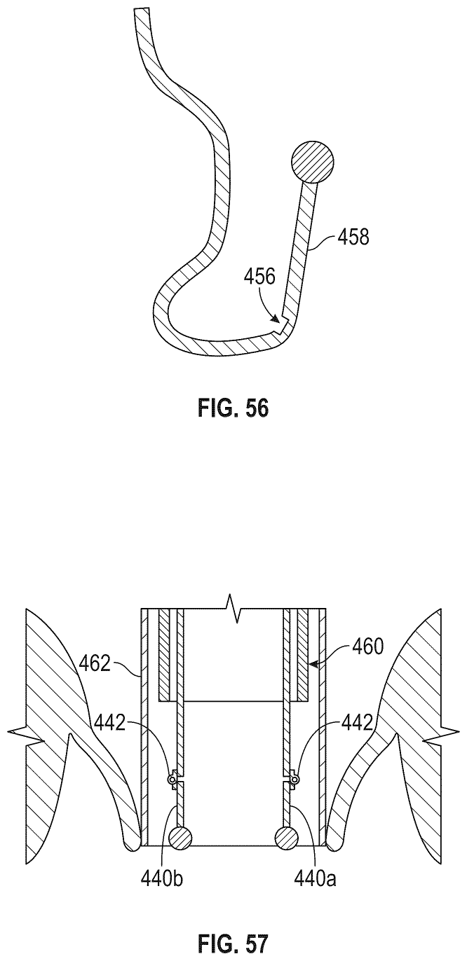

The hinge 442 may comprise a joint or link in examples. The hinge 442 may comprise a mechanical bearing hinge as shown in FIGS. 55A and 55B for example. The mechanical bearing hinge may include a plurality of components configured to rotate or otherwise move relative to each other to allow the hinge to operate. The mechanical bearing hinge may include an axle that the mechanical bearing hinge pivots about. In examples, the mechanical bearing hinge may be formed by a connector such as sutures or fabric joining the tip portion 450 to the coupling portion 444. A suture loop may serve as an axle in examples. In examples, a hinge may comprise a living hinge 456 as represented in FIG. 56. The living hinge 456 may comprise a thinner or weaker portion of the material comprising the anchor 458 that the anchor 458 may deflect about. The thinner or weaker portion may be on radially facing surface of the anchor 458 (such as a radially inward facing surface as shown in FIG. 56), or may be on one or more side surfaces (e.g, circumferentially facing surfaces) of the anchor 458.

In examples, the hinges may be spring biased to a radially outward position. The spring bias may be overcome with a radially inward force in examples. The spring bias may be produced by a portion of the hinge comprising a spring in examples. The materials comprising the hinge (e.g., a shape memory material, or other form of material) may comprise a spring bias material that may be biased to a radially outward position.

The hinges disclosed herein may allow the anchor to pivot to allow for greater compliance of the anchor to an implantation site upon implantation. For example, the anchor may be configured to deflect radially inward to accommodate a shape of a native heart valve. Reduced radial pressure against an implantation site may result. A reduced possibility of electrical conduction disturbance to the native anatomy may result. For example, reduced force against a conduction structure of a heart (e.g., a AV node) or heart valve annulus may result.

In examples, the hinges disclosed herein may be configured to allow the anchor to capture or recapture a native valve leaflet that the anchor has missed capture of. The capture or recapture may occur during a deployment sequence of a prosthetic valve in examples. In examples, the capture or recapture may occur following a deployment sequence.

The capture or recapture of a native valve leaflet may occur in response to a movement of the native valve leaflets. The movement may comprise a systolic movement of the native valve leaflets in examples. The systolic movement may be utilized in a deployment to a mitral or tricuspid valve in examples. The hinge may be configured to allow the respective anchor to deflect radially inward in response to a radially inward force of the native valve leaflet.

For example, FIGS. 57-62 illustrate a deployment sequence of a prosthetic valve 460 utilizing one or more of the hinges 442. A prosthetic valve utilizing one of the living hinges 456 may operate in a similar manner.

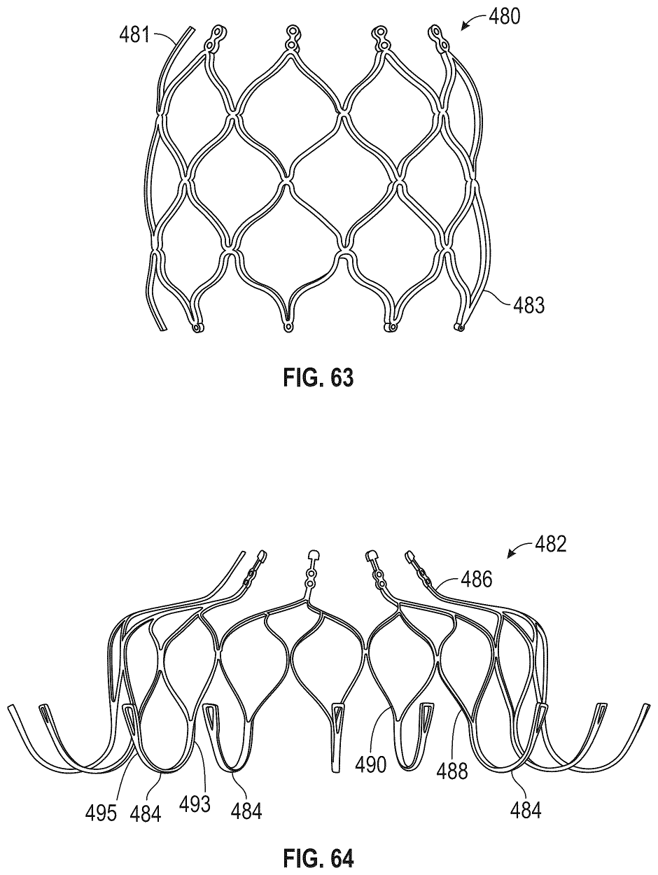

Referring to FIG. 62, the prosthetic valve 460 may be configured similarly as any example of prosthetic valve disclosed herein or any other form of prosthetic valve. The prosthetic valve 460 may include features similar to the prosthetic valve discussed in regard to FIGS. 1-8 for example. The prosthetic valve 460 may include a valve body 446, which may include an inner valve body 447 or inner frame in examples. The valve body 446 may include a proximal end portion 449 and a distal end portion 448, and the anchors 440 may be coupled to the distal end portion 448 of the valve body 446. One or more anchors 440 may be utilized (a plurality of anchors 440a, b are shown in FIG. 62). The anchors 440 may extend radially outward from the valve body 446. The anchors 440 may be circumferentially spaced from each other.

The valve body 446 may include a sealing body 466 positioned radially outward of the inner valve body 447. The sealing body 466 may include a distal end portion 453 and a proximal end portion 455. A proximal end portion 457 of the inner valve body 447 may be coupled to the proximal end portion 455 of the sealing body 466. The anchors 440 may be coupled to a distal end portion 459 of the inner valve body 447. The inner valve body 447 may include an inner frame and the sealing body 466 may include an outer frame. In examples, a sealing skirt may be positioned upon the outer frame.

The distal end portion 459 of the inner valve body 447 may be spaced from the distal end portion 453 of the sealing body 466 with a gap 461.

Referring to FIG. 57, the prosthetic valve 460 is shown in a compressed or crimped configuration within a capsule 462 of a delivery apparatus. In the configuration shown in FIG. 57, the anchors 440a, b, which each may be configured similarly as the anchor 440, are shown in an elongated or extended configuration within the capsule 462. The prosthetic valve 460 may be in a compressed or crimped configuration for approach to the implantation site.

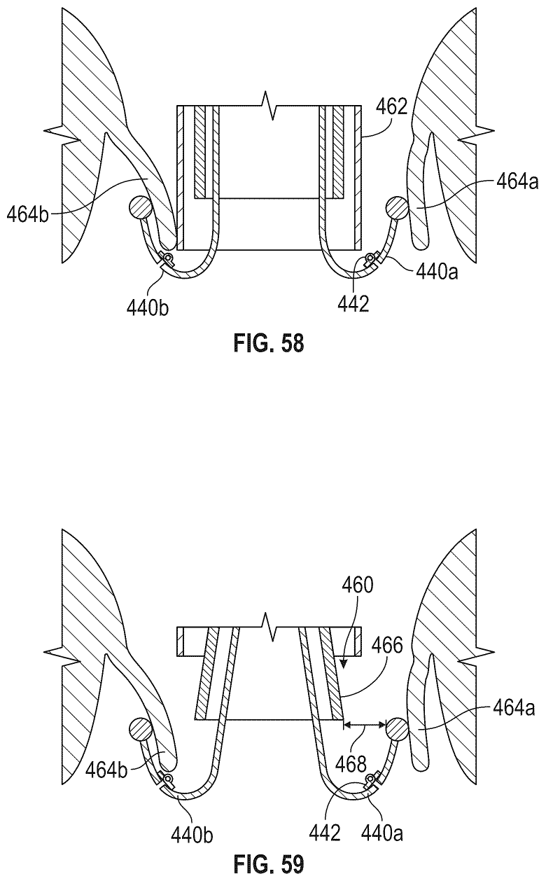

The anchors 440a, b may be released from the capsule 462 in a manner disclosed herein. For example, referring to FIG. 58, the capsule 462 may be retracted and the anchors 440a, b may be allowed to expand radially outward. The anchor 440b may capture and be positioned radially outward of the native valve leaflet 464b. The anchor 440a, however, may miss capture or fail to capture the native valve leaflet 464a and may be positioned radially inward of the native valve leaflet 464a. The hinge 442 on the anchor 440a may be utilized to allow for capture or recapture of the native valve leaflet 464a. In examples, the systolic movement of the leaflet 464a may cause the hinge 442 to pivot to allow for capture or recapture of the native valve leaflet 464a.

The movement of the anchor 440a may occur due to the radially inward movement of the leaflet 464a under a systolic closing movement of the leaflet 464a. The hinge 442 may pivot inward to allow the anchor 440a to be positioned radially outward of the leaflet 464a. The leaflet 464a may pass over the tip of the anchor 440a in a radially inward direction to be positioned radially inward of the anchor 440a. The hinge 442 may pivot to accommodate the movement of the leaflet 464a.

FIGS. 59-61, for example, illustrate such a movement of the leaflet 464a. Referring to FIG. 59, the leaflet 464a is shown positioned radially outward of the anchor 440a. The prosthetic valve 460 may be in a process of being deployed, and may be partially retained in a compressed configuration radially inward. The compression of the prosthetic valve 460, and particularly the outer frame or sealing body 466 of the prosthetic valve 460 may increase the gap clearance 468 between the anchor 440a and the body of the prosthetic valve 460, which may increase the ability of the anchor 440a to rotate radially inward. The space for the tip of the anchor 440a to rotate may be increased.

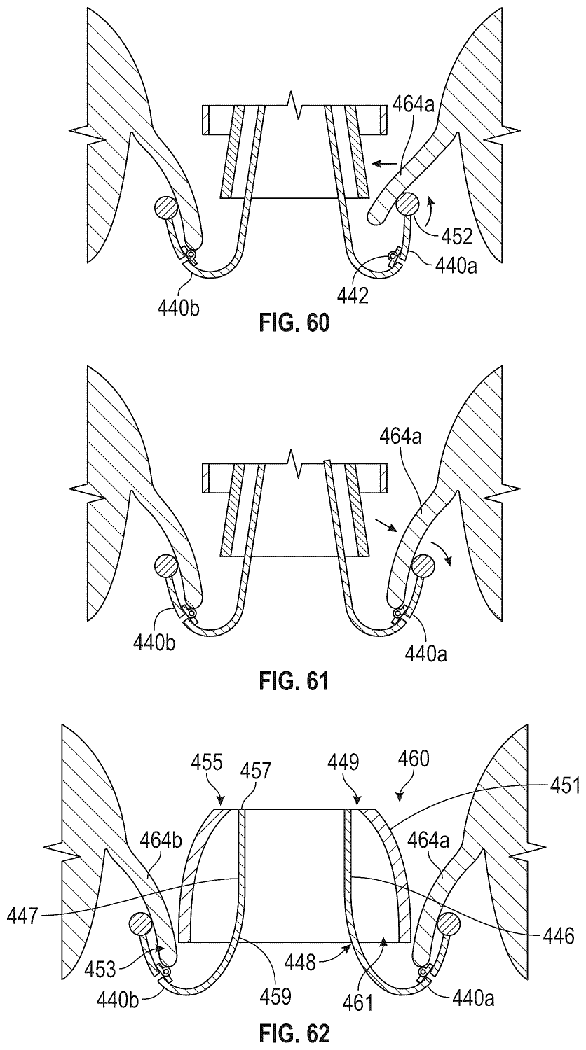

Upon deployment the leaflets 464a, b may move according to diastolic and systolic movement. The leaflet 464b may be constrained from radially outward movement during diastole by the anchor 440b. The leaflet 464b, however, may be capable of radially inward movement during systole. The leaflet 464a may move radially inward during systole. The inward movement of the leaflet 464a may cause the hinge 442 on the anchor 440a to pivot radially inward. The leaflet 464a may move radially inward of the anchor 440a as represented in FIG. 60 for example. The leaflet 464a may move radially inward past the tip 452 of the anchor 440a in examples.

The configuration of the hinge 442 on the anchor 440a may impede radially outward movement of the leaflet 464a during diastole, and the leaflet 464a accordingly may be captured by the anchor 440a as represented in FIG. 61.

The prosthetic valve 460 may be fully deployed as represented in FIG. 62. The anchors 440a, b may capture the respective leaflets 464a, b.

In examples, the anchors 440a, b may be configured to deflect radially outward. The hinges utilized, for example, may be configured to allow for radially outward movement. The radially outward movement may increase the flexibility and compliance of the anchors 440a, b upon implantation. Other configurations may be utilized in examples.

In examples, the anchors disclosed herein may comprise a visual indicator under medical imaging of whether a leaflet is captured. A radially inward movement of the anchor may comprise an indication that leaflet capture has not occurred. The medical imaging may comprise ultrasound (e.g., echocardiography) or fluoroscopy, combinations of ultrasound and fluoroscopy, or other forms of medical imaging. The anchor may include a fluoroscopic marker in examples. The anchors may be visualized to determine whether capture of a leaflet or missed capture of a leaflet has occurred.

The features of the examples of FIGS. 55A-62 may be utilized solely or in combination with any other example disclosed herein.

FIGS. 63-67 illustrate an example of a frame that may be utilized in examples herein. The frame may include an inner frame 480 as represented in FIG. 63 for example. The inner frame 480 may be configured to support one or more prosthetic valve leaflets, similar to other examples of inner frames disclosed herein. The inner frame 480 may include a proximal end portion 481 and a distal end portion 483. The frame may include an outer frame 482 as represented in FIG. 64 for example.

The outer frame 482 may include one or more anchors 484 that may be configured to anchor the frame to an implantation site. The one or more anchors 484 may be coupled to the outer frame 482 and may be configured to be positioned radially outward of one or more leaflets of a native valve to anchor to the native valve. The anchors 484 may comprise anchors having a hooked configuration or may comprise other forms of anchors in examples.

The outer frame 482 may include a proximal end portion 486 and a distal end portion 488. The anchors 484 may be positioned at the distal end portion 488 of the outer frame 482 in examples. The outer frame 482 may include a plurality of struts having a lattice structure. The struts of the outer frame 482 may form a plurality of distal apices 490 in examples. The anchors 484 may extend distally from the apices 490. Other positions may be utilized in examples.

Each anchor 484 may include a coupling portion 493 and a tip portion 495. The coupling portion 493 may be coupled to the outer frame 482 and the tip portion 495 may be configured to extend proximally from the coupling portion 493. The coupling portion 493 may couple to the distal end portion 488 of the outer frame 482. The coupling portion 493 may couple to a respective one of the distal apices 490 in examples. The coupling portion 493 may have a convex curvature in a distal direction (as shown in FIG. 64), or may have another configuration in examples as desired. The tip portion 495 may extend linearly from the coupling portion 493 in examples.

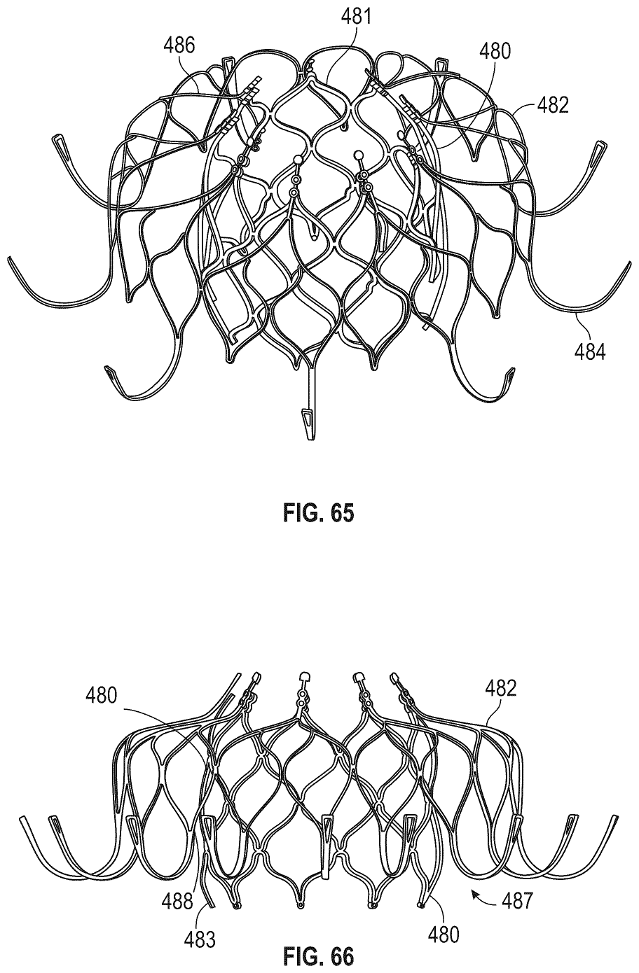

FIG. 65 illustrates a perspective view of the inner frame 480 coupled to the outer frame 482. The proximal end portion 481 of the inner frame 480 may couple to the proximal end portion 486 of the outer frame 482.

FIG. 66 illustrates a side view of the inner frame 480 coupled to the outer frame 482. The outer frame 482 may be positioned radially outward from the inner frame 480 and may surround the inner frame 480. The distal end portion 483 of the inner frame 480 may be separated from the distal end portion 488 of the outer frame 482 with a gap 487 in examples.

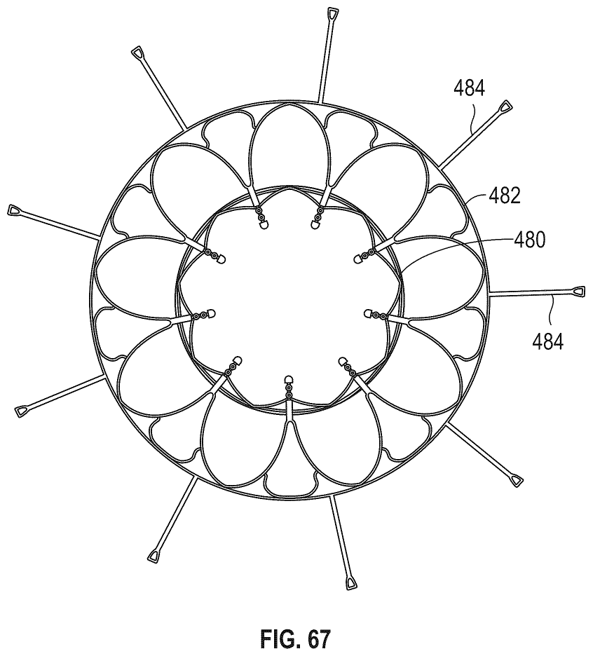

FIG. 67 illustrates a top view of the inner frame 480 coupled to the outer frame 482. The anchors 484 are shown to protrude radially outward from the outer frame 482. The anchors 484 may be circumferentially spaced from each other.

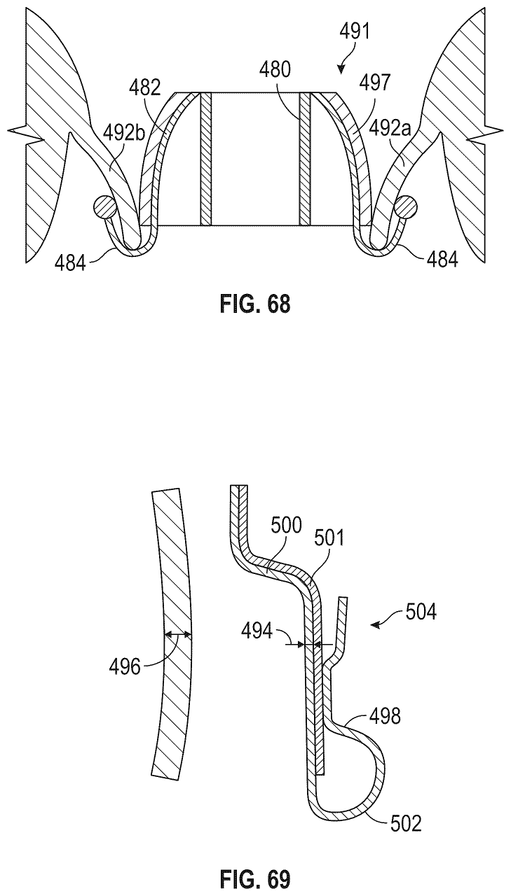

Upon deployment, the anchors 484 may be utilized to extend over native valve leaflets and be positioned radially outward of the native valve leaflets for anchoring in examples. FIG. 68, for example, illustrates a prosthetic valve 491 having been deployed to a native valve with the anchors 484 positioned radially outward of the native valve leaflets 492a, b. A sealing skirt 497 may be positioned upon the outer frame 482 as disclosed herein in examples.

The prosthetic valve 491 may include one or more prosthetic valve leaflets, similar to other examples of prosthetic valves disclosed herein. The prosthetic valve 491 may be configured similarly as examples of prosthetic valves disclosed herein unless stated otherwise.

The outer frame 482 may beneficially include the anchors 484 such that the inner frame 480 would receive pressure loads (directed to the movement of the prosthetic valve leaflets), while anchoring loads would be received by the outer frame 482. As such, the inner frame 480 would have less load upon it than in a configuration in which the inner frame includes anchors (as represented in FIG. 3 for example). The inner frame may further have reduced crimp strain upon it in a crimping operation.

In examples, the outer frame may have a lesser thickness 494 (marked in FIG. 69) in a radial dimension (a radial thickness) than a thickness 496 of the inner frame (marked in FIG. 69) in the radial dimension. The anchors 484 accordingly may have a lesser thickness 494 than the thickness 496 of the inner frame. The lesser thickness 494 of the outer frame and anchors may allow the anchors to be shaped into tighter or smaller radii of curvature than with a thicker frame. For example, anchors having a same thickness as the outer frame may be able to be shaped into tighter or smaller radii of curvature than anchors having a same thickness as the inner frame. Thinner anchors may further exert lower forces upon an implantation site. Reduced force against an annulus of a heart for example may result. A reduced possibility of electrical conduction disturbance may result.

In examples, an anchor 498 may be configured to clamp with a portion of a prosthetic valve. The anchor 498 may be configured to clamp a leaflet between the anchor 498 and a portion of the prosthetic valve to anchor to the leaflet. FIG. 69, for example, illustrates a configuration in which the anchor 498 is configured to clamp a leaflet between the anchor 498 and the outer frame 500 to secure a leaflet between the anchor 498 and the outer frame 500. The anchor 498 may provide a radially inward force against the leaflet to press the leaflet against the outer frame 500.

The anchor 498 may be biased radially inward against the outer frame 500. The anchor 498, for example, may include a distal loop portion 502 that may angle the tip portion 504 of the anchor 498 proximally and direct the tip portion 504 radially inward towards the outer frame 500. The tip portion 504 may comprise a clamping portion configured to clamp against the outer frame 500. A sealing skirt 501 may be coupled to the outer frame 500 in examples.

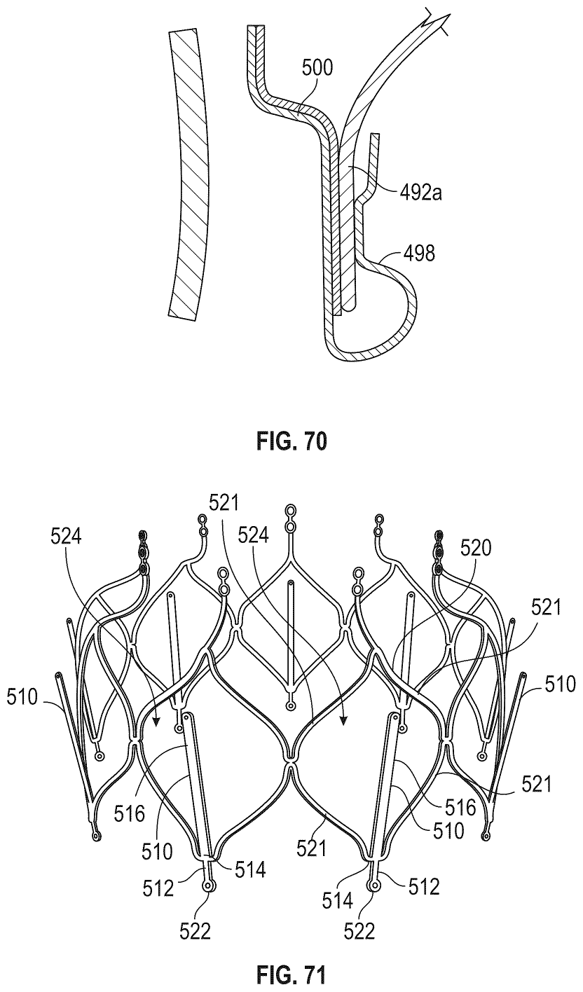

The anchors 498 may be held in an elongated configuration prior to deployment (similar to a configuration as shown in FIG. 57). The tip portions 504 may extend distally. The anchors 498 may be released to extend radially outward and flip in position to extend proximally (as represented in FIG. 70). The anchors 498 may be positioned radially outward of the leaflet 492a and may rotate to clamp toward the outer frame 500. The anchors 498 may press the native valve leaflet 492a radially inward and against the outer frame 500 as represented in FIG. 70. The native valve leaflet 492a may be clamped in position and such clamping may resist proximal (or atrial) and distal (or ventricular) movement of the prosthetic valve. Improved anchoring to the implantation site may result.

The features of the examples of FIGS. 63-70 may be utilized solely or in combination with any other example disclosed herein.

FIGS. 71-76 illustrates an example in which anchors 510 extend proximally. The anchors 510 may comprise elongate arms that may extend linearly in examples. The anchors 510 may each comprise a lever arm that may include a counter lever portion 512 on an opposite side of a fulcrum 514. The tip portion 516 of the anchor 510 may extend proximally from the fulcrum 514. The anchor 510 may be configured such that a radially inward force applied to the counter lever portion 512 may cause the tip portion 516 of the anchor 510 to pivot radially outward about the fulcrum 514 and extend the distance of the tip portion 516 from the outer frame 520. The anchor 510 may be biased to have the tip portion 516 move radially inward when the radially inward force applied to the counter lever portion 512 is reduced. The anchors 510 may be configured to be actuated to vary a radial position of the anchors 510. The anchors 510 may be coupled to the frame 520 via sutures or another form of coupling in examples. The anchors 510 may be coupled to distal apices of the frame 520 or to another portion as desired.

The counter lever portion 512 may include one or more actuator couplers 522 that may be configured to couple with an actuator assembly in examples. The actuator assembly may comprise a component of a delivery system in examples and may be utilized to actuate the anchors 510 during a deployment procedure as desired. The actuator couplers 522, for example, may comprise eyelets configured to engage with one or more tethers of an actuator assembly or may have another configuration as desired. The actuator couplers 522 may be positioned at a distal end portion of an outer frame 520 in examples. The actuator couplers 522 may be configured to be pulled radially inward by the actuator assembly to deflect the tip portions 516 of the anchors 510 radially outward.

The outer frame 520 may include a plurality of struts 521 forming a lattice structure. The lattice structure may have a plurality of openings 524. The tip portion 516 of the anchors 510 may be aligned with an opening 524 of the outer frame 520 that is bound by one or more struts in examples. The position of the anchors 510 may allow the anchors 510 to be drawn radially inward and reduce the outer diameter of the prosthetic valve in a compressed or crimped configuration.

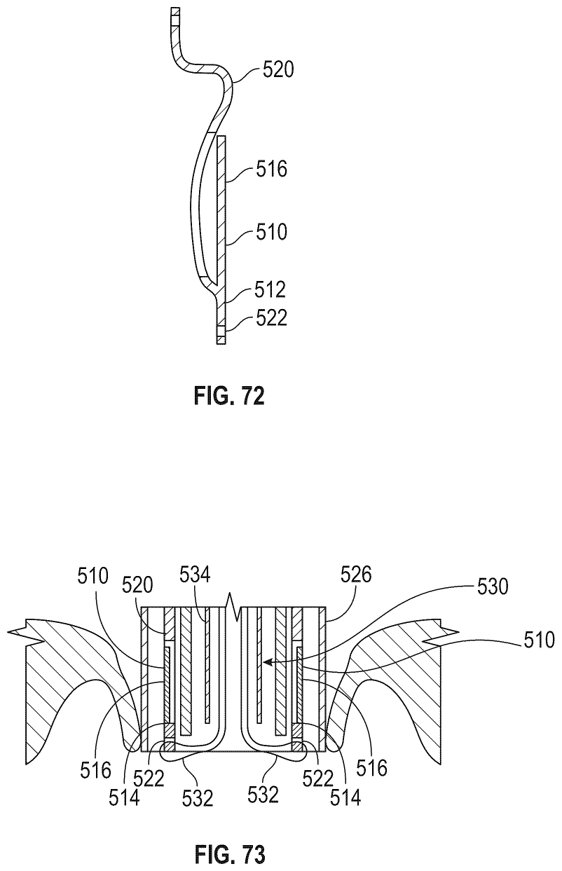

FIG. 72 illustrates a side cross sectional view illustrating a position of the anchors 510. The tip portion 516 of the anchors 510 may extend proximally.

FIGS. 73-76 illustrate an exemplary deployment sequence of a prosthetic valve utilizing the outer frame 520 and anchors 510. FIG. 73 illustrates the prosthetic valve in a compressed or crimped configuration within a capsule 526 of a delivery system. The anchors 510 may extend proximally with the tip portions 516 extending proximally from the fulcrums 514. The tip portion 516 of each of the anchors 510 is configured to extend proximally when the prosthetic valve is in a crimped configuration.

Each of the anchors 510 may be configured to be actuated to deflect the tip portion 516 radially outward from the outer frame 520.

In examples, the actuator assembly 530 may be utilized to actuate the anchors 510. The actuator assembly 530, for example, may include one or more tethers 532 configured to engage the couplers 522. The tethers 532 may comprise a cord, cable, wire, or other form of tether in examples. The tethers 532 may form a loop with two lengths extending through the delivery system. As such, a length of the tether 532 may be cut to allow the tether 532 to be pulled from a coupler 522 to release from the respective coupler 522. The actuator assembly 530 may include a shaft 534 that may engage the tethers 532. The shaft 534 may include a sheath or shroud that may extend over the tethers 532 and allow the tethers 532 to be pulled or released to actuate the anchors 510.

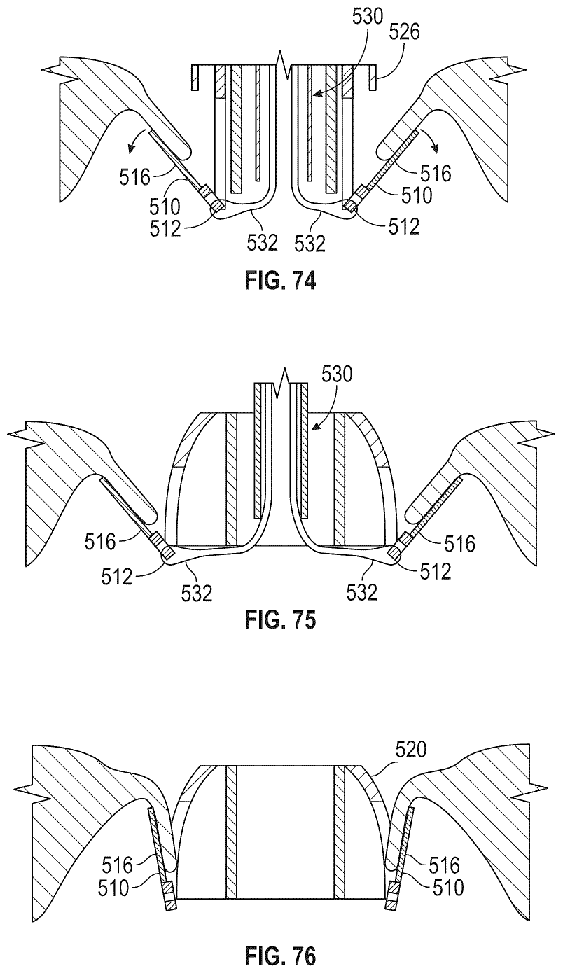

FIG. 74 illustrates the prosthetic valve having been partially released from the capsule 526. The actuator assembly 530 may be operated to apply a radially inward force to the counter lever portions 512 of the anchors 510. The tethers 532, for example, may be drawn radially inward to pull the counter lever portions 512 radially inward. The radially inward movement of the counter lever portions 512 may extend the tip portions 516 radially outward. The tip portions 516 may be positioned radially outward of the native valve leaflets, in a position to anchor to the leaflets. In examples, the prosthetic valve may be positioned distal of the native valve annulus, or moved distally to allow the tip portions 516 to hook over or be positioned radially outward of the native valve leaflets. The prosthetic valve may be retracted proximally in examples to seat the native valve leaflets upon the tip portions 516.

The prosthetic valve may continue to be released and expanded as represented in FIG. 75. The actuator assembly 530 may continue to apply a radially inward force to the counter lever portions 512. The tip portions 516 may be in an expanded state radially outward. The actuator assembly 530 may be released to allow the tip portions 516 to move radially inward as represented in FIG. 76. The anchors 510 may anchor the prosthetic valve in position. The anchors 510 may apply a clamping force to the native valve leaflets. The anchors 510 may clamp the native valve leaflets against the outer frame 520. The anchors 510 may press the native valve leaflets against a sealing skirt positioned upon the outer frame 520 and covering an opening 524 as marked in FIG. 71. The anchors 510 may apply a clamping force in another position as desired. The clamping force may resist a proximal (or atrial) movement of the prosthetic valve and a distal (or ventricular) movement of the prosthetic valve.

The features of the examples of FIGS. 71-76 may be utilized solely or in combination with any other example disclosed herein.

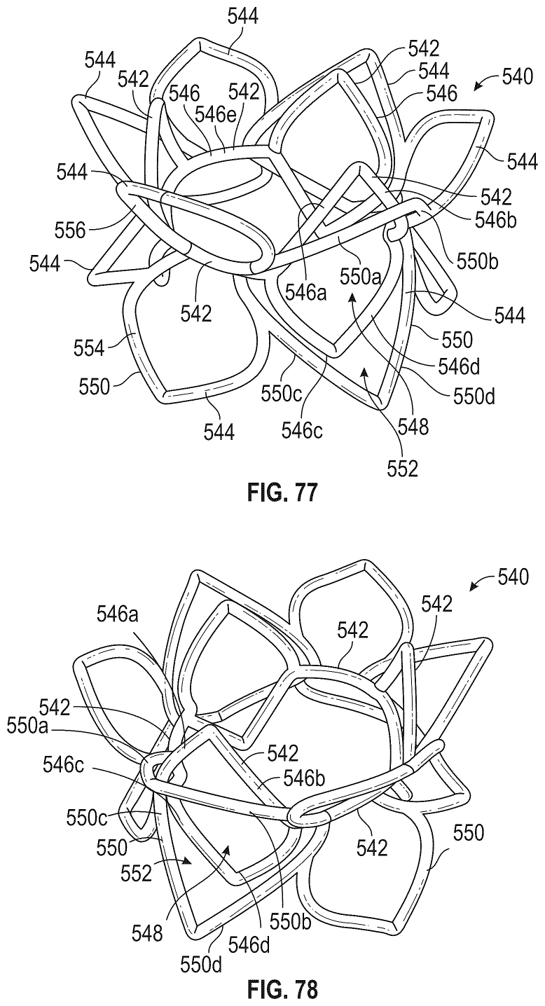

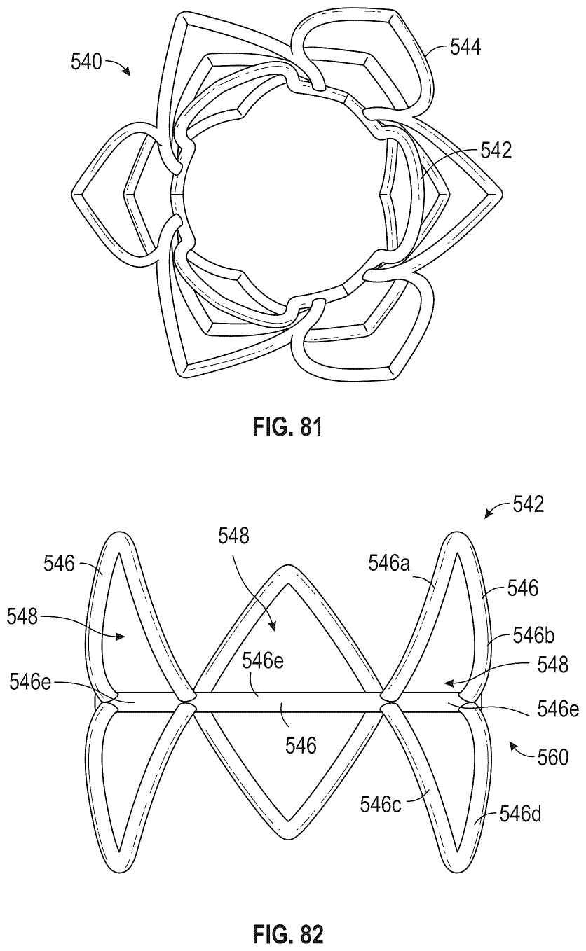

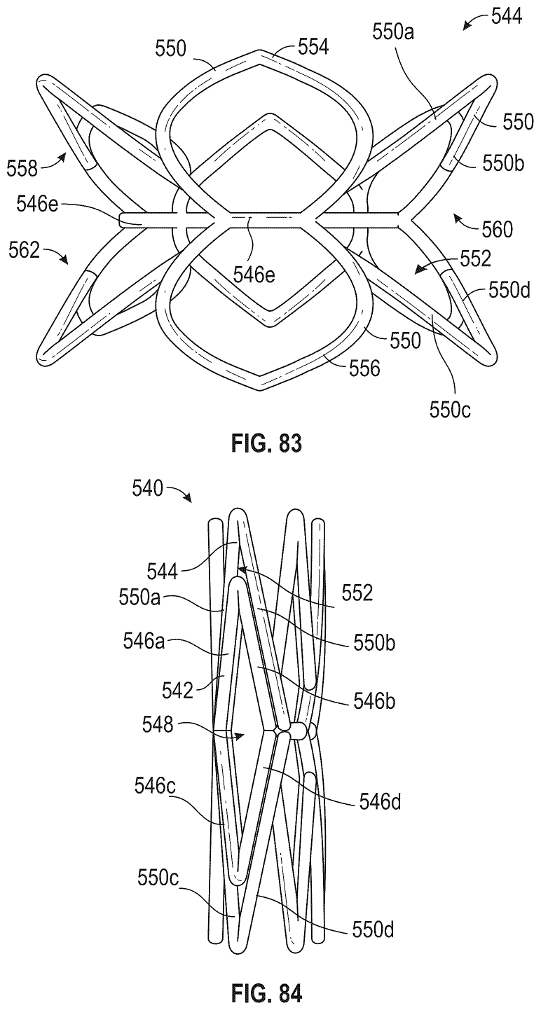

FIGS. 77-84 illustrate a frame 540 of a prosthetic valve that may be utilized in examples herein. The frame 540 is shown in an expanded or deployed configuration in FIG. 77. The frame 540 may include an inner frame 542 and may include an outer frame 544. The inner frame 542 is shown isolated from the outer frame 544 in FIG. 82. The outer frame 544 is shown isolated from the inner frame 542 in FIG. 83.

The portions of the frame 540 comprising the inner frame 542 and the outer frame 544 may be cut from a single piece of material in examples. As such, the outer frame 544 may be integral or unitary with the inner frame 542 in examples. Material having a cylindrical shape may be cut to form the portions of the frame 540 comprising the inner frame 542 and the outer frame 544. The outer frame 544 may be deflected radially outward from the inner frame 542. The outer frame 544, for example, may be deflected outward and shape set to be positioned outward of the inner frame 542 in examples.

The inner frame 542 may have a cylindrical shape in the expanded or deployed configuration as represented in FIG. 77. The inner frame 542 may comprise a plurality of struts 546. The plurality of struts 546 may bound a plurality of openings 548. The struts 546 may be formed into a lattice structure in which a plurality of struts 546a-d form a strut cell in examples. An exemplary strut cell is marked in FIG. 77 as comprising the four struts 546a-d forming a diamond shape and bounding an opening 548. The strut cell may comprise a closed strut cell in examples, which is fully bound by the struts 546a-d. Three of such strut cells are shown in FIG. 77 circumferentially spaced from each other. The inner frame in examples, may include circumferentially extending struts 546e that may join the strut cells to each other. Other configurations of an inner frame 542 may be utilized in examples.

The inner frame 542 may be configured to support one or more prosthetic valve leaflets in examples. FIG. 77 illustrates a configuration that may be utilized with three prosthetic valve leaflets. The commissures of the prosthetic valve leaflets may be positioned at each of the three strut cells of the inner frame 542 shown in FIG. 77.

The outer frame 544 may comprise a plurality of struts 550. The struts 550 may be formed into a lattice structure in which a plurality of struts 550a-d form a strut cell in examples. An exemplary strut cell is marked in FIG. 77 as comprising the plurality of struts 550a-d forming a diamond shape and bounding an opening 552. Three of such strut cells are shown in FIG. 77 circumferentially spaced from each other. The outer frame in examples, may include smaller strut cells 554, 556 each extending proximally and distally respectively from a central portion or mid portion of the outer frame 544. The smaller strut cells 554, 556 may be positioned circumferentially between the larger strut cells. Three pairs of the smaller strut cells 554, 556 are shown in FIG. 77 positioned circumferentially between adjacent larger strut cells.

The outer frame 544 and inner frame 542 may each comprise the circumferentially extending struts 546e.

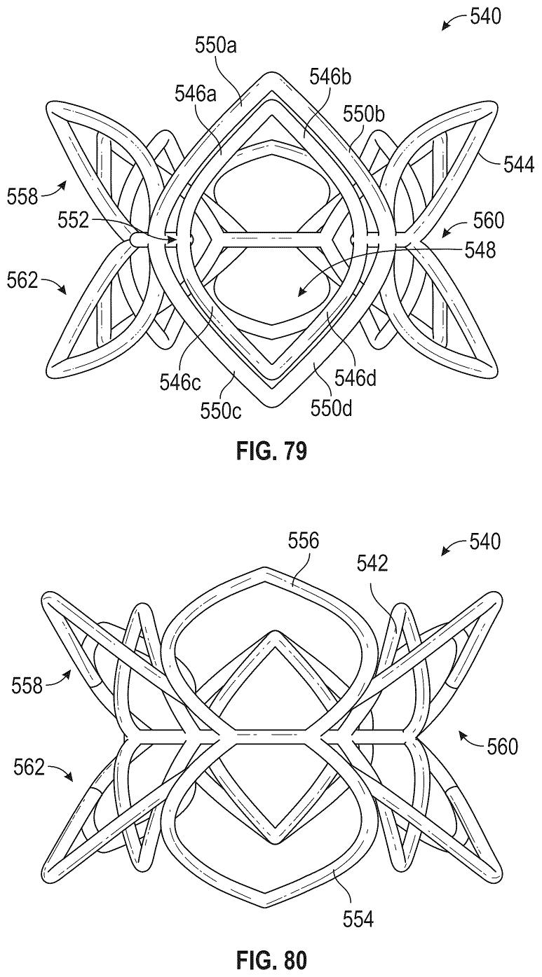

At least a portion of the inner frame 542 is aligned within at least one of the plurality of openings 552 of the outer frame 544. The strut cell formed by the struts 546a-d, for example, is aligned within the opening 552 of the outer frame 544 that is bound by the struts 550a-d. FIG. 79, for example, illustrates such alignment. The strut cell formed by the struts 546a-d may comprise a closed strut cell. The diamond shaped strut cell of the inner frame 542 may be aligned within the diamond shaped strut cell of the outer frame 544.

FIG. 78 illustrates a perspective view of the frame 540 rotated from the position shown in FIG. 77.

FIG. 79 illustrates a side view of the frame 540. The outer frame 544 may include a proximal portion 558, a distal portion 562, and a central portion 560. The outer frame 544 is shown to have an hourglass shape in the expanded configuration with the proximal portion 558 flaring radially outward from the central portion 560 of the outer frame 544. The distal portion 562 of the outer frame 544 flares radially outward from the central portion 560 of the outer frame 544. The central portion 560 may comprise a narrow portion or waist portion positioned between the proximal portion 558 and the distal portion 562.

FIG. 80 illustrates a side view of the frame 540 rotated from the position shown in FIG. 79. The inner frame 542 may have a cylindrical shape (more clearly shown in FIG. 82) in the expanded configuration. In examples, the inner frame 542 may have other configurations including a non-cylindrical or asymmetric profile.

FIG. 81 illustrates a top view of the frame 540. FIG. 82 illustrates an isolated view of the inner frame 542 without the outer frame 544 shown. FIG. 83 illustrates an isolated view of the outer frame 544 without the inner frame 542 shown.

The frame 540 may be configured to expand radially outward from a crimped or compressed configuration to an expanded configuration. The inner frame 542 and outer frame 544 may each have a cylindrical shape in the crimped or compressed configuration.

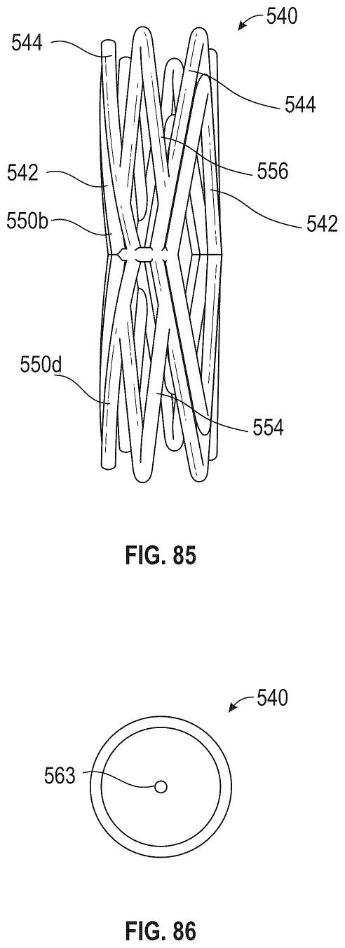

The configuration of the frame 540 being cut from a single piece of material may allow for the frame 540 to be moved to a crimped or compressed configuration in which strut cells of the inner frame 542 may be positioned within openings of strut cells of the outer frame 544. Such a configuration is shown in FIG. 84 for example. At least a portion of the inner frame 542 is within at least one of the openings of the outer frame 544 when the frame 540 is in the crimped or compressed configuration. The position of the inner frame 542 within the openings of the strut cells of the outer frame 544 may allow for a reduced outer diameter of the prosthetic valve due to the outer frame not being layered upon the inner frame as may result in a configuration shown in FIG. 5 for example. FIG. 85, for example, illustrates a view of the frame 540 in a crimped or compressed configuration at a view rotated from the view shown in FIG. 84.

FIG. 86 illustrates a top schematic view of the frame 540 in a crimped or compressed configuration. The outer frame 544 (marked in FIG. 85) is at the same diameter or radial distance as the inner frame 542 in such a configuration. The frame may surround a central axis 563 of the prosthetic valve.

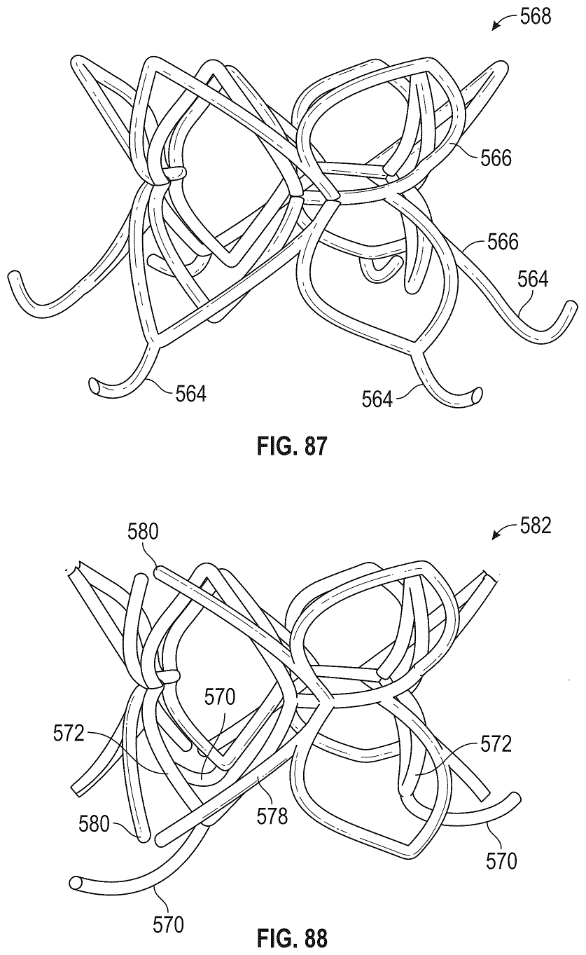

Variations in the frame 540 may be provided. FIG. 87, for example, illustrates a configuration in which one or more distal anchors 564 may be coupled to the outer frame 566. The distal anchor 564 may couple to distal apices of the struts of the outer frame 566 in examples. The distal anchors 564 may form hooks for anchoring to the native valve. The distal anchors 564 may have a hooked configuration. The frame 568 may otherwise be configured similarly as the frame 540. The distal anchors 564 may be configured to be positioned radially outward of native valve leaflets in examples.

FIG. 88 illustrates a variation in which distal anchors 570 may be coupled to the inner frame 572. The distal anchor 570 may couple to distal apices of the struts of the inner frame 572 in examples. The distal anchors 570 may form hooks for anchoring to the native valve. The distal anchors 570 may have a hooked configuration. Cells of the outer frame 578 may comprise open strut cells with cut outs 580 that may allow passage of the distal anchors 570 radially outward for anchoring. The cut outs 580 may further allow the frame 582 to have a uniform diameter upon crimping or compression (as represented in FIG. 86 for example). At least a portion of the inner frame 572 may be aligned with at least a portion of the plurality of open strut cells. The frame 582 may otherwise be configured similarly as the frame 540.

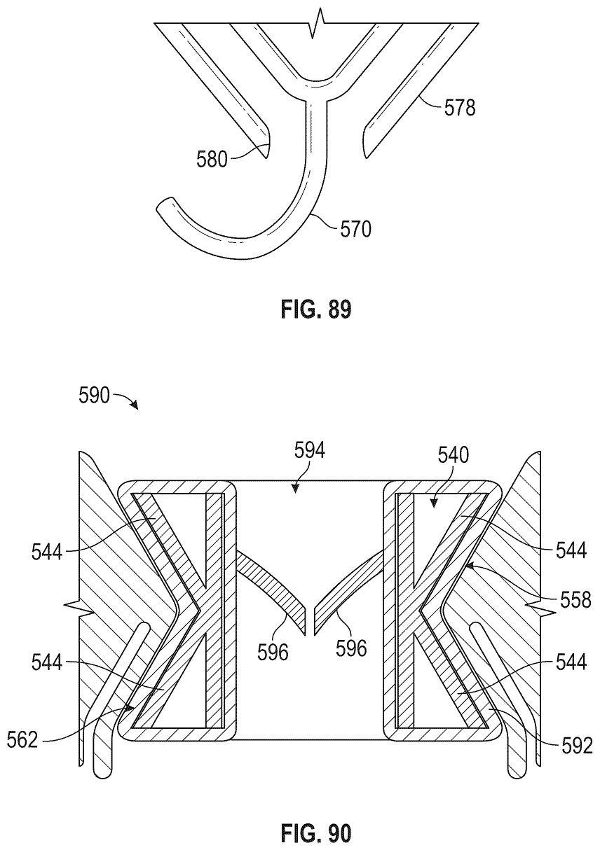

FIG. 89 illustrates a schematic view of the position of the cut outs 580 relative to the distal anchor 570 to allow for passage through the outer frame 578.

The anchors 564, 570 may extend distally and may comprise distal anchors of the respective frames.

In examples, the anchors 564, 570 may be configured to form hooks for clamping a leaflet of a native valve. The anchors 564, 570 may clamp the leaflet against a portion of the frame in a manner disclosed in regard to FIG. 70 or in another manner as desired. The anchors 564, 570 may clamp the leaflet to the respective outer frames in examples.

In examples, upon deployment, the outer frame 544 may comprise an anchoring body that may be utilized to anchor the prosthetic valve 590 to an implantation site. FIG. 90, for example, illustrates a deployed configuration of the prosthetic valve 590. The proximal portion 558 of the outer frame 544 may comprise a proximal anchor and the distal portion 562 of the outer frame 544 may comprise a distal anchor. The proximal anchor may be positioned on an atrial side of the native valve. The distal anchor may be positioned on a ventricular side of the native valve. A flared shape of the proximal portion 558 and the distal portion 562 may serve to impede ventricular and atrial movement respectively of the prosthetic valve 590.

In examples, a sealing skirt 592 may be positioned upon the frame 540. The sealing skirt 592 may extend over the openings of the struts to cover and seal fluid flow therethrough. The prosthetic valve 590 may include one or more prosthetic valve leaflets 596. In examples, an interior portion of the sealing skirt 592 may bound a flow channel 594 of the prosthetic valve 590 that the prosthetic valve leaflets 596 may be positioned within. The prosthetic valve leaflets 596 may be sutured or otherwise coupled to the sealing skirt 592.

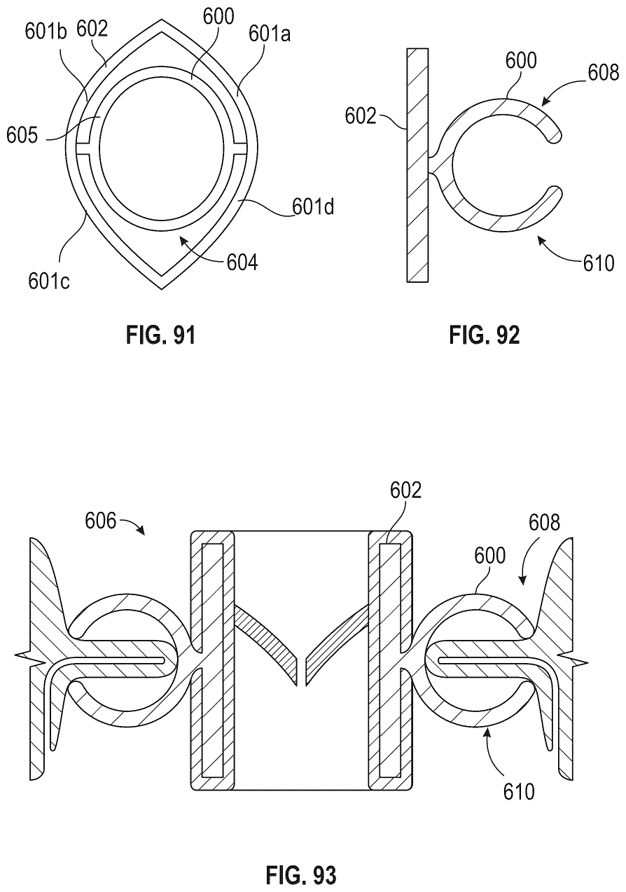

Variations in the frame 540 and prosthetic valve 590 may be provided. FIGS. 91-93, for example, illustrate a variation in which the struts forming the inner frame 542 in FIG. 77 are deflected radially outward to form an outer frame 600, and the struts forming the outer frame 544 in FIG. 77 are maintained in a cylindrical configuration to form an inner frame 602. As such, the cells of the outer frame 600 may be aligned with the openings 604 of the cells of the inner frame 602 in examples. In a compressed or crimped configuration, the frame may have a configuration shown in FIGS. 84 and 85 for example. At least a portion of the outer frame 600 is within at least one of the openings of the inner frame 602 when the frame is in the crimped configuration.

FIG. 91 illustrates a side view of a cell of the outer frame 600 aligned within an opening 604 of the inner frame 602. At least a portion of the outer frame 600 is aligned within at least one of the plurality of openings 604 of the inner frame 602. The strut cell formed by the struts 601a-d (corresponding to struts 550a-d shown in FIG. 77) of the inner frame 602 may comprise a closed strut cell. The diamond shaped strut cell 605 (corresponding to struts 546a-d shown in FIG. 77) of the outer frame 600 may be aligned within the diamond shaped strut cell of the inner frame 602.

FIG. 92 illustrates a side view of the outer frame 600 protruding from the inner frame 602.

In examples, the outer frame 600 may be configured to clamp with a portion of a heart. The outer frame 600, for example, may clamp with a native heart valve annulus to anchor the prosthetic valve in position. The outer frame 600 may clamp a portion of a heart between a proximal portion 608 and a distal portion 610 of a frame 600 to anchor the frame to the heart. FIG. 93 illustrates an exemplary deployed configuration, in which the outer frame 600 clamps to an atrial and ventricular side of a native heart valve to anchor the prosthetic valve 606 in position. Other configurations may be utilized in examples.

The features of the examples of FIGS. 55A-93 may be utilized solely or in combination with any other example disclosed herein.

The examples of prosthetic valves may be utilized in a mitral valve as disclosed herein or may be utilized in other deployment locations such as a native tricuspid valve, or other deployment locations. Deployment to aortic or pulmonary valves, as well as other implantation sites are contemplated.

Various modifications of the examples disclosed herein may be provided. Features of examples may be modified, substituted, excluded, or combined across examples as desired. Combinations of features across examples may be provided as desired. Combinations of features may be provided across examples with other features of such examples being excluded if desired.

The various examples of sealing skirts disclosed herein may have a variety of forms, including cloth skirts, foam skirts, or braided skirts as desired. Various materials may be utilized as desired.

The implants disclosed herein may include prosthetic heart valves or other forms of implants, such as stents or filters, or diagnostic devices, among others. The implants may be expandable implants configured to move from a compressed or undeployed state to an expanded or deployed state. The implants may be compressible implants configured to be compressed inward to have a reduced outer profile and to move the implant to the compressed or undeployed state.

Various forms of delivery apparatuses may be utilized with the examples disclosed herein. The delivery apparatuses as disclosed herein may be utilized for aortic, mitral, tricuspid, and pulmonary replacement and repair as well. The delivery apparatuses may comprise delivery apparatuses for delivery of other forms of implants, such as stents or filters, or diagnostic devices, among others.

The implants and the systems disclosed herein may be used in transcatheter mitral or tricuspid implantation, as well as aortic valve implantation (TAVI) or replacement of other native heart valves (e.g., pulmonary valves). The delivery apparatuses and the systems disclosed herein may be utilized for transarterial access, including transfemoral access, to a patient's heart. The delivery apparatuses and systems may be utilized in transcatheter percutaneous procedures, including transarterial procedures, which may be transfemoral or transjugular. Transapical procedures, among others, may also be utilized. Other procedures may be utilized as desired.

In addition, the methods herein are not limited to the methods specifically described, and may include methods of utilizing the systems and apparatuses disclosed herein. The steps of the methods may be modified, excluded, or added to, with systems, apparatuses, and methods disclosed herein. The examples disclosed herein may comprise systems for implantation within a human body in examples.

For purposes of this description, certain aspects, advantages, and novel features of the examples of this disclosure are described herein. The disclosed methods, apparatuses, and systems should not be construed as limiting in any way. Instead, the present disclosure is directed toward all novel and nonobvious features and aspects of the various disclosed examples, along and in various combinations and sub-combinations with one another. The methods, apparatuses, and systems are not limited to any specific aspect or feature or combination thereof, nor do the disclosed examples require that any one or more specific advantages be present or problems be solved. Features, elements, or combinations of one example can be combined into other examples herein.

Example 1: A prosthetic valve configured to be deployed to a native valve, the prosthetic valve comprising: one or more prosthetic valve leaflets; a sealing body positioned radially outward of the one or more prosthetic valve leaflets and configured to seal against a portion of the native valve; one or more anchors configured to anchor the one or more prosthetic valve leaflets to the native valve; and one or more magnets configured to draw the sealing body and at least one of the one or more anchors together.

Example 2: The prosthetic valve of any example herein, in particular example 1, wherein the sealing body includes a frame.

Example 3: The prosthetic valve of any example herein, in particular example 2, wherein the frame comprises an outer frame positioned radially outward of an inner frame, the inner frame supporting the one or more prosthetic valve leaflets.

Example 4: The prosthetic valve of any example herein, in particular example 3, wherein the outer frame includes a proximal portion coupled to the inner frame and a distal portion, and the distal portion includes the one or more magnets or one or more ferromagnetic materials configured to be attracted to the one or more magnets.

Example 5: The prosthetic valve of any example herein, in particular examples 1-4, wherein the sealing body includes a sealing skirt.

Example 6: The prosthetic valve of any example herein, in particular examples 1-5, wherein the one or more magnets are positioned on the one or more anchors.

Example 7: The prosthetic valve of any example herein, in particular examples 1-6, wherein the one or more magnets are positioned on the sealing body.

Example 8: The prosthetic valve of any example herein, in particular examples 1-7, further comprising one or more ferromagnetic materials, wherein the one or more magnets are configured to attract the one or more ferromagnetic materials.

Example 9: The prosthetic valve of any example herein, in particular example 8, wherein the one or more ferromagnetic materials are positioned on the sealing body and the one or more magnets are positioned on the one or more anchors; or the one or more ferromagnetic materials are positioned on the one or more anchors and the one or more magnets are positioned on the sealing body.

Example 10: The prosthetic valve of any example herein, in particular examples 1-9, wherein the one or more anchors are configured to be in a hooked configuration.

Example 11: The prosthetic valve of any example herein, in particular examples 1-10, wherein each of the one or more anchors is configured to hook over a distal tip of a native valve leaflet.

Example 12: The prosthetic valve of any example herein, in particular examples 1-11, wherein the one or more anchors are configured to be positioned radially outward of a native valve leaflet and the sealing body is configured to be positioned radially inward of the native valve leaflet.

Example 13: The prosthetic valve of any example herein, in particular example 12, wherein the one or more magnets are configured to clamp the native valve leaflet between the sealing body and the one or more anchors.

Example 14: The prosthetic valve of any example herein, in particular examples 1-13, wherein the one or more magnets comprise a plurality of magnets positioned on the sealing body and spaced circumferentially from each other.

Example 15: The prosthetic valve of any example herein, in particular examples 1-14, wherein the prosthetic valve is configured to be deployed to a mitral valve or a tricuspid valve.

Example 16: A method comprising: deploying a prosthetic valve to a native valve, the prosthetic valve including: one or more prosthetic valve leaflets, a sealing body positioned radially outward of the one or more prosthetic valve leaflets and configured to seal against a portion of the native valve, one or more anchors configured to anchor the one or more prosthetic valve leaflets to the native valve, and one or more magnets configured to draw the sealing body and at least one of the one or more anchors together.

Example 17: The method of any example herein, in particular example 16, wherein the sealing body includes a frame.

Example 18: The method of any example herein, in particular example 17, wherein the frame comprises an outer frame positioned radially outward of an inner frame, the inner frame supporting the one or more prosthetic valve leaflets.

Example 19: The method of any example herein, in particular example 18, wherein the outer frame includes a proximal portion coupled to the inner frame and a distal portion, and the distal portion includes the one or more magnets or one or more ferromagnetic materials configured to be attracted to the one or more magnets.

Example 20: The method of any example herein, in particular examples 16-19, wherein the sealing body includes a sealing skirt.

Example 21: The method of any example herein, in particular examples 16-20, wherein the one or more magnets are positioned on the one or more anchors.

Example 22: The method of any example herein, in particular examples 16-21, wherein the one or more magnets are positioned on the sealing body.

Example 23: The method of any example herein, in particular examples 16-22, wherein the one or more magnets are configured to attract one or more ferromagnetic materials.

Example 24: A prosthetic valve configured to be deployed to a native valve, the prosthetic valve comprising: one or more prosthetic valve leaflets; and one or more anchors configured to anchor the one or more prosthetic valve leaflets to the native valve, each of the one or more anchors including one or more grooves configured to increase a flexibility of the respective anchor.

Example 25: The prosthetic valve of any example herein, in particular example 24, wherein each of the one or more anchors is configured to be in a hooked configuration.

Example 26: The prosthetic valve of any example herein, in particular example 24 or example 25, wherein each of the one or more anchors is configured to hook over a distal tip of a native valve leaflet.

Example 27: The prosthetic valve of any example herein, in particular examples 24-26, wherein each of the one or more anchors extends radially outward from a valve body supporting the one or more prosthetic valve leaflets, and each of the one or more anchors includes a surface facing radially inward and a surface facing radially outward, and the one or more grooves are positioned on the surface facing radially inward.

Example 28: The prosthetic valve of any example herein, in particular example 27, wherein the one or more grooves are positioned on the surface facing radially outward.

Example 29: The prosthetic valve of any example herein, in particular examples 24-28, wherein each of the one or more anchors comprises an elongate arm having a length and the one or more grooves extend transverse to the length of the elongate arm.

Example 30: The prosthetic valve of any example herein, in particular example 29, wherein the one or more grooves comprise a plurality of grooves spaced from each other along the length of the elongate arm.

Example 31: The prosthetic valve of any example herein, in particular examples 24-30, wherein each of the one or more anchors includes a curved portion having a convex curvature, and the one or more grooves are positioned on the curved portion.

Example 32: The prosthetic valve of any example herein, in particular examples 24-31, wherein each of the one or more anchors extends radially outward from a valve body, and the one or more grooves increase a flexibility of the respective anchor in a direction radially inward.

Example 33: The prosthetic valve of any example herein, in particular examples 24-32, wherein the prosthetic valve is configured to be deployed to a mitral valve or a tricuspid valve.

Example 34: A method comprising: deploying a prosthetic valve to a native valve, the prosthetic valve including: one or more prosthetic valve leaflets, and one or more anchors configured to anchor the one or more prosthetic valve leaflets to the native valve, each of the one or more anchors including one or more grooves configured to increase a flexibility of the respective anchor.

Example 35: The method of any example herein, in particular example 34, wherein each of the one or more anchors is configured to be in a hooked configuration.

Example 36: The method of any example herein, in particular example 34 or example 35, wherein each of the one or more anchors is configured to hook over a distal tip of a native valve leaflet.

Example 37: The method of any example herein, in particular examples 34-36, wherein each of the one or more anchors extends radially outward from a valve body supporting the one or more prosthetic valve leaflets, and each of the one or more anchors includes a surface facing radially inward and a surface facing radially outward, and the one or more grooves are positioned on the surface facing radially inward.

Example 38: The method of any example herein, in particular example 37, wherein the one or more grooves are positioned on the surface facing radially outward.

Example 39: The method of any example herein, in particular examples 34-38, wherein each of the one or more anchors comprises an elongate arm having a length and the one or more grooves extend transverse to the length of the elongate arm.

Example 40: The method of any example herein, in particular example 39, wherein the one or more grooves comprise a plurality of grooves spaced from each other along the length of the elongate arm.

Example 41: The method of any example herein, in particular examples 34-40, wherein each of the one or more anchors includes a curved portion having a convex curvature, and the one or more grooves are positioned on the curved portion.

Example 42: A prosthetic valve configured to be deployed to a native valve, the prosthetic valve comprising: one or more prosthetic valve leaflets; and one or more anchors configured to anchor the one or more prosthetic valve leaflets to the native valve, each of the one or more anchors including a coil.

Example 43: The prosthetic valve of any example herein, in particular example 42, wherein the coil forms an elongate arm.

Example 44: The prosthetic valve of any example herein, in particular example 42 or example 43, wherein the coil includes a first coil portion and a second coil portion, the first coil portion having a greater stiffness than the second coil portion.

Example 45: The prosthetic valve of any example herein, in particular example 44, wherein each of the one or more anchors extends radially outward from a valve body supporting the one or more prosthetic valve leaflets, and the second coil portion is positioned radially outward of the first coil portion.

Example 46: The prosthetic valve of any example herein, in particular examples 42-45, wherein each of the one or more anchors includes a stiffener arm extending along the coil.

Example 47: The prosthetic valve of any example herein, in particular example 46, wherein each of the one or more anchors is curved to include an inner radius and an outer radius, and the stiffener arm extends along the outer radius outward of the coil.

Example 48: The prosthetic valve of any example herein, in particular examples 42-47, wherein each of the one or more anchors is configured to be in a hooked configuration.

Example 49: The prosthetic valve of any example herein, in particular examples 42-48, wherein each of the one or more anchors is configured to hook over a distal tip of a native valve leaflet.

Example 50: The prosthetic valve of any example herein, in particular examples 42-49, wherein each of the one or more anchors includes a curved portion having a convex curvature, and the coil is positioned on the curved portion.

Example 51: The prosthetic valve of any example herein, in particular examples 42-50, wherein each of the one or more anchors extends radially outward from a valve body, and the coil increases a flexibility of the respective anchor in a direction radially inward.

Example 52: The prosthetic valve of any example herein, in particular examples 42-51, wherein the coil comprises a spring coil.

Example 53: The prosthetic valve of any example herein, in particular examples 42-52, wherein the coil comprises a cut hypotube.

Example 54: The prosthetic valve of any example herein, in particular examples 42-53, wherein a valve body supports the one or more prosthetic valve leaflets and each of the one or more anchors includes a coupling portion for coupling to the valve body and extends radially outward to a tip portion of the respective anchor, and the coil extends from the coupling portion radially outward to the tip portion.

Example 55: The prosthetic valve of any example herein, in particular examples 42-54, wherein each of the one or more anchors includes an overmold positioned over the respective coil.

Example 56: The prosthetic valve of any example herein, in particular examples 42-55, wherein a valve body supports the one or more prosthetic valve leaflets and has a proximal portion and a distal portion, and the one or more anchors extend from the proximal portion of the valve body.

Example 57: The prosthetic valve of any example herein, in particular example 56, further comprising a plurality of hooked anchors positioned at the distal portion of the valve body.

Example 58: The prosthetic valve of any example herein, in particular examples 42-57, wherein the one or more anchors each comprise an atrial anchor configured to anchor within a heart atrium.

Example 59: The prosthetic valve of any example herein, in particular examples 42-58, wherein a valve body supports the one or more prosthetic valve leaflets, and the one or more anchors comprise a plurality of the anchors extending from the valve body and spaced circumferentially from each other.

Example 60: The prosthetic valve of any example herein, in particular examples 42-59, wherein the one or more anchors support a sealing body for sealing with the native valve.

Example 61: The prosthetic valve of any example herein, in particular examples 42-60, wherein the prosthetic valve is configured to be deployed to a mitral valve or a tricuspid valve.

Example 62: A method comprising: deploying a prosthetic valve to a native valve, the prosthetic valve including: one or more prosthetic valve leaflets, and one or more anchors configured to anchor the one or more prosthetic valve leaflets to the native valve, each of the one or more anchors including a coil.

Example 63: The method of any example herein, in particular example 62, wherein the coil forms an elongate arm.

Example 64: The method of any example herein, in particular example 62 or example 63, wherein the coil includes a first coil portion and a second coil portion, the first coil portion having a greater stiffness than the second coil portion.

Example 65: The method of any example herein, in particular example 64, wherein each of the one or more anchors extends radially outward from a valve body supporting the one or more prosthetic valve leaflets, and the second coil portion is positioned radially outward of the first coil portion.

Example 66: The method of any example herein, in particular examples 62-65, wherein each of the one or more anchors includes a stiffener arm extending along the coil.

Example 67: The method of any example herein, in particular example 66, wherein each of the one or more anchors is curved to include an inner radius and an outer radius, and the stiffener arm extends along the outer radius outward of the coil.

Example 68: The method of any example herein, in particular examples 62-67, wherein each of the one or more anchors is configured to be in a hooked configuration.

Example 69: The method of any example herein, in particular examples 62-68, wherein each of the one or more anchors is configured to hook over a distal tip of a native valve leaflet.

Example 70: A prosthetic valve configured to be deployed to a native valve, the prosthetic valve comprising: a valve body surrounding a flow channel extending along a longitudinal dimension; one or more prosthetic valve leaflets extending radially inward from the valve body and positioned within the flow channel; and one or more anchors pivotally coupled to the valve body and configured to pivot about the longitudinal dimension to vary a radial distance of the one or more anchors from the valve body.

Example 71: The prosthetic valve of any example herein, in particular example 70, wherein the flow channel extends along a central axis and each of the one or more anchors is configured to pivot about an axis extending parallel with the central axis.

Example 72: The prosthetic valve of any example herein, in particular example 70 or example 71, wherein each of the one or more anchors is pivotally coupled to the valve body with a hinge.

Example 73: The prosthetic valve of any example herein, in particular example 72, wherein each of the one or more anchors includes a tip configured to rotate about the respective hinge.

Example 74: The prosthetic valve of any example herein, in particular examples 70-73, wherein each of the one or more anchors is configured to pivot about the longitudinal dimension to vary a greatest radial extent of the prosthetic valve.

Example 75: The prosthetic valve of any example herein, in particular examples 70-74, wherein each of the one or more anchors has a length and the length is configured to remain constant upon the respective anchor pivoting about the longitudinal dimension.

Example 76: The prosthetic valve of any example herein, in particular examples 70-75, wherein each of the one or more anchors is configured to be in a hooked configuration.

Example 77: The prosthetic valve of any example herein, in particular examples 70-76, wherein each of the one or more anchors is configured to hook over a distal tip of a native valve leaflet.

Example 78: The prosthetic valve of any example herein, in particular examples 70-77, wherein each of the one or more anchors comprises a distal anchor and the prosthetic valve further comprises a proximal frame support coupled to the respective distal anchor and configured to pivot about the longitudinal dimension with the distal anchor.

Example 79: The prosthetic valve of any example herein, in particular examples 70-78, wherein the one or more anchors comprise a plurality of anchors pivotally coupled to the valve body and each configured to pivot about the longitudinal dimension to vary a radial distance of the plurality of anchors from the valve body.

Example 80: The prosthetic valve of any example herein, in particular example 79, wherein the plurality of anchors are circumferentially spaced from each other about the valve body.

Example 81: The prosthetic valve of any example herein, in particular examples 70-80, wherein each of the one or more anchors is configured to extend perpendicular with an outer surface of the valve body and is configured to extend parallel with the outer surface of the valve body.

Example 82: The prosthetic valve of any example herein, in particular examples 70-81, further comprising one or more locks configured to lock movement of the one or more anchors about the longitudinal dimension.

Example 83: The prosthetic valve of any example herein, in particular examples 70-82, wherein the valve body includes an outer surface comprising a sealing body, and the one or more anchors are configured to press a native valve leaflet to the sealing body.

Example 84: The prosthetic valve of any example herein, in particular examples 70-83, wherein the prosthetic valve is configured to be deployed to a mitral valve or a tricuspid valve.

Example 85: A method comprising: deploying a prosthetic valve to a native valve, the prosthetic valve including: a valve body surrounding a flow channel extending along a longitudinal dimension, one or more prosthetic valve leaflets extending radially inward from the valve body and positioned within the flow channel, and one or more anchors pivotally coupled to the valve body and configured to pivot about the longitudinal dimension to vary a radial distance of the one or more anchors from the valve body.

Example 86: The method of any example herein, in particular example 85, wherein the flow channel extends along a central axis and each of the one or more anchors is configured to pivot about an axis extending parallel with the central axis.

Example 87: The method of any example herein, in particular example 85 or example 86, wherein each of the one or more anchors is pivotally coupled to the valve body with a hinge.

Example 88: The method of any example herein, in particular example 87, wherein each of the one or more anchors includes a tip configured to rotate about the respective hinge.

Example 89: The method of any example herein, in particular examples 85-88, wherein each of the one or more anchors is configured to pivot about the longitudinal dimension to vary a greatest radial extent of the prosthetic valve.

Example 90: The method of any example herein, in particular examples 85-89, wherein each of the one or more anchors has a length and the length is configured to remain constant upon the respective anchor pivoting about the longitudinal dimension.

Example 91: The method of any example herein, in particular examples 85-90, wherein each of the one or more anchors is configured to be in a hooked configuration.

Example 92: The method of any example herein, in particular examples 85-91, wherein each of the one or more anchors is configured to hook over a distal tip of a native valve leaflet.

Example 93: A prosthetic valve configured to be deployed to a native valve, the prosthetic valve comprising: one or more prosthetic valve leaflets; a sealing body positioned radially outward of the one or more prosthetic valve leaflets and configured to seal against a portion of the native valve; one or more anchors configured to anchor the one or more prosthetic valve leaflets to the native valve; and a band configured to have an adjustable diameter to move the sealing body radially inward or outward.

Example 94: The prosthetic valve of any example herein, in particular example 93, further comprising an inner frame positioned radially inward of the sealing body.

Example 95: The prosthetic valve of any example herein, in particular example 93 or example 94, wherein the sealing body comprises a sealing skirt.

Example 96: The prosthetic valve of any example herein, in particular examples 93-95, wherein each of the one or more anchors is configured to hook over a distal tip of a native valve leaflet.

Example 97: The prosthetic valve of any example herein, in particular examples 93-96, wherein the band comprises a loop having an end and a portion configured to overlap the end.

Example 98: The prosthetic valve of any example herein, in particular example 97, further comprising a coupler positioned at the end, and the portion is configured to slide through the coupler to adjust the diameter of the band.

Example 99: The prosthetic valve of any example herein, in particular example 98, further comprising an adjustment mechanism positioned at the end and configured to adjust the diameter of the band.

Example 100: The prosthetic valve of any example herein, in particular examples 93-99, wherein the band includes a first end portion and a second end portion, the first end portion configured to overlap the second end portion.

Example 101: The prosthetic valve of any example herein, in particular example 100, wherein the first end portion is configured to extend along a length of the second end portion.

Example 102: The prosthetic valve of any example herein, in particular example 100 or example 101, wherein the band is configured to have the diameter adjusted by varying an amount of the first end portion that overlaps the second end portion.

Example 103: The prosthetic valve of any example herein, in particular examples 93-102, wherein the band is circular.

Example 104: The prosthetic valve of any example herein, in particular examples 93-103, wherein the band is configured to have the adjustable diameter adjusted in vivo.

Example 105: The prosthetic valve of any example herein, in particular examples 93-104, wherein the sealing body is elastic.

Example 106: The prosthetic valve of any example herein, in particular examples 93-105, wherein the band extends circumferentially about the sealing body.

Example 107: The prosthetic valve of any example herein, in particular examples 93-106, wherein the prosthetic valve is configured to be deployed to a mitral valve or a tricuspid valve.

Example 108: A method comprising: deploying a prosthetic valve to a native valve, the prosthetic valve including: one or more prosthetic valve leaflets, a sealing body positioned radially outward of the one or more prosthetic valve leaflets and configured to seal against a portion of the native valve, one or more anchors configured to anchor the one or more prosthetic valve leaflets to the native valve, and a band configured to have an adjustable diameter to move the sealing body radially inward or outward.

Example 109: The method of any example herein, in particular example 108, wherein an inner frame is positioned radially inward of the sealing body.

Example 110: The method of any example herein, in particular example 108 or example 109, wherein the sealing body comprises a sealing skirt.

Example 111: The method of any example herein, in particular examples 108-110, wherein each of the one or more anchors is configured to hook over a distal tip of a native valve leaflet.

Example 112: The method of any example herein, in particular examples 108-111, wherein the band comprises a loop having an end and a portion configured to overlap the end.

Example 113: The method of any example herein, in particular example 112, wherein a coupler is positioned at the end, and the portion is configured to slide through the coupler to adjust the diameter of the band.

Example 114: The method of any example herein, in particular example 113, wherein an adjustment mechanism is positioned at the end and is configured to adjust the diameter of the band.

Example 115: The method of any example herein, in particular examples 108-114, wherein the band includes a first end portion and a second end portion, the first end portion configured to overlap the second end portion.

Example 116: A prosthetic valve configured to be deployed to a native valve, the prosthetic valve comprising: a valve frame surrounding a flow channel; one or more prosthetic valve leaflets coupled to the valve frame and extending radially inward from the valve frame and positioned within the flow channel; and a sealing skirt positioned radially outward from the valve frame with a space between the sealing skirt and the valve frame, the sealing skirt being in tension and configured to deflect to conform to a shape of the native valve.

Example 117: The prosthetic valve of any example herein, in particular example 116, wherein the sealing skirt surrounds the valve frame.

Example 118: The prosthetic valve of any example herein, in particular example 116 or example 117, further comprising: a proximal tensioning body positioned at a proximal portion of the prosthetic valve; and a distal tensioning body positioned at a distal portion of the prosthetic valve, and wherein the sealing skirt includes a proximal end coupled to the proximal tensioning body and a distal end coupled to the distal tensioning body, the sealing skirt being tensioned by the proximal tensioning body and the distal tensioning body.

Example 119: The prosthetic valve of any example herein, in particular example 118, wherein the proximal tensioning body comprises an arm having a first end coupled to a proximal portion of the valve frame and a second end extending radially outward from the first end.

Example 120: The prosthetic valve of any example herein, in particular example 118 or example 119, wherein the distal tensioning body comprises an arm having a first end coupled to a distal portion of the valve frame and a second end extending radially outward from the first end.

Example 121: The prosthetic valve of any example herein, in particular examples 118-120, further comprising a plurality of the proximal tensioning bodies extending radially outward from a proximal portion of the valve frame and a plurality of the distal tensioning bodies extending radially outward from a distal portion of the valve frame.

Example 122: The prosthetic valve of any example herein, in particular example 121, wherein each of the plurality of the proximal tensioning bodies is spaced circumferentially equidistant from each other and each of the plurality of the distal tensioning bodies is spaced circumferentially equidistant from each other.

Example 123: The prosthetic valve of any example herein, in particular examples 118-122, wherein the distal tensioning body comprises a distal anchor for the prosthetic valve.

Example 124: The prosthetic valve of any example herein, in particular examples 118-123, wherein the proximal tensioning body comprises a proximal anchor for the prosthetic valve.

Example 125: The prosthetic valve of any example herein, in particular examples 118-124, wherein the proximal tensioning body comprises a ring.

Example 126: The prosthetic valve of any example herein, in particular examples 118-125, wherein the distal tensioning body comprises a ring.

Example 127: The prosthetic valve of any example herein, in particular examples 116-126, further comprising a plurality of wires extending along the sealing skirt and supporting the sealing skirt.

Example 128: The prosthetic valve of any example herein, in particular examples 116-127, wherein the sealing skirt is elastic and configured to deflect radially inward to conform to the shape of the native valve.

Example 129: The prosthetic valve of any example herein, in particular examples 116-128, wherein the prosthetic valve comprises a single frame prosthetic valve.

Example 130: The prosthetic valve of any example herein, in particular examples 116-129, wherein the prosthetic valve is configured to be deployed to a mitral valve or a tricuspid valve.

Example 131: A method comprising: deploying a prosthetic valve to a native valve, the prosthetic valve including: a valve frame surrounding a flow channel, one or more prosthetic valve leaflets coupled to the valve frame and extending radially inward from the valve frame and positioned within the flow channel, and a sealing skirt positioned radially outward from the valve frame with a space between the sealing skirt and the valve frame, the sealing skirt being in tension and configured to deflect to conform to a shape of the native valve.

Example 132: The method of any example herein, in particular example 131, wherein the sealing skirt surrounds the valve frame.

Example 133: The method of any example herein, in particular example 131 or example 132, wherein: a proximal tensioning body is positioned at a proximal portion of the prosthetic valve, a distal tensioning body is positioned at a distal portion of the prosthetic valve, and wherein the sealing skirt includes a proximal end coupled to the proximal tensioning body and a distal end coupled to the distal tensioning body, the sealing skirt being tensioned by the proximal tensioning body and the distal tensioning body.

Example 134: The method of any example herein, in particular example 133, wherein the proximal tensioning body comprises an arm having a first end coupled to a proximal portion of the valve frame and a second end extending radially outward from the first end.

Example 135: The method of any example herein, in particular example 133 or example 134, wherein the distal tensioning body comprises an arm having a first end coupled to a distal portion of the valve frame and a second end extending radially outward from the first end.

Example 136: The method of any example herein, in particular examples 133-135, wherein a plurality of the proximal tensioning bodies extends radially outward from a proximal portion of the valve frame and a plurality of the distal tensioning bodies extends radially outward from a distal portion of the valve frame.

Example 137: The method of any example herein, in particular example 136, wherein each of the plurality of the proximal tensioning bodies is spaced circumferentially equidistant from each other and each of the plurality of the distal tensioning bodies is spaced circumferentially equidistant from each other.

Example 138: The method of any example herein, in particular examples 133-137, wherein the distal tensioning body comprises a distal anchor for the prosthetic valve.

Example 139: A prosthetic valve configured to be deployed to a native valve, the prosthetic valve comprising: a valve frame surrounding a flow channel; one or more prosthetic valve leaflets coupled to the valve frame and extending radially inward from the valve frame and positioned within the flow channel; a sealing skirt positioned radially outward from the valve frame and including a pocket; and a ring positioned in the pocket and configured to support the sealing skirt.

Example 140: The prosthetic valve of any example herein, in particular example 139, wherein the pocket comprises overlapping material of the sealing skirt.

Example 141: The prosthetic valve of any example herein, in particular example 139 or example 140, wherein the sealing skirt includes a proximal portion and a distal portion, and the pocket is positioned at the proximal portion.

Example 142: The prosthetic valve of any example herein, in particular example 141, further comprising one or more distal anchors, and wherein the distal portion of the sealing skirt is coupled to the one or more distal anchors.

Example 143: The prosthetic valve of any example herein, in particular example 142, wherein each of the one or more distal anchors is configured to hook over a distal tip of a native valve leaflet.

Example 144: The prosthetic valve of any example herein, in particular example 142 or example 143, wherein the distal portion of the sealing skirt is configured to slide along the one or more distal anchors.

Example 145: The prosthetic valve of any example herein, in particular examples 139-144, wherein the ring comprises a proximal tensioning body configured to tension the sealing skirt.

Example 146: The prosthetic valve of any example herein, in particular examples 139-145, wherein the sealing skirt is in tension and configured to deflect to conform to a shape of the native valve.

Example 147: The prosthetic valve of any example herein, in particular examples 139-146, wherein the prosthetic valve comprises a single frame prosthetic valve.

Example 148: The prosthetic valve of any example herein, in particular examples 139-147, wherein the prosthetic valve is configured to be deployed to a mitral valve or a tricuspid valve.

Example 149: A method comprising: deploying a prosthetic valve to a native valve, the prosthetic valve including: a valve frame surrounding a flow channel, one or more prosthetic valve leaflets coupled to the valve frame and extending radially inward from the valve frame and positioned within the flow channel, a sealing skirt positioned radially outward from the valve frame and including a pocket, and a ring positioned in the pocket and configured to support the sealing skirt.

Example 150: The method of any example herein, in particular example 149, wherein the pocket comprises overlapping material of the sealing skirt.

Example 151: The method of any example herein, in particular example 149 or example 150, wherein the sealing skirt includes a proximal portion and a distal portion, and the pocket is positioned at the proximal portion.

Example 152: The method of any example herein, in particular example 151, wherein the distal portion of the sealing skirt is coupled to one or more distal anchors.

Example 153: The method of any example herein, in particular example 152, wherein each of the one or more distal anchors is configured to hook over a distal tip of a native valve leaflet.

Example 154: The method of any example herein, in particular example 152 or example 153, wherein the distal portion of the sealing skirt is configured to slide along the one or more distal anchors.

Example 155: The method of any example herein, in particular examples 149-154, wherein the ring comprises a proximal tensioning body configured to tension the sealing skirt.

Example 156: The method of any example herein, in particular examples 149-155, wherein the sealing skirt is in tension and configured to deflect to conform to a shape of the native valve.

Example 157: A prosthetic valve configured to be deployed to a native valve, the prosthetic valve comprising: a valve frame surrounding a flow channel; one or more prosthetic valve leaflets coupled to the valve frame and extending radially inward from the valve frame and positioned within the flow channel; one or more anchors configured to anchor the one or more prosthetic valve leaflets to the native valve; a sealing skirt positioned radially outward from the valve frame and including a distal portion; and one or more slidable couplers coupling the distal portion of the sealing skirt to the one or more anchors and configured to allow the distal portion of the sealing skirt to slide relative to the one or more anchors.

Example 158: The prosthetic valve of any example herein, in particular example 157, wherein each of the one or more slidable couplers comprises a ring.

Example 159: The prosthetic valve of any example herein, in particular example 158, wherein the ring surrounds a respective one of the one or more anchors and is configured to slide along the one or more anchors.

Example 160: The prosthetic valve of any example herein, in particular examples 157-159, wherein each of the one or more anchors comprises an elongate arm, and the one or more slidable couplers are configured to slide along the elongate arm to allow the distal portion of the sealing skirt to slide relative to the one or more anchors.

Example 161: The prosthetic valve of any example herein, in particular example 160, wherein the elongate arm includes a tip, and the one or more slidable couplers are configured to slide away from the tip.

Example 162: The prosthetic valve of any example herein, in particular example 161, wherein the elongate arm includes a protruding portion positioned between the one or more slidable couplers and the tip, the protruding portion configured to overlap a distal tip of a native valve leaflet.

Example 163: The prosthetic valve of any example herein, in particular example 162, wherein the one or more slidable couplers are configured to slide away from the tip to increase a size of the protruding portion.

Example 164: The prosthetic valve of any example herein, in particular examples 157-163, wherein the sealing skirt is in tension and configured to deflect to conform to a shape of the native valve.

Example 165: The prosthetic valve of any example herein, in particular examples 157-164, wherein the prosthetic valve comprises a single frame prosthetic valve.

Example 166: The prosthetic valve of any example herein, in particular examples 157-165, wherein the prosthetic valve is configured to be deployed to a mitral valve or a tricuspid valve.

Example 167: A method comprising: deploying a prosthetic valve to a native valve, the prosthetic valve including: a valve frame surrounding a flow channel, one or more prosthetic valve leaflets coupled to the valve frame and extending radially inward from the valve frame and positioned within the flow channel, one or more anchors configured to anchor the one or more prosthetic valve leaflets to the native valve, a sealing skirt positioned radially outward from the valve frame and including a distal portion, and one or more slidable couplers coupling the distal portion of the sealing skirt to the one or more anchors and configured to allow the distal portion of the sealing skirt to slide relative to the one or more anchors.

Example 168: The method of any example herein, in particular example 167, wherein each of the one or more slidable couplers comprises a ring.

Example 169: The method of any example herein, in particular example 168, wherein the ring surrounds a respective one of the one or more anchors and is configured to slide along the one or more anchors.

Example 170: The method of any example herein, in particular examples 167-169, wherein each of the one or more anchors comprises an elongate arm, and the one or more slidable couplers are configured to slide along the elongate arm to allow the distal portion of the sealing skirt to slide relative to the one or more anchors.

Example 171: The method of any example herein, in particular example 170, wherein the elongate arm includes a tip, and the one or more slidable couplers are configured to slide away from the tip.

Example 172: The method of any example herein, in particular example 171, wherein the elongate arm includes a protruding portion positioned between the one or more slidable couplers and the tip, the protruding portion configured to overlap a distal tip of a native valve leaflet.

Example 173: The method of any example herein, in particular example 172, wherein the one or more slidable couplers are configured to slide away from the tip to increase a size of the protruding portion.

Example 174: The method of any example herein, in particular examples 167-173, wherein the sealing skirt is in tension and configured to deflect to conform to a shape of the native valve.

Example 175: A prosthetic valve configured to be deployed to a native valve, the prosthetic valve comprising: one or more prosthetic valve leaflets; and one or more anchors configured to anchor the one or more prosthetic valve leaflets to the native valve, each of the one or more anchors comprising a T-bar.

Example 176: The prosthetic valve of any example herein, in particular example 175, wherein each of the one or more anchors comprises an atrial anchor.

Example 177: The prosthetic valve of any example herein, in particular example 175 or example 176, wherein a valve frame supports the one or more prosthetic valve leaflets and has a proximal portion and a distal portion, each of the one or more anchors extending radially outward from the proximal portion of the valve frame.

Example 178: The prosthetic valve of any example herein, in particular example 177, wherein each of the T-bars includes a stem coupled to the proximal portion of the valve frame and a cross bar extending transverse to the stem.

Example 179: The prosthetic valve of any example herein, in particular example 178, wherein each of the T-bars is configured such that the cross bar is positioned radially outward of the stem.

Example 180: The prosthetic valve of any example herein, in particular example 178 or example 179, wherein the one or more anchors comprise a plurality of the anchors, and the cross bars of the plurality of the anchors form a ring extending circumferentially about the prosthetic valve.

Example 181: The prosthetic valve of any example herein, in particular examples 178-180, wherein the one or more anchors comprise a plurality of the anchors, and the cross bars of the plurality of the anchors form a segmented flange extending circumferentially about the prosthetic valve.

Example 182: The prosthetic valve of any example herein, in particular examples 178-181, wherein the stem includes an undulation configured to provide flexibility for the stem.

Example 183: The prosthetic valve of any example herein, in particular examples 178-182, wherein the cross bar includes a central portion coupled to the stem and a first arm and a second arm each extending outward from the central portion, the first arm and the second arm each configured to be deflected radially inward towards the stem.

Example 184: The prosthetic valve of any example herein, in particular examples 175-183, wherein each of the one or more anchors comprises a proximal anchor, and the prosthetic valve further comprises one or more distal anchors each configured to hook over a distal tip of a native valve leaflet.

Example 185: The prosthetic valve of any example herein, in particular example 184, wherein the one or more distal anchors are positioned circumferentially between two of the proximal anchors.

Example 186: The prosthetic valve of any example herein, in particular examples 175-185, further comprising a sealing skirt having a proximal portion and a distal portion, wherein the one or more anchors comprise a proximal tensioning body coupled to the proximal portion of the sealing skirt and configured to tension the sealing skirt.

Example 187: The prosthetic valve of any example herein, in particular example 186, wherein each of the one or more anchors comprises a proximal anchor, and the prosthetic valve further comprises one or more distal anchors each comprising a distal tensioning body coupled to the distal portion of the sealing skirt and configured to tension the sealing skirt.

Example 188: The prosthetic valve of any example herein, in particular examples 175-187, wherein the prosthetic valve comprises a single frame prosthetic valve.

Example 189: The prosthetic valve of any example herein, in particular examples 175-188, wherein the prosthetic valve is configured to be deployed to a mitral valve or a tricuspid valve.

Example 190: A method comprising: deploying a prosthetic valve to a native valve, the prosthetic valve including: one or more prosthetic valve leaflets, and one or more anchors configured to anchor the one or more prosthetic valve leaflets to the native valve, each of the one or more anchors comprising a T-bar.

Example 191: The method of any example herein, in particular example 190, wherein each of the one or more anchors comprises an atrial anchor.

Example 192: The method of any example herein, in particular example 190 or example 191, wherein a valve frame supports the one or more prosthetic valve leaflets and has a proximal portion and a distal portion, each of the one or more anchors extending radially outward from the proximal portion of the valve frame.

Example 193: The method of any example herein, in particular example 192, wherein each of the T-bars includes a stem coupled to the proximal portion of the valve frame and a cross bar extending transverse to the stem.

Example 194: The method of any example herein, in particular example 193, wherein each of the T-bars is configured such that the cross bar is positioned radially outward of the stem.

Example 195: The method of any example herein, in particular example 193 or example 194, wherein the one or more anchors comprise a plurality of the anchors, and the cross bars of the plurality of the anchors form a ring extending circumferentially about the prosthetic valve.

Example 196: The method of any example herein, in particular examples 193-195, wherein the one or more anchors comprise a plurality of the anchors, and the cross bars of the plurality of the anchors form a segmented flange extending circumferentially about the prosthetic valve.

Example 197: The method of any example herein, in particular examples 193-196, wherein the stem includes an undulation configured to provide flexibility for the stem.

Example 198: A prosthetic valve configured to be deployed to a native valve, the prosthetic valve comprising: one or more prosthetic valve leaflets; a valve body supporting the one or more prosthetic valve leaflets; a plurality of distal anchors each comprising an elongate arm having a first end coupled to the valve body and extending radially outward along a length from the first end to a tip of the respective elongate arm, each elongate arm configured to hook over a distal tip of a native valve leaflet to anchor the one or more prosthetic valve leaflets to the native valve; and a sealing skirt forming a disk extending along the length of each of the elongate arms and circumferentially between adjacent of the elongate arms.

Example 199: The prosthetic valve of any example herein, in particular example 198, wherein the sealing skirt is coextensive with each of the elongate arms.

Example 200: The prosthetic valve of any example herein, in particular example 198 or example 199, wherein the sealing skirt extends from the first end to the tip of each of the elongate arms.

Example 201: The prosthetic valve of any example herein, in particular examples 198-200, wherein the valve body includes a frame supporting the one or more prosthetic valve leaflets.

Example 202: The prosthetic valve of any example herein, in particular example 201, wherein the sealing skirt is coextensive with the frame.

Example 203: The prosthetic valve of any example herein, in particular examples 198-202, wherein the disk has a convex curvature.

Example 204: The prosthetic valve of any example herein, in particular examples 198-203, wherein each of the elongate arms includes a curved portion and the disk is configured to extend along the curved portion.

Example 205: The prosthetic valve of any example herein, in particular examples 198-204, wherein at least a portion of the disk is configured to be positioned radially outward of the native valve leaflet.

Example 206: The prosthetic valve of any example herein, in particular examples 198-205, wherein the prosthetic valve comprises a single frame prosthetic valve.

Example 207: The prosthetic valve of any example herein, in particular examples 198-206, wherein the prosthetic valve is configured to be deployed to a mitral valve or a tricuspid valve.

Example 208: A method comprising: deploying a prosthetic valve to a native valve, the prosthetic valve including: one or more prosthetic valve leaflets, a valve body supporting the one or more prosthetic valve leaflets, a plurality of distal anchors each comprising an elongate arm having a first end coupled to the valve body and extending radially outward along a length from the first end to a tip of the respective elongate arm, each elongate arm configured to hook over a distal tip of a native valve leaflet to anchor the one or more prosthetic valve leaflets to the native valve, and a sealing skirt forming a disk extending along the length of each of the elongate arms and circumferentially between adjacent of the elongate arms.

Example 209: The method of any example herein, in particular example 208, wherein the sealing skirt is coextensive with each of the elongate arms.

Example 210: The method of any example herein, in particular example 208 or example 209, wherein the sealing skirt extends from the first end to the tip of each of the elongate arms.

Example 211: The method of any example herein, in particular examples 208-210, wherein the valve body includes a frame supporting the one or more prosthetic valve leaflets.

Example 212: The method of any example herein, in particular example 211, wherein the sealing skirt is coextensive with the frame.

Example 213: The method of any example herein, in particular examples 208-212, wherein the disk has a convex curvature.

Example 214: The method of any example herein, in particular examples 208-213, wherein each of the elongate arms includes a curved portion and the disk is configured to extend along the curved portion.

Example 215: The method of any example herein, in particular examples 208-214, wherein at least a portion of the disk is configured to be positioned radially outward of the native valve leaflet.

Example 216: A prosthetic valve configured to be deployed to a native valve, the prosthetic valve comprising: one or more prosthetic valve leaflets; and one or more anchors configured to anchor the one or more prosthetic valve leaflets to the native valve, each of the one or more anchors including a hinge configured to allow the respective anchor to deflect radially.

Example 217: The prosthetic valve of any example herein, in particular example 216, wherein the hinge is configured to allow the respective anchor to deflect radially inward.

Example 218: The prosthetic valve of any example herein, in particular example 216 or example 217, wherein the hinge comprises a mechanical bearing hinge or a living hinge.

Example 219: The prosthetic valve of any example herein, in particular examples 216-218, wherein the hinge is configured to impede radially outward deflection of the respective anchor.

Example 220: The prosthetic valve of any example herein, in particular examples 216-219, wherein each of the one or more anchors is configured to be in a hooked configuration.

Example 221: The prosthetic valve of any example herein, in particular examples 216-220, wherein each of the one or more anchors includes a first portion and a second portion coupled to the first portion, the first portion extending distally and having a convex curvature in a distal direction, and the second portion extending linearly in a proximal direction to a tip of the respective anchor.

Example 222: The prosthetic valve of any example herein, in particular example 221, wherein the hinge is positioned on the second portion or at a juncture of the first portion to the second portion.

Example 223: The prosthetic valve of any example herein, in particular example 221, wherein the hinge is positioned on the first portion.

Example 224: The prosthetic valve of any example herein, in particular examples 216-223, wherein each of the one or more anchors extends radially outward from a valve body supporting the one or more prosthetic valve leaflets.

Example 225: The prosthetic valve of any example herein, in particular example 224, wherein the valve body includes a proximal end portion and a distal end portion, and each of the one or more anchors is coupled to the distal end portion of the valve body.

Example 226: The prosthetic valve of any example herein, in particular example 224 or example 225, wherein the valve body includes an inner valve body and a sealing body positioned radially outward of the inner valve body.

Example 227: The prosthetic valve of any example herein, in particular example 226, wherein the inner valve body includes an inner frame and the sealing body includes an outer frame.

Example 228: The prosthetic valve of any example herein, in particular example 226 or example 227, wherein the sealing body includes an outer frame and a sealing skirt positioned upon the outer frame.

Example 229: The prosthetic valve of any example herein, in particular examples 226-228, wherein the one or more anchors each comprise an elongate arm.

Example 230: The prosthetic valve of any example herein, in particular examples 226-229, wherein the inner valve body includes a distal end portion and a proximal end portion, and the sealing body includes a distal end portion and a proximal end portion, and the proximal end portion of the inner valve body is coupled to the proximal end portion of the sealing body.

Example 231: The prosthetic valve of any example herein, in particular example 230, wherein the distal end portion of the inner valve body is spaced from the distal end portion of the sealing body with a gap.

Example 232: The prosthetic valve of any example herein, in particular examples 216-231, wherein the hinge is configured to allow the respective anchor to deflect radially inward in response to a radially inward force of a native valve leaflet.

Example 233: The prosthetic valve of any example herein, in particular examples 216-232, wherein the hinge is configured to allow the respective anchor to deflect radially inward in response to a systolic movement of a native valve leaflet.

Example 234: The prosthetic valve of any example herein, in particular examples 216-233, wherein each of the one or more anchors is configured to extend over a distal tip of a native valve leaflet to anchor to the respective native valve leaflet.

Example 235: The prosthetic valve of any example herein, in particular examples 216-234, wherein the prosthetic valve is configured to be deployed to a mitral valve or a tricuspid valve.

Example 236: A method comprising: deploying a prosthetic valve to a native valve, the prosthetic valve including: one or more prosthetic valve leaflets, and one or more anchors configured to anchor the one or more prosthetic valve leaflets to the native valve, each of the one or more anchors including a hinge configured to allow the respective anchor to deflect radially.

Example 237: The method of any example herein, in particular example 236, wherein the hinge is configured to allow the respective anchor to deflect radially inward.

Example 238: The method of any example herein, in particular example 236 or example 237, wherein the hinge comprises a mechanical bearing hinge or a living hinge.

Example 239: The method of any example herein, in particular examples 236-238, wherein the hinge is configured to impede radially outward deflection of the respective anchor.

Example 240: The method of any example herein, in particular examples 236-239, wherein each of the one or more anchors is configured to be in a hooked configuration.

Example 241: The method of any example herein, in particular examples 236-240, wherein each of the one or more anchors includes a first portion and a second portion coupled to the first portion, the first portion extending distally and having a convex curvature in a distal direction, and the second portion extending linearly in a proximal direction to a tip of the respective anchor.

Example 242: The method of any example herein, in particular example 241, wherein the hinge is positioned on the second portion or at a juncture of the first portion to the second portion.

Example 243: The method of any example herein, in particular example 241, wherein the hinge is positioned on the first portion.

Example 244: A prosthetic valve configured to be deployed to a native valve, the prosthetic valve comprising: one or more prosthetic valve leaflets; an inner frame supporting the one or more prosthetic valve leaflets; an outer frame positioned radially outward from the inner frame and surrounding the inner frame, the outer frame being separated from the inner frame with a gap; and one or more anchors coupled to the outer frame and each configured to be positioned radially outward of one or more leaflets of the native valve to anchor to the native valve.

Example 245: The prosthetic valve of any example herein, in particular example 244, wherein each of the one or more anchors is configured to be in a hooked configuration.

Example 246: The prosthetic valve of any example herein, in particular example 244 or example 245, wherein each of the one or more anchors includes a coupling portion and a tip portion, the coupling portion being coupled to the outer frame and the tip portion configured to extend proximally from the coupling portion.

Example 247: The prosthetic valve of any example herein, in particular example 246, wherein the coupling portion has a convex curvature in a distal direction.

Example 248: The prosthetic valve of any example herein, in particular example 246 or example 247, wherein the tip portion is linear.

Example 249: The prosthetic valve of any example herein, in particular examples 246-248, wherein the outer frame includes a distal end portion, and the coupling portion couples to the distal end portion of the outer frame.

Example 250: The prosthetic valve of any example herein, in particular examples 246-249, wherein the outer frame includes a plurality of struts forming a plurality of distal apices, and the coupling portion couples to a respective one of the distal apices.

Example 251: The prosthetic valve of any example herein, in particular examples 246-250, wherein the outer frame includes a plurality of struts forming a lattice structure having a plurality of openings, and the tip portion is configured to be aligned with a respective one of the plurality of openings.

Example 252: The prosthetic valve of any example herein, in particular examples 244-251, wherein the inner frame includes a proximal end portion and a distal end portion, and the outer frame includes a proximal end portion and a distal end portion, the proximal end portion of the inner frame being coupled to the proximal end portion of the outer frame.

Example 253: The prosthetic valve of any example herein, in particular example 252, wherein the distal end portion of the inner frame is separated from the distal end portion of the outer frame with the gap.

Example 254: The prosthetic valve of any example herein, in particular examples 244-253, wherein the one or more anchors are configured to clamp the leaflet of the native valve between the one or more anchors and a portion of the prosthetic valve.

Example 255: The prosthetic valve of any example herein, in particular example 254, wherein the one or more anchors are configured to clamp the leaflet of the native valve between the one or more anchors and the outer frame.

Example 256: The prosthetic valve of any example herein, in particular examples 244-255, wherein the inner frame has a radial thickness, and the outer frame has a radial thickness that is less than the radial thickness of the inner frame.

Example 257: The prosthetic valve of any example herein, in particular examples 244-256, wherein the inner frame has a radial thickness and each of the one or more anchors has a radial thickness that is less than the radial thickness of the inner frame.

Example 258: The prosthetic valve of any example herein, in particular examples 244-257, further comprising a sealing skirt positioned on the outer frame.

Example 259: The prosthetic valve of any example herein, in particular examples 244-258, wherein each of the one or more anchors is configured to be actuated to vary a radial position of the respective anchor.

Example 260: The prosthetic valve of any example herein, in particular example 259, wherein each of the one or more anchors includes a tip portion configured to extend proximally when the prosthetic valve is in a crimped configuration, and each of the one or more anchors is configured to be actuated to deflect the tip portion radially outward from the outer frame.

Example 261: The prosthetic valve of any example herein, in particular example 259 or example 260, wherein the outer frame includes a distal end portion having one or more actuator couplers for coupling with an actuator assembly, the one or more actuator couplers configured to be pulled radially inward by the actuator assembly to deflect the one or more anchors radially outward.

Example 262: The prosthetic valve of any example herein, in particular example 261, wherein the one or more actuator couplers comprise one or more eyelets configured to couple with one or more tethers of the actuator assembly.

Example 263: The prosthetic valve of any example herein, in particular examples 244-262, wherein the prosthetic valve is configured to be deployed to a mitral valve or a tricuspid valve.

Example 264: A method comprising: deploying a prosthetic valve to a native valve, the prosthetic valve including: one or more prosthetic valve leaflets, an inner frame supporting the one or more prosthetic valve leaflets, an outer frame positioned radially outward from the inner frame and surrounding the inner frame, the outer frame being separated from the inner frame with a gap, and one or more anchors coupled to the outer frame and each configured to be positioned radially outward of one or more leaflets of the native valve to anchor to the native valve.

Example 265: The method of any example herein, in particular example 264, wherein each of the one or more anchors is configured to be in a hooked configuration.

Example 266: The method of any example herein, in particular example 264 or example 265, wherein each of the one or more anchors includes a coupling portion and a tip portion, the coupling portion being coupled to the outer frame and the tip portion configured to extend proximally from the coupling portion.

Example 267: The method of any example herein, in particular example 266, wherein the coupling portion has a convex curvature in a distal direction.

Example 268: The method of any example herein, in particular example 266 or example 267, wherein the tip portion is linear.

Example 269: The method of any example herein, in particular examples 266-268, wherein the outer frame includes a distal end portion, and the coupling portion couples to the distal end portion of the outer frame.

Example 270: The method of any example herein, in particular examples 266-269, wherein the outer frame includes a plurality of struts forming a plurality of distal apices, and the coupling portion couples to a respective one of the distal apices.

Example 271: The method of any example herein, in particular examples 266-270, wherein the outer frame includes a plurality of struts forming a lattice structure having a plurality of openings, and the tip portion is configured to be aligned with a respective one of the plurality of openings.

Example 272: A prosthetic valve configured to be deployed to a native valve, the prosthetic valve comprising: one or more prosthetic valve leaflets; and a frame including an outer frame and an inner frame, the outer frame and the inner frame being cut from a single piece of material such that the outer frame is integral with the inner frame, the outer frame being deflected radially outward from the inner frame and including a plurality of struts bounding a plurality of openings, the inner frame configured to support the one or more prosthetic valve leaflets and including a plurality of struts bounding a plurality of openings, and at least a portion of the inner frame is aligned within at least one of the plurality of openings of the outer frame, or at least a portion of the outer frame is aligned within at least one of the plurality of openings of the inner frame.

Example 273: The prosthetic valve of any example herein, in particular example 272, wherein the frame is configured to expand radially outward from a crimped configuration to an expanded configuration, the inner frame and the outer frame each having a cylindrical shape in the crimped configuration, and the outer frame having an hourglass shape in the expanded configuration.

Example 274: The prosthetic valve of any example herein, in particular example 273, wherein the inner frame has a cylindrical shape in the expanded configuration.

Example 275: The prosthetic valve of any example herein, in particular example 273 or example 274, wherein at least a portion of the inner frame is configured to be positioned within at least one of the plurality of openings of the outer frame when the frame is in the crimped configuration.

Example 276: The prosthetic valve of any example herein, in particular examples 273-275, wherein at least a portion of the outer frame is configured to be positioned within at least one of the plurality of openings of the inner frame when the frame is in the crimped configuration.

Example 277: The prosthetic valve of any example herein, in particular examples 272-276, wherein the plurality of struts of the outer frame forms a plurality of closed strut cells, at least a portion of the inner frame being aligned within at least one of the plurality of closed strut cells.

Example 278: The prosthetic valve of any example herein, in particular examples 272-277, wherein the plurality of struts of the outer frame forms a plurality of open strut cells, at least a portion of the inner frame being aligned within at least one of the plurality of open strut cells.

Example 279: The prosthetic valve of any example herein, in particular examples 272-278, wherein the plurality of struts of the outer frame forms a plurality of strut cells each having a diamond shape.

Example 280: The prosthetic valve of any example herein, in particular examples 272-279, wherein the plurality of struts of the inner frame forms a plurality of strut cells each having a diamond shape.

Example 281: The prosthetic valve of any example herein, in particular examples 272-280, wherein the plurality of struts of the outer frame forms a diamond shaped strut cell, and the plurality of struts of the inner frame form a diamond shaped strut cell aligned within the diamond shaped strut cell of the outer frame.

Example 282: The prosthetic valve of any example herein, in particular examples 272-281, wherein the plurality of struts of the inner frame forms a diamond shaped strut cell, and the plurality of struts of the outer frame form a diamond shaped strut cell aligned within the diamond shaped strut cell of the inner frame.

Example 283: The prosthetic valve of any example herein, in particular examples 272-282, wherein the plurality of struts of the inner frame form at least three diamond shaped strut cells each circumferentially spaced from each other.

Example 284: The prosthetic valve of any example herein, in particular examples 272-283, wherein the outer frame includes a proximal portion, a distal portion, and a central portion, and the proximal portion is configured to flare radially outward from the central portion, and the distal portion is configured to flare radially outward from the central portion.

Example 285: The prosthetic valve of any example herein, in particular example 284, wherein the outer frame is configured to clamp a portion of a heart between the proximal portion and the distal portion to anchor the frame to the heart.

Example 286: The prosthetic valve of any example herein, in particular examples 272-285, wherein the outer frame is configured to anchor the frame to the native valve.

Example 287: The prosthetic valve of any example herein, in particular examples 272-286, further comprising one or more anchors coupled to the outer frame and configured to form hooks for anchoring to the native valve.

Example 288: The prosthetic valve of any example herein, in particular examples 272-287, further comprising one or more anchors coupled to the inner frame and configured to form hooks for anchoring to the native valve.

Example 289: The prosthetic valve of any example herein, in particular examples 272-288, further comprising one or more anchors coupled to the outer frame or the inner frame and configured to form hooks for clamping a leaflet of the native valve.

Example 290: The prosthetic valve of any example herein, in particular examples 272-289, further comprising a sealing skirt positioned upon the outer frame.

Example 291: The prosthetic valve of any example herein, in particular examples 272-290, wherein the prosthetic valve is configured to be deployed to a mitral valve or a tricuspid valve.

Example 292: A method comprising: deploying a prosthetic valve to a native valve, the prosthetic valve including: one or more prosthetic valve leaflets, a frame including an outer frame and an inner frame, the outer frame and the inner frame being cut from a single piece of material such that the outer frame is integral with the inner frame, the outer frame being deflected radially outward from the inner frame and including a plurality of struts bounding a plurality of openings, the inner frame configured to support the one or more prosthetic valve leaflets and including a plurality of struts bounding a plurality of openings, and at least a portion of the inner frame is aligned within at least one of the plurality of openings of the outer frame, or at least a portion of the outer frame is aligned within at least one of the plurality of openings of the inner frame.

Example 293: The method of any example herein, in particular example 292, wherein the frame is configured to expand radially outward from a crimped configuration to an expanded configuration, the inner frame and the outer frame each having a cylindrical shape in the crimped configuration, and the outer frame having an hourglass shape in the expanded configuration.

Example 294: The method of any example herein, in particular example 293, wherein the inner frame has a cylindrical shape in the expanded configuration.

Example 295: The method of any example herein, in particular example 293 or example 294, wherein at least a portion of the inner frame is configured to be positioned within at least one of the plurality of openings of the outer frame when the frame is in the crimped configuration.

Example 296: The method of any example herein, in particular examples 293-295, wherein at least a portion of the outer frame is configured to be positioned within at least one of the plurality of openings of the inner frame when the frame is in the crimped configuration.

Example 297: The method of any example herein, in particular examples 292-296, wherein the plurality of struts of the outer frame forms a plurality of closed strut cells, at least a portion of the inner frame being aligned within at least one of the plurality of closed strut cells.

Example 298: The method of any example herein, in particular examples 292-297, wherein the plurality of struts of the outer frame forms a plurality of open strut cells, at least a portion of the inner frame being aligned within at least one of the plurality of open strut cells.

Example 299: The method of any example herein, in particular examples 292-298, wherein the plurality of struts of the outer frame forms a plurality of strut cells each having a diamond shape.

Example 300: The method of any example herein, in particular examples 292-299, wherein the prosthetic valve is configured to be deployed to a mitral valve or a tricuspid valve.

Any of the features of any of the examples, including but not limited to any of the first through 300 examples referred to above, is applicable to all other aspects and examples identified herein, including but not limited to any examples of any of the first through 300 examples referred to above. Moreover, any of the features of an example of the various examples, including but not limited to any examples of any of the first through 300 examples referred to above, is independently combinable, partly or wholly with other examples described herein in any way, e.g., one, two, or three or more examples may be combinable in whole or in part. Further, any of the features of the various examples, including but not limited to any examples of any of the first through 300 examples referred to above, may be made optional to other examples. Any example of a method can be performed by a system or apparatus of another example, and any aspect or example of a system or apparatus can be configured to perform a method of another aspect or example, including but not limited to any examples of any of the first through 300 examples referred to above.

In closing, it is to be understood that although aspects of the present specification are highlighted by referring to specific examples, one skilled in the art will readily appreciate that these disclosed examples are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular examples only and is not intended to limit the scope of systems, apparatuses, and methods as disclosed herein, which is defined solely by the claims. Accordingly, the systems, apparatuses, and methods are not limited to that precisely as shown and described.

Certain examples of systems, apparatuses, and methods are described herein, including the best mode known to the inventors for carrying out the same. Of course, variations on these described examples will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described examples in all possible variations thereof is encompassed by the systems, apparatuses, and methods unless otherwise indicated herein or otherwise clearly contradicted by context.

Groupings of alternative examples, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A prosthetic valve for deployment to a native valve, the prosthetic valve comprising:
a valve frame surrounding a flow channel;
one or more prosthetic valve leaflets coupled to the valve frame and extending radially inward from the valve frame and positioned within the flow channel;
a proximal tensioning body positioned at a proximal portion of the prosthetic valve;
a distal tensioning body positioned at a distal portion of the prosthetic valve; and
a sealing skirt including a proximal end portion and a distal end portion and positioned radially outward from the valve frame with a space between the sealing skirt and the valve frame, the proximal end portion of the sealing skirt coupled to the proximal tensioning body and the distal end portion of the sealing skirt coupled to the distal tensioning body, the sealing skirt being tensioned by the proximal tensioning body and the distal tensioning body and being deflectable to conform to a shape of the native valve, and
wherein at least one of the proximal tensioning body or the distal tensioning body comprises a ring extending circumferentially about the prosthetic valve.

2. The prosthetic valve of claim 1, wherein the sealing skirt surrounds the valve frame, and a central portion of the sealing skirt is uncoupled from the valve frame and is free from the valve frame.

3. The prosthetic valve of claim 1, wherein the sealing skirt is elastic and configured to deflect radially inward to conform to the shape of the native valve.

4. The prosthetic valve of claim 1, wherein the proximal tensioning body and the distal tensioning body each comprise a ring extending circumferentially about the prosthetic valve.

5. The prosthetic valve of claim 1, wherein the ring is compressible and biased to expand radially outward.

6. The prosthetic valve of claim 1, wherein the ring includes overlapping end portions.

7. The prosthetic valve of claim 1, wherein the distal tensioning body comprises a distal anchor for the prosthetic valve.

8. The prosthetic valve of claim 7, wherein the distal anchor has a hook shape.

9. The prosthetic valve of claim 8, further comprising a slidable coupler coupled to the distal end portion of the sealing skirt and coupled to the distal anchor, the slidable coupler adapted to slide relative to the distal anchor.

10. The prosthetic valve of claim 1, wherein the sealing skirt includes a pocket, and the ring is positioned within the pocket of the sealing skirt.

11. The prosthetic valve of claim 1, wherein the prosthetic valve is a mitral prosthetic valve or a tricuspid prosthetic valve.

12. A system for treatment of a native heart valve, the system comprising:
a prosthetic valve for deployment to the native heart valve, the prosthetic valve including:
a valve frame surrounding a flow channel,
one or more prosthetic valve leaflets coupled to the valve frame and extending radially inward from the valve frame and positioned within the flow channel,
a proximal tensioning body positioned at a proximal portion of the prosthetic valve,
a distal tensioning body positioned at a distal portion of the prosthetic valve, and
a sealing skirt including a proximal end portion and a distal end portion and positioned radially outward from the valve frame with a space between the sealing skirt and the valve frame, the proximal end portion of the sealing skirt coupled to the proximal tensioning body and the distal end portion of the sealing skirt coupled to the distal tensioning body, the sealing skirt being tensioned by the proximal tensioning body and the distal tensioning body and being deflectable to conform to a shape of the native heart valve, and
wherein at least one of the proximal tensioning body or the distal tensioning body comprises a ring extending circumferentially about the prosthetic valve; and
a delivery apparatus including an elongate shaft for being advanced through vasculature of a patient to the native heart valve, the delivery apparatus including an implant retention area for retaining the prosthetic valve.

13. The system of claim 12, wherein the sealing skirt surrounds the valve frame, and a central portion of the sealing skirt is uncoupled from the valve frame and is free from the valve frame.

14. The system of claim 12, wherein the proximal tensioning body and the distal tensioning body each comprise a ring extending circumferentially about the prosthetic valve.

15. A prosthetic valve configured to be deployed to a native valve, the prosthetic valve comprising:
one or more prosthetic valve leaflets;
an inner frame supporting the one or more prosthetic valve leaflets;
an outer frame positioned radially outward from the inner frame and surrounding the inner frame, the outer frame having a proximal end portion and a distal end portion and being separated from the inner frame with a gap; and
one or more anchors coupled to the distal end portion of the outer frame and each shaped to be positioned radially outward of one or more leaflets of the native valve to anchor to the native valve.

16. The prosthetic valve of claim 15, wherein each of the one or more anchors comprises a hook.

17. The prosthetic valve of claim 15, wherein each of the one or more anchors includes a coupling portion and a tip portion, the coupling portion being coupled to the outer frame and the tip portion shaped to extend proximally from the coupling portion.

18. The prosthetic valve of claim 17, wherein the coupling portion has a convex curvature in a distal direction.

19. The prosthetic valve of claim 17, wherein the outer frame includes a plurality of struts forming a plurality of distal apices, and the coupling portion couples to a respective one of the distal apices.

20. The prosthetic valve of claim 15, wherein at least one of the one or more anchors includes a hinge adapted to allow the anchor to deflect radially.
